SEQUENCE LISTING (16 sequences)

SEQ ID NO: 1 - DNA, 9181 bp, Human immunodeficiency virus type 1
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatttgggtcagggagtctccatagaatggaggaaaaagagatatagcacacaagtagaccctgaactagcagaccaactaattcatctgtattactttgactgtttttcagactctgctataagaaaggccttattaggacacatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccattttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataagatagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttc

SEQ ID NO: 2 - DNA, 44 bp, Human immunodeficiency virus type 1
ggtctctctggttagaccagatctgagcctgggagctctctggc

SEQ ID NO: 3 - DNA, 117 bp, Human immunodeficiency virus type 1
cttaagcctcaataaagcttgccttgagtgcttcaagtagtgtgtgcccgtctgttgtgtgactctggtaactagagatccctcagacccttttagtcagtgtggaaaatctctagc

SEQ ID NO: 4 - DNA, 113 bp, Human immunodeficiency virus type 1
ctgaagcgcgcacggcaagaggcgaggggcggcgactggtgagtacgccaaaaattttgactagcggaggctagaaggagagagatgggtgcgagagcgtcagtattaagcgg

SEQ ID NO: 5 - DNA, 92 bp, Human immunodeficiency virus type 1
gcagggagctagaacgattcgcagttaatcctggcctgttagaaacatcagaaggctgtagacaaatactgggacagctacaaccatccctt

SEQ ID NO: 6 - DNA, 168 bp, Human immunodeficiency virus type 1
gtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatg

SEQ ID NO: 7 - DNA, 163 bp, Human immunodeficiency virus type 1
aatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggag

SEQ ID NO: 8 - DNA, 59 bp, Human immunodeficiency virus type 1
ggaccaaaggaaccctttagagactatgtagaccggttctataaaactctaagagccga

SEQ ID NO: 9 - DNA, 45 bp, Human immunodeficiency virus type 1
cagcatgtcagggagtaggaggacccggccataaggcaagagttt

SEQ ID NO: 10 - DNA, 58 bp, Human immunodeficiency virus type 1
ttagtggggaaattgaattgggcaagtcagatttacccagggattaaagtaaggcaat

SEQ ID NO: 11 - DNA, 52 bp, Human immunodeficiency virus type 1
gagcaatcacaagtagcaatacagcagctaccaatgctgcttgtgcctggct

SEQ ID NO: 12 - DNA, 122 bp, Human immunodeficiency virus type 1
gtgttagagtggaggtttgacagccgcctagcatttcatcacgtggcccgagagctgcatccggagtacttcaagaactgctgacatcgagcttgctacaagggactttccgctggggactt

SEQ ID NO: 13 - DNA, 53 bp, Human immunodeficiency virus type 1
cctgtactgggtctctctggttagaccagatctgagcctgggagctctctggc

SEQ ID NO: 14 - protein, 39 aa, Human immunodeficiency virus type 1
QLQARILAVERYLKDQQLLGIWGCSGKLICTTAVPWNAS

SEQ ID NO: 15 - protein, 231 aa, Human immunodeficiency virus type 1
PIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVL

SEQ ID NO: 16 - protein, 264 aa, Human immunodeficiency virus type 1
SELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSVVNRVRQGYSPLSFQTHLPIPRGPDRPEGIEEEGGERDRDR

(19) United States
(12) Patent Application Publication
(10) Pub. No.: US 2010/0105024 A1
(43) Pub. Date: Apr. 29, 2010

(54) RAPID TEST INCLUDING GENETIC SEQUENCE PROBE

(75) Inventors: Weidong Xu, Tampa, FL (US); Shyam Mohapatra, Lutz, FL (US); Arun Kumar, Wesley Chapel, FL (US)

Correspondence Address: CHRISTOPHER PARADIES, PH.D., FOWLER WHITE BOGGS P.A., 501 E KENNEDY BLVD, STE. 1700, TAMPA, FL, 33602, US

(73) Assignee: Transgenex Nanobiotech, Inc., Tampa, FL (US)

(21) Appl. No.: 12/424,903

(22) Filed: Apr. 16, 2009

Related U.S. Application Data
(63) Continuation of application No. PCT/US09/31011, filed on Jan. 14, 2009.
Application No. PCT/US09/31011 is a continuation-in-part of application No. 12/008,861, filed on Jan. 14, 2008.
Application No. 12/008,861 is a continuation-in-part of application No. 12/008,861, filed on Jan. 14, 2008.
(60) Provisional application No. 61/118,939, filed on Dec. 1, 2008.

(51) Int. Cl. C12Q 1/70; C12Q 1/68; G01N 33/544

(52) U.S. Cl. 435/5; 435/6; 436/530

(57) ABSTRACT

A rapid test kit may have a genetic probe, and antibody detecting probe or a combination of a genetic probe and an antibody detecting probe disposed within one or more test windows of the test kit. A cellulose filter paper membrane with a flow rate selected in a range of about 0.04 to about 0.4 ml/min/cm² is used in one example. The test kit provides for rapid screening for DNA, RNA or fragments of DNA or RNA in a bodily fluid or antibodies indicating exposure to such DNA/RNA. The genetic probe may include single stranded DNA or a fragment of single stranded DNA, such as primer, immobilized on the filter paper, and a single stranded DNA, such as the same or a different primer, conjugated with a marker, such as a nanotube or nanoparticle. For example, a gold nanoparticle or a carbon nanotube may be used as a staining agent by conjugating the gold nanoparticle or the carbon nanotube to a genetic probe, such as a DNA primer capable of binding with a complementary DNA or viral RNA or a fragment of one of these. By comparing contrast or intensity of a test spot to a standard, a viral load may be reported. By comparing a test region using the genetic marker and a test region using an antigen to detect antibodies, a sensitive and specific test may be conducted during use of a vaccine to determine the effectiveness of the vaccine, for example.

### RELATED APPLICATION

This application is a continuation of International Application PCT/US09/31011 filed on Jan. 14, 2009 which claims priority to U.S. Provisional Application 61/118,939, filed on Dec. 1, 2008, and to U.S. patent application Ser. No. 12/008,861 filed on Jan. 14, 2008, which are both incorporated by reference herein in their entirety. This application is also a continuation-in-part of U.S. patent application Ser. No. 12/008,861 filed on Jan. 14, 2008. The color photographs of U.S. patent application Ser. No. 12/008,861, including FIGS. 2, 3, 4A-B, 6, 11-14, and the description relating to these figures, including paragraphs [0069]-[0070], [0328]-[0331], [0343]-[0344], [0347], [0349]-[0350], [0352]-[0358], and [0361] are incorporated herein by reference for the purposes of disclosing the unexpected advantages of some examples of the invention and a color scale used for quantitative evaluation.

### SEQUENCE LISTING

A compact disk including the sequence listings of SEQ. ID. NOS. 1-16 is incorporated by reference herein. A copy of the sequence listings is available in electronic form from the USPTO upon request.

### FIELD OF THE INVENTION

The field is test kits providing rapid detection and diagnosis of an infectious agent, RNA or DNA in a volume of fluid containing enough antibodies, RNA, DNA or fragments thereof for detection of antibodies or sequences of DNA or RNA by the test kit.

### BACKGROUND

Many diseases are first diagnosed using screening tests and are confirmed by additional testing. It is known that screening tests must possess a high degree of sensitivity, whereas confirmatory assays must possess a high degree of specificity. Tests with high sensitivity are known to produce few false-negative results, whereas tests with high specificity produce few false-positive results. It is difficult to produce a test kit having both high sensitivity and a high degree of specificity. Those knowledgeable in the field recognize that a single kit for use in a field, home environment, or a doctor's office cannot meet both sensitivity and specificity in a rapid assay for diseases diagnosed by testing for viral and bacterial antibodies, such as antibodies for AIDS (e.g., HIV), tuberculosis, malaria, and hepatitis, for example. Instead, field tests are used only for screening and more specific tests are conducted in a controlled laboratory environment.

Blood may be stored for 7-14 days in order to screen for a virus, increasing risks for anaphylactic reactions, increasing potassium concentration, and decreasing its oxygen carrying capacity. There is a longstanding need for agencies to conduct local blood tests to screen donors. The ability to screen bodily fluids, such as blood, saliva and urine, using reliable and rapid test kits is an unfilled and longstanding need.

The most common screening test is the enzyme-linked immunosorbent assay (ELISA), sometimes called enzyme immunoassay (EIA). The most often used confirmatory test is the Western blot. If antibodies are being produced in the body, these tests are capable of detecting the antibodies at low levels. For example, the conventional HIV testing protocol starts with a sensitive EIA in a clinical laboratory. The EIA might be performed with serum, plasma, urine, or oral fluid, and the results might be available in 3 to 4 days. If the EIA is negative, the result is considered definitive, and no further testing is indicated.

A limitation of any testing is that many viral antibodies take up to 3 months to express after infection occurs, causing a window between the infection and detection using even the most sensitive of assays. If the EIA is repeatedly positive, more specific testing, using the Western blot technique, is done for confirmation. The testing process from the time a specimen is submitted until a final result is available is often a week or even longer. The cost and time required to complete a test make frequent testing, even among high risks groups, impractical.

The Western blot test (WB) uses an electrical field that separates out the various components of a sample by molecular weight. This allows identification of antibodies to specific viral antigens, which show up as identifiable “bands” on a strip of test paper. This test offers a high degree of specificity. ELISA combines the specificity of antibodies with the sensitivity of simple enzyme assays, by using antibodies or antigens coupled to an easily assayed enzyme that possesses a high turnover number. ELISA can provide a useful measurement of antigen or antibody concentration, which is unavailable in rapid test kits. Herein, a rapid test is one that provides for a buffered specimen of blood or serum to be used in a test requiring less than five minutes to complete.

Whereas ELISA measures an antibody to a whole virus and gives a “positive,” “negative” or indeterminate test result, Western blotting is a more specific test. It allows one to visualize antibodies directed against each viral protein. For this reason, it is a confirmatory test for a positive test done with ELISA or EIA. The Western blot test is considered a gold standard test for the confirmation of an ELISA and/or a rapid assay screened reactive sample in the diagnosis of many viral infections, especially in the low risk population. Essentially, any repeatedly positive result by ELISA or another rapid screening method for many viral infections must be confirmed by a more specific assay such as a Western blot (WB) test.

This strategy of using both ELISA and supplementary tests further increases the accuracy of results and diagnosis. In principle, any WB kit that gives a high frequency of indeterminate reactivity (the overwhelming preponderance of which represents non-specific binding) is not appropriate as a primary screening tool for the population at large. Its strength is only as a confirmatory assay in the setting of a positive or indeterminate viral antibody ELISA or initial screening test.

In one example, the window period is the period between the onset of viral infection and the appearance of detectable antibodies to the virus. In the case of the most sensitive HIV antibody tests currently recommended, the window period is about three to four weeks. This period can, however, be longer. Any antibody-based blood test (such as the ELISA, rapid tests and the Western blot) conducted during this window period may give false negative results. The expense and time that these tests take means that testing is conducted infrequently on individuals. Although the virus is present in the person's blood there may be no (detectable) antibodies in the blood during a screening test for a period up to about three months, but the cost of testing increases this window to a year or more, especially if the individual is in a low risk group. Indeed, the onset of symptoms of disease is often the first indication in most patients. Waiting until the onset of symptoms of disease has the potential of exposing others to disease and dramatically diminishes the ability to treat a patient, in most cases. This is true for AIDS, hepatitis, tuberculosis and many other diseases that are proving increasingly difficult to treat, at least for some strains, with conventional antiviral or antibiotic regimens. During this window period and until a subsequent test is performed, the individual is already infectious and may unknowingly infect other people. What is needed is a rapid, inexpensive and sensitive test for detecting infectious diseases that permits routine testing of individuals at office visits, testing sites, blood donation centers, or even at home.

There has been an increase in the number of test kits for detecting infectious agents, such as viral and bacterial diseases. Unfortunately, there are many examples of test kits marketed for home use that are neither approved nor adequately tested for diseases such as AIDS. The only approved test kit for HIV in the United States takes a sample and sends the sample to a laboratory for analysis. No known rapid test kits that do not require sending a sample to a laboratory are approved for use in screening for HIV in the United States.

Some test kits are available for testing serum samples for disease. For example, test kits are available that include lateral flow tests. Lateral flow tests, also called immunochromatographic strip tests, are used for specific screening or semi-quantitative detection of many analytes including antigens and antibodies. Samples may either be used alone or with an extraction reagent, or running buffer, which is then placed on a sample pad on one end of a test strip. The test strip also includes a membrane. A signal reagent, is solubilized and binds to an antigen if present in the sample and moves through the membrane by capillary action. The complex is then captured by a second antibody, which produces a visible line, indicating presence of the antigen. Lateral flow tests are slow, but contrast is improved between the visible line and the background compared to directly depositing the sample in the test area. For this reason, lateral flow tests dominate the market for enzymatic testing of bodily fluids. No lateral test is known that is capable of using blood.

Lateral-flow dipstick test kits are known that can detect DNA, as reported in Glynou K (2003), but the shortcomings of lateral-flow disptick tests for blood and other bodily fluids are not solved, and no detection of RNA has been reported using a lateral-flow dipstick test.

Flow through tests may involve kits as individual cassettes with extraction and wash buffers included. These tests involve capturing of an analyte such as antibody or an antigen by a reagent as it flows through a membrane. These test kits often suffer from poor contrast. The protocols may require a user to prepare the sample to be tested, to wash the membrane, to add a signal reagent, and to wash the membrane to clear the membrane of any residue from the sample in an attempt to improve the contrast between the background and any screening line or marker for indicating the presence of an enzyme or antibody. Direct, flow-through test kits are known to be rapid but are seldom used in practice due to the complexity of the protocol required to provide enough contrast between the indicator and the background membrane. Within the field, there was a general acceptance that lack of contrast makes flow through test kits less sensitive than lateral flow test kits, and this taught away from the use of flow through test kits. Also, it was though that complicated procedures and instructions were necessary for washing and rewashing the kits making results, in practice, less consistent than results for a lateral flow test kit, which also mitigates against flow-through tests. Examples of testing with several other commercial tests kits are reported and compared here with examples of the present invention using antigens for detecting HIV antibodies. None of the commercial samples tested with whole blood worked, which limits the usefulness of any of these commercial test kits for field use where a laboratory and centrifuge are not available. Also, some examples had better contrast than commercial test kits, which makes them much easier to read.

Chen in WO 96/21863, describes an immunoassay test device for detection of antibodies to HIV-1 and HIV-2 in biological fluid, providing for immediate immunoreaction and detection of the presence of such antibodies, comprising an assembled filter device and reaction cell using a nitrocellulose membrane on which an immunoreaction occurs. Visualizing the antibodies that react with HIV antigenic glycoproteins gp41, gp36, gp38 and gp120 occurs by conjugating the antibodies with a Protein A colloidal indicator and viewing the membrane for the presence of a red color, indicating the presence of antibodies. Chen teaches a lateral flow and/or filtering of blood through a filtration medium before contacting a nitrocellulose membrane. The extra step of filtration first before contacting the membrane increases the time required for performing the test. Chen, in another publication, WO 95/18624, teaches a similar device that requires a nitrocellulose membrane. In this test, Chen uses only one protein, gp41. Western blot tests require presence of two of three HIV proteins for improved specificity; however, increasing the number of proteins detected does not reliably lead to improved sensitivity and specificity. In some cases, Western blot provides an indeterminate result that may actually indicate a specimen positive for HIV. Abbott Determine™ is an early screening test for HIV 1 and 2, but it does not provide a rapid test kit capable of use in the field with whole blood, for example.

Mahajan, in US Patent Publication No. 2004/0023210, discloses a diagnostic kit for detection of antibodies of Hepatitis C virus in human serum and plasma, which comprises a base, an immunofiltration membrane of nitrocellulose mounted over an absorbent pad disposed on the base, and a top cover removably attached to the base having a central hole conforming to the membrane's circumference. Antigens such as NS3, NS4, and NS5 are immobilized on the membrane and visualized with a Protein A conjugate. This reference teaches that the pore size of the nitrocellulose membrane is 0.8-1.5 microns. The pore size is poorly correlated with specificity and sensitivity, which are correlated with contrast (or color index values as reported herein). Test kits suitable only for use with serum or plasma are not suitable for use as rapid field test kits.

Hu, in U.S. Patent Publication No. 2003/0165970, teaches a diagnostic device for simultaneously detecting multiple infectious agents, such as HIV antibodies, Hepatitis B and C antibodies and syphilis antibody. The kit disclosed by Hu comprises an immunogold filtration assay device, buffer and a mixture of colloidal gold particles where the device includes a nitrocellulose membrane blotted with HBsAg monoclonal antibody, HCV antigen, syphilitic antigen, HIV antigen, and goat anti-mouse IgG antibody. The test is not rapid and requires a very complicated protocol.

Chu, in U.S. Pat. No. 5,885,526, discloses a flow-through test device having a reaction membrane that includes porous material, such as nitrocellulose. A small pore size is taught to be needed when using nitrocellulose membranes in order to provide a greater area for immobilizing receptor molecules. Chu teaches that larger pore sizes lead to decreased assay sensitivity, as described in col. 5, Ins. 53-56. Chu prefers the porosity of the reaction membrane to be in a range from 0.45 to 3 microns. Chu teaches away from using compression to hold the reaction membrane, as it makes the device less suitable for some immunoassays where quantitative results are needed, as disclosed in col. 3, lns. 15-32, and Chu fails to disclose any example using whole blood with cellulose filter papers.

The examples in Chu also teach away from increasing flow rate, which Chu describes as decreasing interaction time between a target molecule in the sample and an immobilized receptor on the reaction membrane. Thus, assay sensitivity decreases as disclosed in col. 5, lns. 57-60. Again, pore size is a poor predictor of sensitivity and specificity.

Chu also teaches that a thick reaction membrane is needed to form an air pocket to prevent lateral flow and direct flow. The working example discloses a thick 800 micron paper-backed nitrocellulose reaction membrane, as disclosed in Example 2. Chu also discloses many disadvantages of prior art devices which have thin reaction membranes such as membranes being less than 0.1 mm thick, as disclosed in col. 7, lns. 66-col. 8.

Chu, discloses that a membrane should be capable of immobilizing an antigen and Protein A and he suggests materials such as nitrocellulose and fiberglass as being suitable for immobilizing the antigen and Protein A. Chu requires an inoculation of both Protein A and an antigen at different areas of the membrane before testing of an analyte sample. Chu also requires both protein A and an antigen of interest to be inoculated on the membrane first before a serum sample is absorbed into the membrane and also utilizes an additional step of adding protein A-colloidal gold conjugate to be added after the serum or plasma is absorbed, which makes Chu's preferred protocol, which is necessary to provide adequate contrast, very complex and not at all rapid. In addition, Chu discloses inoculation of Protein A to be preferably at an edge of a device, as the central location of the membrane will contain an antigen of interest, such as a Hepatitis C antigen. Chu in another patent, U.S. Pat. No. 5,541,059, discloses an immunoassay device employing Protein A and an antigen. The test kits of Chu are not rapid test kits and suffer from complicated protocols, and unpredictable results in the hands of less trained staff and individuals. Chen et al., in U.S. Patent Publication No. 2004/0002063, prefers a porous reaction membrane such as paper-backed nitrocellulose, and a preferred pore size of 0.2 to 0.8 microns, as disclosed in paragraph [0062]. The membranes disclosed in Chen must be suitably porous membranes, such as the examples disclosed that use a nitrocellulose backed with porous paper. Testing of nitrocellulose membranes show that flow rate of water through the membranes are very rapid, but nitrocellulose failed in tests conducted by the applicant. While Chen does not exclude cellulose filter paper as a membrane, cellulose filter paper having a flow rate comparable to nitrocellulose is inoperable, as shown by the applicants results. No examples are provided by Chen using cellulose filter paper as a membrane in any test kit. Also, Gelman et al., in U.S. Pat. No. 5,980,746, teaches away from the use of cellulose compounds because it is well known in the art that cellulose compounds, “reduce membrane adsorbability of proteins,” for example. Thus, it is known to use nitrocellulose membranes in testing for the presence of antibodies.

In addition, for testing blood, Chen discloses that a more complex test kit having a separate blood separation zone is needed, such as one using a glass fiber matrix as the blood separation material, an example provided in paragraph [0089] of U.S. Patent Publication No. 2004/0002063, for example. This complicated procedure is not viable as a field test.

Krutzik, in U.S. Pat. No. 6,653,066, discloses a lateral flow test using a matrix pore size of less than 5 microns and nitrocellulose membranes and discourages the use of larger pore sizes, which tends to have poor results.

It is well-known to collect dried whole-blood spots on cellulose filter paper, but this is used for collection and drying of blood and not as a rapid test kit. Indeed, the characteristics that make cellulose filter papers attractive for storing blood are counterintuitive for test kits. The dried blood is later washed from the filter paper and is used for testing. For example, Rocks et al., in Ann. Clinical Biochemistry 1991; 28:155-159, describes collecting blood on filter paper before evaluating the samples in an antigen coated microtitration wells, using a silver-enhanced gold-labelled immunoassay. Patton et al., in Clinical and Vaccine Immunology, January 2006, pgs. 152-153, describes the use of filter paper for gathering blood samples for further analysis with HIV-1 p24 ELISA assay, but this does not use the cellulose filter paper as a membrane or in the test. Instead, the blood is washed from the filter paper and is used in a separate test. Fortes et al., in Journal of Clinical Microbiology, June 1989, pgs. 1380-1381, describes the use of a cotton filter paper before conducting further ELISA tests. None of these references disclose any type of rapid test kit. Instead, the filter paper is used to transport and store dried blood, which is washed from the filter paper.

One of the problems with using antibodies/antigens for detecting the presence of a disease is that subjects that have received an immunization or that have an immune response may have antibodies to a virus or bacteria but not the disease. For example, a vaccine against AIDS usually comprises a complex mixture of HIV-1 epitopes (peptides, proteins, DNA expression plasmids, and recombinant viral vectors) and can elicit persistent antibody responses in vaccinated volunteers that are detectable by FDA-licensed HIV-1 detection kits. Vaccine-induced antibodies can cause false positives or indeterminate reactivity when sera of vaccinated volunteers are tested using existing serological detection assays. In another example, infants of mothers infected with HIV may test positive for HIV antibodies, because the infant's immune system is influenced by maternal antibodies for an uncertain duration.

Testing for viral genetic material is possible using advanced PCR and reverse transcription PCR techniques, but these techniques are not appropriate for a rapid test kit for use at point of care. Instead, these techniques are expensive and time consuming. Advances in PCR on a chip and the like offer some promise for reducing costs and allowing point of care PCR methods, but practical, commercial devices using these techniques remain elusive. Regardless, widespread, routine screening using a PCR-based detection method is impractical.

Nanoscale materials, such as single wall carbon nanotubes (CNT) and gold (Au) nanoparticles are known. Furthermore, it is known how to functionalize gold nanoparticles with an oligonucleotide to detect DNA, such as in a lateral-flow dipstick. Also, a gold-nanoparticle-based staining technology was successfully used in genotyping single-nucleotide polymorphisms when combined with a primer extension reaction. A gold-nanowire microfluidics platform for sensitive detection of blood analytes is known, but this method requires an electrochemical device for the readout. None of these methods or materials have been combined with a rapid test kit for use in detecting a disease at the point of care.

### SUMMARY OF THE INVENTION

A rapid test for detecting infection is capable of detecting HIV infection in less than five minutes, and examples of test kits may be used for detecting the presence of antibodies or sequences of RNA or DNA in bodily fluids or both. In one example, a single test kit tests for both the presence of antibodies and a viral RNA sequence indicative of a disease. In an alternative example, separate test kits are provided for antibody screening and detecting a sequence or sequences of a particular DNA or RNA of a disease using a genetic probe.

Thus, a test kit using a genetic probe may be used in testing separately from a test kit for use in measuring antibodies presence in a sample of bodily fluids. In one example, only if a subject specimen tests positive for antibodies is a test kit including a genetic probe used for testing for a disease. The tests may be used to qualitatively and/or quantitatively determine a level or concentration of antibodies or sequences of RNA or DNA within the bodily fluid.

In one example, a comparison is made against a contrast scale to determine the relative level or concentration of detected antibodies and/or sequences of RNA or DNA in the type of bodily fluid tested. A quick indication of the presence of certain sequences of RNA or DNA in bodily fluids tested, may provide detection and/or confirmation of an infection, for example, especially an acute infection, such as in the case of an HIV infection.

In some examples, test kits have comparatively low flow rates and large particle retention size (correlating with pore size) and are capable of completion of a rapid test in less than 3 minutes. In one example, a test kit uses an antigen or a combination of antigens immobilized on a membrane, such as a cellulose filter paper, the membrane being selected to immobilize the antingen or antingens and having a flow rate in a range from about 0.04 ml/min/cm² to about 0.4 ml/min/cm². The test kit is capable of detecting antibodies by direct deposit, flow-through of a buffered suspension such as PBS buffered blood, serum or plasma, for example. None of the other tested commercial test kits were capable of testing whole blood, which was readily achieved using examples of the present invention without affecting the outcome and with similar contrast to the same test using serum or plasma. The prior art teaches that testing with whole blood is not known to achieve that same results as the use of serum or plasma. In one example, a particular portion of an antigen is used to improve the contrast of a positive indication region, especially for whole blood, and the short fragment of the antigen achieved better results than using the entire antigen.

In one example, a diagnostic kit includes an antigen-immobilizing cellulose filter paper, at least one antigen immobilized on the cellulose filter paper, a staining agent to detect antibodies against the at least one antigen, a destaining buffer to remove non-specific background staining, and a plurality of wicking layers disposed in a bottom portion of the diagnostic kit opposite of the reaction membrane. For example, a cellulose filter paper used as a reaction layer of the test kit may have a particle retention size selected in a range from about 6 to about 25 microns. Furthermore, test results for a variety of particle retention sizes for cellulose filter papers show that papers having particle retention sizes of 6, 11, and 20-25 (Whatman Qualitative/Wet Strengthened grade cellulose filter papers) do not exhibit a large departure in flow rate. A rapid test kit should not have a flow rate unnecessarily low, but there is a correlation between flow rate and a color index value reported in the results, which is related to sensitivity of the test kit for detecting antibodies. Thus, there is a preferred range for selecting cellulose filter paper with an optimum flow rate.

In one example, a staining agent is Protein A coupled to colloidal gold. A destaining buffer is used, such as phosphate buffered saline (PBS) to improve contrast with the background. In another example, a rapid test for detecting infection selects cellulose filter paper or an equivalent that has a phosphate buffer saline (PBS) solution flow rate in a range between about 0.04 to about 0.4 ml/min/cm², more preferably 0.04 to 0.2 ml/min/cm² for higher contrast (sensitivity). Flow rate, is more important than pore size in determining assay sensitivity and time to complete the test. In one embodiment, a cellulose filter paper can be selected to have a flow rate in a range from about 0.1 to about 0.2 ml/min/cm², providing an optimum trade-off in sensitivity and flow rate for some examples.

In another example, the cellulose filter paper can be selected to have a PBS flow rate in a range from about 0.2±0.05 ml/min/cm² to increase flow rate without unduly sacrificing sensitivity (i.e., color index value). In flow rate measurements, the term “about” is used to indicate the manufacturing variances in manufacturing cellulose filter paper and in testing of flow rate according to the modified ASTM method described herein. A person of ordinary skill in the art will be able to measure flow rates and select cellulose filter papers based on the disclosed flow rate testing method and flow rates and those cellulose filter papers having about the same flow rates as the ranges given herein.

One advantage of the diagnostic kit using cellulose as a reaction layer is the ability to obtain rapid results for a particular infectious agent or a plurality of infectious agents without complicated user protocols. Indeed, results are provided as readily for whole blood as for serum or plasma in some examples.

Another advantage is the cost of a test kit, which substantially reduces the costs associated with screening. A rapid test kit is inexpensively produced and provided at low cost, which is especially necessary for use in remote locations and doctor's offices. A single test may be used to test more than one type of disease detectable from blood.

Yet another advantage is that a single diagnostic kit may be used in detecting one or more of a variety of bodily fluids, such as blood, plasma and serum, thus offering greater flexibility in testing. Field tests may be administered without the need of a mobile laboratory or a centrifuge.

Yet another advantage is that the rapid test kit provides a rapid result and both good sensitivity and good specificity. In one example, a genetic probe is included for detecting RNA, DNA or a fragment or sequence of RNA or DNA in a bodily fluid. The probe may include a pair of primers, for example. One of the pair of primers may be immobilized on filter paper, while the other of the pair of primers is coupled to a nanoparticle or nanotube, such as by a thiolation of the other of the pair of primers, and the coupled primer-nanoparicle or primer-nanotube is included in a staining buffer. One advantage of the use of a genetic probe is that the genetic probe may provide a qualitive or quantitative analysis of the level or concentration of a sequence of RNA and/or DNA in the volume of a fluid tested, such as urine, blood, edema or saliva, which may be correlated to a viral load, for example. Another advantage is that the genetic probe may distinguish a vaccinated subject or a subject having maternal antibodies from a subject infected by a disease.

A rapid test kit for detection of a DNA, an RNA or a fragment of a DNA or an RNA includes a detection surface, such as a membrane, one or more genetic probes immobilized on a test portion of the detection surface, and a staining agent. The genetic probe is selected to hybridize a genetic sequence to be detected by the test, such as the genetic sequence of viral RNA, for example. The staining agent may include a different genetic probe, such as functionalized nanoparticle or nanotube that binds to the genetic sequence to be detected by the test. Thus, the genetic sequence is immobilized preferentially on a region of the detection surface, when a sample containing the genetic sequence is applied to the detection surface or is passed through the detection surface, such as a cellulose filter paper membrane. Then, the staining agent is immobilized by binding to the genetic sequence, providing a contrast between the test portion and a background portion of the membrane.

In one example, a destaining buffer may be selected to remove at least a portion of any non-specific background staining unrelated to binding between the at least one genetic probe, the DNA, RNA or the fragment of the DNA or the RNA, and the staining agent.

For example, the detection surface may be the surface of a glass slide or a membrane. The membrane may be a cellulose filter paper selected such that the membrane has a measured flow rate of a phosphate buffered saline from about 0.04 to about 0.4 mL/min/cm², using a modified ASTM standard flow rate measurement, for example. More preferably, the membrane may have a measured flow rate in a range from about 0.04 mL/min/cm² to about 0.2 mL/min/cm² in order to increase contrast between the test region and the background. The measured flow rate of the membrane may be limited to a range of at least 0.1 mL/min/cm² and no greater than about 0.2 mL/min/cm² in order to optimize the time required for testing and the contrast, for example.

In one example, the staining agent includes at least one type of oligonucleotide-functionalized nanoparticles or nanotubes having at least one oligonucliotide capable of hybridizing, at room temperature, with regions of genetic sequences to be detected by the rapid test kit.

Also, the at least one genetic probe may include a complimentary oligonucleotide for hybridization with a specific region of the genetic sequences to be detected by the kit. In one example, the complimentary oligonucleotide is conjugated with a chitosan or a chitosan derivative before being applied to the detection surface, such that the complimentary oligonucleotide is immobilized on a cellulose filter paper membrane, for example. Examples of chitosan and chitosan derivatives are provided in the prior art, such as a thiolated chitosan derivative in U.S. Pat. Publication US 2007/0036867, chitosan derivatives disclosed in U.S. Pat. Publication US 2008/0087290, and the like.

The oligonucleotide-functionalized nanoparticle or nanotube may be a gold nanoparticle functionalized by a thiolated oligonucleotide complementary to a different portion of the genetic sequence than the genetic sequence targeted by the complimentary oligonucleotide immobilized on the membrane, for example. The thiolated oligonucleotide may be a primer selected to hybridize a viral RNA selected from the group consisting of an HIV virus, a Hepatitis B virus, a Hepatitis C virus, a SARS virus and combinations thereof. For example, examples of oligonucleotides may be thiolated and bound to a surface of a gold nanoparticle, and may be selected to hybridize the viral RNA of the HIV virus. The complementary oligonucleotide immobilized on the membrane may be selected to hybridize the same or a different region of the viral RNA of the HIV virus, such as the LTR sequence, in one example. In an alternative example, the staining agent comprises carbon nanotubes functionalized by examples of the oligonucleotides.

Whether nanotubes or nanoparticles are used, the concentration may be adjusted to provide sufficient contrast under the appropriate lighting conditions in order to observe a positive test result. In one example, a plurality of oligonucleotides for attaching to a plurality of regions within a genetic sequence to be detected are provided in a staining agent. Each nanotube or nanoparticle may be functionalized with one or more of the oligonucleotides. Thus, a plurality of nanotubes or nanoparticles may be hybridized to one or more regions of a single genetic sequence, providing an amplification in the contrast observed compared to the use of only one oligonucleotide targeting one region of the single genetic sequence. On the other hand, the oligonucleotide or oligonucleotides immobilized on the detection surface may be targeted to only one or a select few regions of the genetic sequence in order to increase the specificity of the test kit to only the genetic sequence or sequences selected for detection. Tables 8A-P (intentionally omitting designators 8I and 8O for clarity) disclose a screening of genetic sequences for locating unique HIV specific genetic sequences that provide specificity for a rapid test, for example. By combining a specific complementary oligonucleotide on the detection surface and a plurality of oligonucleotides for binding each nanoparticle or nanotube to the genetic sequence, a test may have a very good contrast even with a low viral load, yet remain very selective in the genetic sequences detected, which provides a surprising and unexpected improvement over any known rapid test including the ability to distinguish between the effects of a vaccine on antibodies and a viral load, for example.

Furthermore, certain complementary oligonucleotides may be selected that hybridize with the selected regions of the genetic sequences. In a rapid test for use at point of care, it is preferred to select complementary oligonucleotides that hybridize at room temperature, for example. In this manner, specific oligonucleotides may be targeted for any genetic sequence, such as viral RNA's consisting of an HIV virus, a Hepatitis B virus, a Hepatitis C virus, a SARS virus and combinations thereof, for example. In one example, at least one genetic probe includes a 33-nt oligonucleotide from HIV 89.6 proviral clone, which may be conjugated with a chitosan or a chitosan derivative.

In one method, the method includes detecting a detectable level of viral load in a sample volume of fluid. The method may include a step of conjugating the at least one genetic probe with a chitosan or a chitosan derivative to form a conjugate, and immobilizing the conjugate on a test region of a membrane. In one example, the method includes illuminating the membrane with ultraviolet light to increase the contrast between the test portion and a background portion of the membrane, such that the ultraviolet light causes the test portion to fluoresce. A level or concentration of the viral load may be determined by comparing the contrast or fluorescence to known levels or concentrations, for example. In one example, the determination is automated by a detector and processor that compares the signal received by the detector to a look up table, for example. The step of reporting may include comparing the contrast or intensity of at least a portion of the test portion of the membrane to a standard, for example. In one method, a staining agent is deposited on the membrane such that a genetic probe in the staining agent binds selectively to a portion of a genetic sequence within a temperature range, which may include room temperature. Room temperature is considered to be a range of temperature from about 15 degrees centigrade to about 25 degrees centigrade, for example.

In one example, a genetic probe is combined with an antibody test. The antibody test may comprise one or more peptide fragments, such as a gp41 peptide fragment comprising SEQ. ID. NO. 14, as follows: QLQARILAVERYLKDQQLLGIWGCSGKLICTTAVPWNAS.

In another example, at least one genetic probe is deposited on a detection region of a glass slide, a fluid to be tested is deposited on the detection region, and a suspension of nanotubes or particles is functionalized by a complementary oligonucleotide such that, when the suspension is directly deposited on the detection region of the glass slide, the complimentary oligonucleotide hybridize specific regions of a genetic sequence, if the genetic sequence is present on the detection region. For example, the genetic probe may be fixed on the surface of the slide before placing the fluid onto the surface of the slide. After a fixed period of time, the fluid may be rinsed from the surface. Then, the staining agent may be deposited on the detection region for a fixed period of time within a temperature range, such as room temperature. The staining agent may be rinsed away and the slide may be observed under light, such as an ultraviolet light, to detect a contrast between the detection region and a control region or a background region. Alternatively, the detector may observe the slide for the emission of light or for the absorbtion of light by the detection region. For example, functionalized carbon nanotubes, functionalized by complementary oligonucleotides, may be used detecting a fluorescence under ultraviolet light, and functionalized gold nanoparticles may be used for detection of light absorbed passing the light, such as ultraviolet light, through the detection region. Either fluorescence or phosphorescence may be detected, for example. The detector or system may be capable of reporting a value or outpuot associated with a viral load in the sample measured, for example.

In one example, a genetic probe includes a 33-nt oligonucleotide from HIV 89.6 proviral clone, for example. A genetic probe may be immobilized on one portion of a test kit, and an antigen for detecting an antibody may be immobilized on another portion of a test kit. In one example, the two portions are in the same testing window and react to the same sample of a bodily fluid. In an alternative example, the two portion are disposed in separate windows. One or more staining agents may be used, which may include functionalized nanotubes or nanoparticles, for example. In one example, a complementary-oligonucleotide-functionlized nanotube or oligonucleotide functionalized particle is provided. The genetic probe may be a complimentary oligonucleotide capable of hybridizing a genetic sequence to be detected, such as a portion of the LTR genetic sequence of the HIV-1 virus. The staining agent may comprise a plurality of thiolated oligonucleotides coupled with gold nanoparticles selected such that the plurality of thiolated oligonucleotides are each capable of hybridizing different portions of the RNA of the HIV-1 virus, for example.

Other unexpected advantages, uses and devices, and variations and combinations of these, are presented in the figures and examples of the detailed description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The drawings describe some examples of a rapid diagnostic kit and a method for preparing and using the diagnostic kit.

FIG. 1A illustrates an example of a cross section of a diagnostic kit 100.

FIG. 1B illustrates another example of a cross section of a diagnostic kit 110.

FIG. 1C depicts a top plan view of a diagnostic kit such as those shown in FIGS. 1A and 1B.

FIGS. 2A-B provide illustrations of top views of examples of test kits that (A) tested negative for the presence of an antibody and (B) tested positive for the presence of an antibody.

FIGS. 3A-B provide illustrations of a comparison of (B) an example of a diagnostic kit using a cellulose filter paper and (A) a glass fiber membrane, which resulted in failure when tested with blood.

FIGS. 4A-C illustrate comparisons of examples using a cellulose filter paper membrane for a diagnositc kit with a nitrocellulose membrane.

FIG. 5 graphs color index value versus flow rate of PBS, as measured using a modified ASTM flow rate procedure with 7 cm circles of the cellulose filter papers used in the tests.

FIG. 6 illustrates a color index chart for determining color index values where any marker discernable over background is given a value of 1, anything darker than 1 is 2, anything darker than 2 is 3, and anything darker than 3 is deemed a 4, quantifying color intensity of test samples.

FIG. 7 shows measured flow rate versus particle retention size for 6 different cellulose filter papers.

FIG. 8 discloses a graph color index value by sample number for various results including testes using blood and plasma with a test kit having a PBS flow rate of about 0.1 mil/min/cm², and also showing color index of control spots.

FIG. 9 is an illustration of test results using blood.

FIG. 10 graphically compares test results for plasma using a rapid test kit of the examples using a cellulose filter paper having a flow rate of about 0.1 ml/min/cm² and a commercially available test kit (Reveal® G3)¹. ¹ Reveal® is a registered trademark of MedMira Laboratories, Inc., Toronto, Canada.

FIG. 11 is an illustration of test results using whole blood.

FIG. 12 is an illustration of test results using plasma.

FIG. 13 is an illustration of test results using plasma.

FIGS. 14A-C illustrate possible outcomes of a test kit having a control test spot and an antibody test spot for both a genetic probe and antibodies with (A) two separate test windows; (B) a single test window; and (C) graphical representation of all outcome for the two test spots (i.e. assuming controls visible).

FIG. 15 illustrates an example of a process for using a rapid test kit including a genetic probe.

FIG. 16 illustrates, schematically, functionalization of a gold nanoparticle.

FIG. 17 illustrates a perception of contrast between (1) ssDNA-gold nanoparticles hybridized by complementary DNA; (2) ssDNA-gold nanoparticles with ssDNA; and (3) ssDNA-gold nanoparticles alone.

FIG. 18 graphs the ultraviolet absorbance spectra as shown and disclosed.

FIG. 19 illustrates, schematically, functionalization of a carbon nanotube.

FIG. 20 graphs ultraviolet light absorbance spetra for (A) carbon nanotubes (CNT), alone, without any ssDNA strand or fragment; (B) CNT functionalized by a single strand oligonucleotide; (C) CNT funtionalized by a single strand oligonucleotide hybridized with a non-complementary single strand oligonucleotide; and (D) CNT functionalized by a single strand nucleotide after hybridization with a complementary oligonucleotide fragment.

FIGS. 21A-21C are atomic force microscopy micrographs of (A) carbon nanotubes (CNT), alone, without any single strand oligonucleotides; (B) CNT functionalized by a single strand oligonucleotide; and (C) CNT functionalized by a single strand oligonucleotide and hybridization with a complementary single strand oligonucleotide fragment.

FIGS. 22A-B illustrate a control 784, 794 with a non-complementary 786, 796 and a complementary 788, 798 combination of single strand oligonucleotides (e.g. ssDNA or a fragment of ssDNA) for comparison. The regions having complementary 788, 798 single strand oligonucleotides fluoresce, while the non-complementary 786, 796 oligonucleotides fail to hybridize and are not subject to fluorescence.

FIG. 23 illustrates a range of concentrations of carbon nanotubes using a range of picomoles (pmol).

FIG. 24 contrasts a oligonucleotide that is not immobilized on a test region to an oligonucleotide immobilized on a test region after conjugating with a chitosan or a chitosan derivative.

FIG. 25 illustrates a detector for measuring emitted or transmitted light from a test region on a slide.

### DETAILED DESCRIPTION

The examples described and the drawings rendered are illustrative and are not to be read as limiting the scope of the invention as it is defined by the appended claims. Additional advantages of the invention shall be apparent to a person of ordinary skill from the description of the examples provided.

A rapid diagnostic assay provides a quick and inexpensive screening test for detecting antibodies resulting from disease-causing organisms, such as a viruses, bacteria, fungus, mold and other disease-causing organisms that are detectable through an antibody assay. The diagnostic assay is a rapid assay meaning that the time to conduct the test from drawing of a bodily fluid to completing the test is rapid (e.g. less than ten minutes) and the time to obtain a test result after preparing a buffered suspension is rapid (e.g. less than one minute). Rapid test kits are not known that have both the sensitivity and the specificity of test kits used in the examples. Furthermore, none of the test kits known to the inventors are able to provide a result in less than 1 minute from the time that PBS buffered samples are ready to be used, such as shown for test kits obtaining strong positives in high titer tests and excellent results in low titer tests, also. Rapid is meant to mean both time scales (test preparation to completion and time for the test kit to provide a result after the test sample is mixed in buffer solution). Furthermore, examples of test kits provide rapid diagnostic assay using whole blood, serum or plasma as testing material. Whole blood is particularly problematic for all of the commercial test kits tested.

In one example, a method of rapid diagnostic assay uses the test kit of the examples in the field without any need of medical or laboratory facilities. Ability to distribute to remote locations makes testing convenient and inexpensive.

Various types of antigens may be used in a rapid diagnostic assay. The antibodies detected by a rapid diagnostic assay may be produced in response to bacteria, fungi, parasites, or viruses, for example. A wide variety of antigens may be used separately or together in a screening array. In addition to infectious diseases, the rapid diagnostic assay may also detect antibodies or antigens in non-infectious diseases such as cancer, Alzheimer's disease, or other non-infectious diseases.

Bacterial antigens Bacterial pathogens may be detected by a rapid test kit. In one example, an antigen is selected from a major outer membrane protein within strains of the genus Actinobacillus. For example, the antigen is disclosed in U.S. Pat. No. 6,541,011. In another example, a bacterial antigen may be from any of the following: Actinomyces, such as an ornithine-rich antigen from Actinomyces naeslundii, or Actinomyces viscosus as disclosed in U.S. Pat. No. 6,974,700; Aerobacter aerogens or Actinomyces israelli; Bacillus, such as Bacillus anthracis or Bacillus cereus or Bacillus subtilis or a protective antigen, lethal factor or an edema factor of Bacillus anthracis, as disclosed in WO 2004/024067; cell surface antigens of B. cereus as disclosed in U.S. Pat. No. 6,699,679; a 69 Kd protein of B. pertussis as disclosed in U.S. Pat. No. 5,527,529; Bacteroides; Bordetella; B. pertussis, such as mentioned in U.S. Pat. No. 6,197,548; B. parapertussis and B. bronchiseptica with molecular masses of 70 and 68 kDa respectively; Bartonella; Borrelia, such as Borrelia recurrentis or OspA of the Lyme disease Borrelia burgdorferi, as mentioned in U.S. Pat. No. 6,541,011; Brucella, such as Brucella abortus or Brucella melitensis, such as Omp29 on Brucella melitensis as mentioned in U.S. Pat. No. 6,541,011 or Brucella suis; Campylobacter, such as Campylobacter pylori as mentioned in U.S. Pat. No. 5,549,051; Capnocytophaga; Chlamydia, such as Chlamydia traqchomatis or Chlamydia psittaci, such as 80-90 kDa protein and 110 kDa protein, chlamydial exoglycolipid (GLXA), Chlamydia pneumoniae species-specific antigens in the molecular weight ranges 92-98, 51-55, 43-46 and 31.5-33 kDa and genus-specific antigens in the ranges 12, 26 and 65-70 kDa, as mentioned in U.S. Pat. No. 6,541,011; Clostridium, such as Clostridium botulinum or Clostridium perfingens or Clostridium tetani or a C fragment from C. tetani as mentioned in U.S. Pat. No. 5,527,529; A, B, C, and D toxoids from C. perfringens, such as a B toxoid as mentioned in U.S. Pat. No. 6,524,592 or toxin A from C. difficile, as mentioned in U.S. Pat. No. 6,503,722 or LT and HT toxins from C. sordellii disclosed in U.S. Pat. No. 6,849,715 or an alpha toxin from C. septicum of U.S. Pat. No. 7,037,503 or A-G toxins from C. botulinum as disclosed in U.S. Pat. No. 6,613,329; Corynebacterium, such as Corynebacterium diptheria; Coxiella; Dermatophilus; Enterococcus; Ehrlichia; Echinococcus granulosus antigen 5, as disclosed in U.S. Pat. No. 6,541,011; Escherichia coli; Francisella; Fusobacteria; H. pylori, such H. pylori GroES homologue (HspA) and four immunoreaction proteins of 45-65 kDa, as mentioned in U.S. Pat. No. 6,541,011; Hemophilus influenzae; H. ducreyi; H. hemophilus; H. aegypticus; H. parainfluenzae; Haemobartonella; Helicobacter; Klebsiella, such as the K antigen of Klebsiella pneumonia, disclosed in U.S. Pat. No. 6,541,011; Leptospira icterohemorrhagiae, such as Leptospira canicola; Leishmania, such as gp63 of Leishmania major, disclosed in U.S. Pat. No. 6,541,011; mycobacterial heat shock protein 65, disclosed in U.S. Pat. No. 6,541,011; Leptospira; Listeria, such as Listeriolysin O of Listeria monocytogenes as disclosed in U.S. Pat. No. 5,830,702; Moraxella, such as the CD protein of Moraxella, mentioned in U.S. Pat. No. 6,541,011; Mycobacteria, such as Mycobacterium avium or Mycobacterium bovis or Mycobacterium leprae or Mycobacterium paratuberculosis or Mycobacterium tuberculosis hominis or a 35 kilodalton protein of Mycobacterium leprae, as disclosed in U.S. Pat. No. 6,541,011, or 85 or 45/47 kDa antigen of Mycobacterium tuberculosis, as disclosed in U.S. Pat. No. 6,541,011 or 18-kilodalton protein of Mycobacterium lepraes, as disclosed in U.S. Pat. No. 6,541,011; Mycoplasma. In one example, the antigen is from Mycoplasma hominis. In one example, the antigen is from Mycoplasma pneumoniae.

In one example, the bacterial antigen is from Neisseria. In one example, the antigen is from Neisseria gonorrhea. In one example, the antigen is from Neisseria meningitidis. In one specific example, the antigen is Por, Rmp or a LOS protein of Neisseria gonorrhoeae. In another example, the antigen may include PorA, Por B, Rmp, Opc, FrpB, TbpB or Nsp may be used, as mentioned by U.S. Pat. No. 6,797,273; Neorickettsia; Nocardia; Pasteurella, such as Pasteurella pestis; Peptococcus, such as Peptostreptococcus; Pneumococcus, such as Diplococcus pneumonia; Proteus; Pseudomonas; P. gingivalis, such as the 43-kDa and the fimbrilin (41 kDa) proteins of P. gingivalis, as disclosed in U.S. Pat. No. 6,541,011; Rickettsia, such as Rickettsia australis or Rickettsia burneill or Rickettsia conori or Rickettsia mooseri or Rickettsia prowazekii or Rickettsia tsutsugamushi; Rochalimaea; Salmonella, such as Salmonella choleraesus or Salmonella typhimurium or Salmonella typhosa or O, H, and Vi antigens of Salmonella or SEF14 fibrial antigen of Salmonella enteriditis and flagellar (G) antigens observed on Salmonella enteritidis and S. pullorum, disclosed in U.S. Pat. No. 6,541,011; Shigella, such as Shigella arabinotardo or Shigella boydii or Shigella dysenteria or Shigella flexneri or Shigella schmitzii or Shigella sonnei or O-antigens disclosed by U.S. Pat. No. 5,958,686 or S. dysenteria, disclosed in U.S. Pat. No. 5,204,097; Staphylococcus, such as Staphylococcus aureus or Staphylococcus albus or type 5, type 336, type 4, K73 antigens of S. aureus, disclosed by U.S. Pat. No. 6,537,559; hyperimmune serum reactive antigen of S. epidermidis, as suggested by U.S. Patent Publication 2007/0036778; ORF-2 antigen of Staphylococcus aureus or GlpQ, each disclosed in U.S. Pat. No. 6,541,011; Streptococcus, such as Streptococcus agalactiae (Group B Streptococcus), Streptococcus (viridans group), Streptococcus faecalis, Streptococcus bovis, Streptococcus (anaerobic sps.), or Streptococcus pneumoniae, as disclosed in U.S. Pat. No. 6,429,199 or 190 kDa protein antigen of Streptococcus mutans discussed in U.S. Pat. No. 6,541,011; M proteins or C5a peptidase of Streptococcus pyogenes disclosed in WO 2002/050107; other examples of streptococcus pyogenes include group carbohydrate antigen, C-substance, fimbrial proteins, fibronectin-binding proteins (e.g., Protein F), a cell bound streptokinase, A, B, and C streptococcal pyrogenic exotoxins, alpha C protein, beta C protein, Rib and Sip proteins, or group B carbohydrate antigens, as disclosed in U.S. Patent Publication 2006/0269541; purified capsular polysaccharide of 7 serotypes of S. pneumoniae (4.9V, 14, 19F, 23F, 18 C and 6B); pneumococcal surface protein A, pneumococcal surface adhesion A, choline binding protein A, LytB glucosaminidase, LytC muramidase, PrtA serine protease, PhtA (histidine triad A) and pneumococcal vaccine antigen A, as mentioned in WO/2004/092209; Group B streptococcal Ema (extracellular matrix adhesion protein polypeptides) EmaA, EmaB, EmaC, EmaD and EmaE from U.S. Pat. No. 7,128,919; Pac antigen of Streptococcus mutans, as mentioned in U.S. Pat. No. 6,541,011; MW antigens of Salmonella typhi, mentioned in U.S. Pat. No. 6,541,011; Treponema pallidum; Yersinia, such as V antigen or F1 antigen or pH6 antigen of Yersina pestis, disclosed in U.S. Pat. No. 6,541,011 or 37 kDa secreted polypeptide encoded on the 70 kb virulence plasmid of pathogenic Yersinia as disclosed in U.S. Pat. No. 6,541,011.

Fungal antigens Fungal pathogens may also be detected by the kit and the methods disclosed. In one example, the antigen is from Candida albicans. In one specific example, the antigen is a fungal adhesion molecule, such as a phosphomannoprotein, from Candida albicans, as mentioned in U.S. Pat. No. 6,630,146. In one example, the antigen is a fungal antigen from Absidia. In one example, the antigen is from Absidia corymbifera. In one example, the antigen is a fungal antigen from Acremonium. In one example, the antigen is a fungal antigen from Alternaria. In one example, the antigen is a fungal antigen from Aspergillus. In one example, the antigen is a fungal antigen from the species Basidiobolus. In one example, the antigen is a fungal antigen from the species Bipolaris. In one example, the antigen is a fungal antigen from the species Blastomyces. In one example, the antigen is a fungal antigen from the species Blastomyces. In one example, the antigen is a fungal antigen from Candida. One specific example of an antigen is a fungal adhesion molecule, such as a phosphomannoprotein, from Candida albicans, as mentioned in U.S. Pat. No. 6,630,146. In one example, the antigen is a fungal antigen from Candida. In one example, the antigen is a fungal antigen from Coccidioides. In one example, the antigen is from Coccidioides immitis. In one example, the antigen is a fungal antigen from Conidiobolus. In one example, the antigen is a fungal antigen from Cryptococcus. In one example, the antigen is a fungal antigen from Conidiobolus. In one example, the antigen is a fungal antigen from Cryptococcus. In one example, the antigen is from Cryptococcus neoformans. In one example, the antigen is a fungal antigen from Curvalaria. In one example, the antigen is a fungal antigen from Epidermophyton. In one example, the antigen is a fungal antigen from Exophiala. In one example, the antigen is a fungal antigen from Geotrichum. In one example, the antigen is a fungal antigen from Histoplasma. In one example, the antigen is from Histoplasma capsulatum. In one example, the antigen is a fungal antigen from Madurella. In one example, the antigen is a fungal antigen from Malassezia. In one example, the antigen is a fungal antigen from Microsporum. In one example, the antigen is a fungal antigen from Moniliella. In one example, the antigen is a fungal antigen from Mortierella. In one example, the antigen is a fungal antigen from Mucor. In one example, the antigen is a fungal antigen from Paecilomyces. In one example, the antigen is a fungal antigen from Penicillium. In one example, the antigen is a fungal antigen from Phialemonium. In one example, the antigen is a fungal antigen from Phialophora. In one example, the antigen is a fungal antigen from Prototheca. In one example, the antigen is a fungal antigen from Pseudallescheria. In one example, the antigen is a fungal antigen from Pseudomicrodochium. In one example, the antigen is a fungal antigen from Pythium. In one example, the antigen is a fungal antigen from Rhinosporidium. In one example, the antigen is a fungal antigen from Rhizopus. In one example, the antigen is a fungal antigen from Scolecobasidium. In one example, the antigen is a fungal antigen from Sporothrix. In one example, the antigen is a fungal antigen from Stemphylium. In one example, the antigen is a fungal antigen from Trichophyton. In one example, the antigen is a fungal antigen from Trichosporon. In one example, the antigen is a fungal antigen from Xylohypha.

Parasital antigens Parasital pathogens may also be detected by the kit and the methods disclosed. In one example, the antigen is a protozoan parasite and the antigen is from Babesia. In one example, the antigen is a protozoan parasite and the antigen is from Balantidium. In one example, the antigen is a protozoan parasite and the antigen is from Balantidium. In one example, the antigen is a protozoan parasite and the antigen is from Besnoitia. In one example, the antigen is a protozoan parasite and the antigen is from Cryptosporidium. In one example, the antigen is a protozoan parasite and the antigen is from Eimeria. In one example, the antigen is a protozoan parasite and the antigen is from Encephalitozoon. In one example, the antigen is a protozoan parasite and the antigen is from Entamoeba. In one example, the antigen is a protozoan parasite and the antigen is from Giardia. In one example, the antigen is a protozoan parasite and the antigen is from Hammondia. In one example, the antigen is a protozoan parasite and the antigen is from Hepatozoon. In one example, the antigen is a protozoan parasite and the antigen is from Isospora. In one example, the antigen is a protozoan parasite and the antigen is from Leishmania. In one example, the antigen is a protozoan parasite and the antigen is from Microsporidia. In one example, the antigen is a protozoan parasite and the antigen is from Neospora. In one example, the antigen is a protozoan parasite and the antigen is from Neospora. In one example, the antigen is a protozoan parasite and the antigen is from Pentatrichomonas. In one example, the antigen is a protozoan parasite and the antigen is from Plasmodium. In one example, the antigen is a protozoan parasite and the antigen is from Plasmodium. In specific examples, the antigens may include P. falciparum circumsporozoite (PfCSP), sporozoite surface protein 2 (PfSSP2), carboxyl terminus of liver state antigen 1 (PfLSA1 c-term), and exported protein 1 (PfExp-1). In one example, the antigen is from a protozoan parasite Pneumocystis. In one example, the antigen is from a protozoan parasite Sarcocystis. In one example, the antigen is from a protozoan parasite Schistosoma. In one example, the antigen is from a protozoan parasite Theileria. In one example, the antigen is from a protozoan parasite Toxoplasma. In one example, the antigen is from a protozoan parasite Trypanosoma. In other examples, the antigen is from helminth parasites. In one example, the antigen is from Acanthocheilonema. In one example, the antigen is from Aelurostrongylus. In one example, the antigen is from Ancylostoma. In one example, the antigen is from Angiostrongylus. In one example, the antigen is from Ascaris. In one example, the antigen is from Brugia. In one example, the antigen is from Bunostomum. In one example, the antigen is from Capillaria. In one example, the antigen is from Chabertia. In one example, the antigen is from Cooperia. In one example, the antigen is from Cooperia. In one example, the antigen is from Crenosoma. In one example, the antigen is from Dictyocaulus. In one example, the antigen is from Dioctophyme. In one example, the antigen is from Dipetalonema. In one example, the antigen is from Diphyllobothrium. In one example, the antigen is from Diplydium. In one example, the antigen is from Dirofilaria. In one example, the antigen is from Dracunculus. In one example, the antigen is from Enterobius. In one example, the antigen is from Filaroides. In one example, the antigen is from Haemonchus. In one example, the antigen is from Lagochilascaris. In one example, the antigen is from Loa. In one example, the antigen is from Mansonella. In one example, the antigen is from Muellerius. In one example, the antigen is from Nanophyetus. In one example, the antigen is from Necator. In one example, the antigen is from Nematodirus. In one example, the antigen is from Oesophagostomum. In one example, the antigen is from Onchocerca. In one example, the antigen is from Opisthorchis. In one example, the antigen is from Ostertagia. In one example, the antigen is from Parafilaria. In one example, the antigen is from Paragonimus. In one example, the antigen is from Parascaris. In one example, the antigen is from Physaloptera. In one example, the antigen is from Protostrongylus. In one example, the antigen is from Setaria. In one example, the antigen is from Spirocerca. In one example, the antigen is from Spirometra. In one example, the antigen is from Stephanofilaria. In one example, the antigen is from Strongyloides. In one example, the antigen is from Strongylus. In one example, the antigen is from Thelazia. In one example, the antigen is from Toxascaris. In one example, the antigen is from Toxocara. In one example, the antigen is from Trichinella. In one example, the antigen is from Trichostrongylus. In one example, the antigen is from Trichuris. In one example, the antigen is from Uncinaria. In one example, the antigen is from Wuchereria. In one example, the antigen may include the schistosome gut-associated antigens CAA (circulating anodic antigen) and CCA (circulating cathodic antigen) in Schistosoma mansoni, S. haematobium or S. japonicum. In one example, the antigen may include a multiple antigen peptide (MAP) composed of two distinct protective antigens derived from the parasite Schistosoma mansoni. In one example, the antigen may include Leishmania parasite surface molecules third-stage larval (L3) antigens of L. loa (Akue et al. (1997), Tams1-1 and Tams1-2, encoding the 30- and 32-kDa major merozoite surface antigens of Theileria annulata (Ta) and Plasmodium falciparum merozoite surface antigen 1 or 2. In one example, the antigen is Plasimodium falciparum antigen Pfs230. In one example, the antigen may include Plasimodium falciparum apical membrane antigen (AMA-I); Plasmodium falciparum proteins Pfs28 and Pfs25; Plasimodium falciparum merozoite surface protein, MSP1; the malaria antigen Pf332; Plasmodium falciparum erythrocyte membrane protein 1; Plasmodium falciparum merozoite surface antigen, PfMSP-1; Plasmodium falciparum antigens SERA, EBA-175, RAP1 and RAP2; Schistosoma japonicum paramyosin (Sj97) or fragments; and Hsp70 in parasites.

Viral antigens Viral pathogens may also be detected by the kit and the methods disclosed. In one example, the antigen is a viral antigen from an adenovirus. In one example, the antigen is a viral antigen from an alphavirus. In one example, the antigen is a viral antigen from a calicivirus. In one example, the antigen is a viral antigen from a calicivirus capsid antigen. In one example, the antigen is a viral antigen from a coronavirus. In a specific example of a coronavirus, the antigen is a SARS coronavirus. In one example, the antigen is from a cytomegalovirus. In one specific example, the antigen may include cytomegalovirus glycoprotein gB or glycoprotein gH. In one example, the antigen is a Dengue virus. In one specific example, the antigen may include a Dengue virus envelope (E) and premembrane antigens. In one example, the antigen is a viral antigen from a distemper virus. In one example, the antigen is a viral antigen from an Ebola virus. In one example, the antigen is from an Epstein-Barr virus. In one specific example, the antigen is an Epstein-Barr virus (EBV) gp340 protein. In another specific example, the antigen is the Epstein-Barr virus (EBV) latent membrane protein LMP2.

In one example, the antigen is Epstein-Barr virus nuclear antigens 1 and 2. In one example, the antigen is measles virus nucleoprotein (N). In one example, the antigen is a viral antigen from an enterovirus. In one example, the antigen is a viral antigen from a flavivirus. In one example, the antigen is from Hepatitis A. In one example, the antigen is from Hepatitis B. In one example, the antigen is a viral antigen from a hepatitis B core or surface antigen. In one specific example, the antigen is Hepatitis B virus core and E antigen. In one specific example, the antigen is a hepatitis B surface antigen fused to a core antigen, core-preS2 particles. In one example, the antigen is from Hepatitis C. In one specific example, the antigen is a Hepatitis C virus nucleocapsid protein in a secreted or a nonsecreted form. In another specific example, the antigens may include the hepatitis C virus antigens: the core protein (pC); E1 (pE1) and E2 (pE2) alone or as fusion proteins. In one example, the antigen is from Herpes simplex, types I and II. In one example, the antigen is a viral antigen from a herpes simplex virus or varicella zoster virus glycoprotein. In one specific example, the antigen may include ICP0, ICP4, ICP27, ICP47, gB, gD, gE, gG, gH, and gI of the herpes simplex virus. In one example, the antigen is a viral antigen from an infectious peritonitis virus. In one example, the antigen is a viral antigen from HIV. In one specific example, the antigen may include a HIV antigen such as Gag, Pol, Vif, Nef, p24, gp120, gp 160, gp41 or gp36. In one example, the antigen is a viral antigen from an influenza virus. In one example, the antigen is from an influenza A, B or C viruses. In one specific example, the antigen is a viral antigen from an influenza A hemagglutinin, neuraminidase, or nucleoprotein. In one specific example, the antigen is N2 neuraminidase of an influenza A virus. In one example, the antigen is a viral antigen from a leukemia virus. In one example, the antigen is a viral antigen from a Marburg virus. In one example, the antigen is from a measles virus. In one example, the antigen is from the mumps virus. In one example, the antigen is a viral antigen from an orthomyxovirus. In one example, the antigen is a viral antigen from a papilloma virus. In one specific example the antigen may include the E1, E2, E3, E4, E5, E6 and E7 proteins of human papillomavirus. In one example, the antigen is a viral antigen from a parainfluenza virus. In one specific example of a viral antigen from a parainfluenza virus, the antigen is a hemagglutinin or a neuraminidase. In one example, the antigen is a viral antigen from a paramyxovirus. In one example, the antigen is a viral antigen from a pestivirus. In one example, the antigen is a viral antigen from a picorna virus. In an example of a picornavirus, the antigen may come from a coxsackievirus. In an example of a picornavirus, the antigen may come from an echovirus. In an example of a picornavirus, the antigen may come from a poliovirus. In an example of a picornavirus, the antigen may come from a rhinovirus. In one specific example of a picorna virus antigens, the antigens may include a poliovirus capsid antigen, or a pox virus antigen. In one example, the antigen is a viral antigen from a rabies virus. In one specific example, the antigens include rabies virus glycoproteins. In one example, the antigen is a viral antigen from a reovirus. In one example, the antigen is from a respiratory syncytial virus. In one specific example, the antigen is a respiratory syncytial virus fusion protein (PFP-2). In one example, the antigen is from a rubella virus. In one example, the antigen is a viral antigen from a rotavirus. In one specific example, the antigen may include rotavirus antigen VP4, VP7, or VP7sc. In another specific example, the antigen may include proteins encoded by the VP6 and VP7 genes of rotaviruses. In one example, the antigen may be from vaccinia. In one example, the antigen is from human T-lymphotropic virus. In one specific example, the antigen may include a human T-lymphotropic virus type I gag protein.

In one example, an antigen is selected to detect a non-infectious disease, such as cancer, Alzheimer's disease or other non-infectious diseases. For example, the cancer may be prostate cancer, and the antigen selected may be a prostate specific antigen (PSA). In an example of antigen from Alzheimer's disease, the antigen is an Alzheimer's disease antigen, i.e., A68, or a recombinant human tau, as described in U.S. Pat. No. 6,864,062, for example.

### EXAMPLES

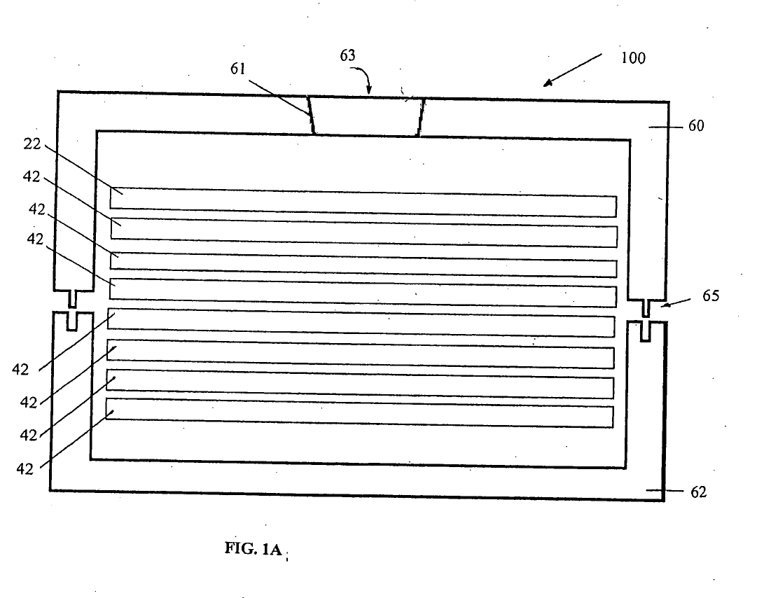

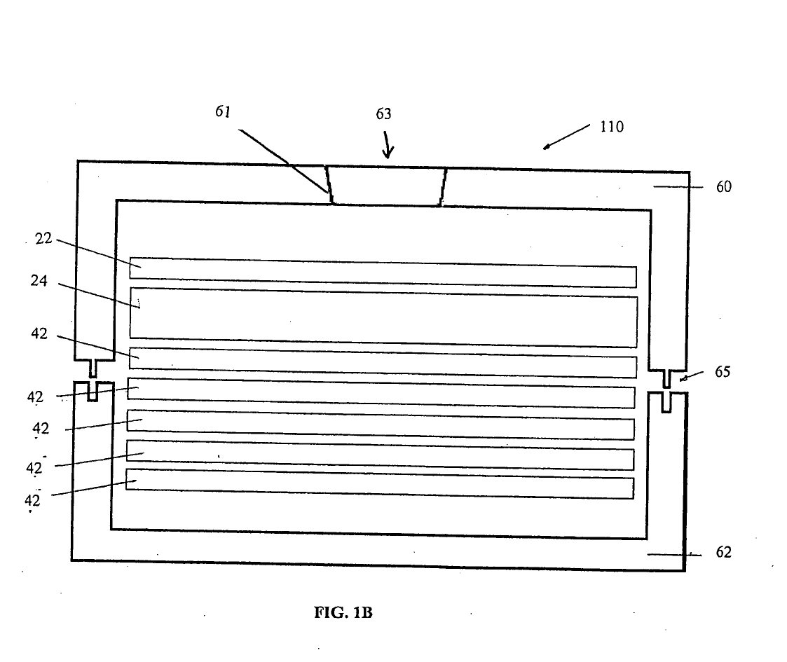

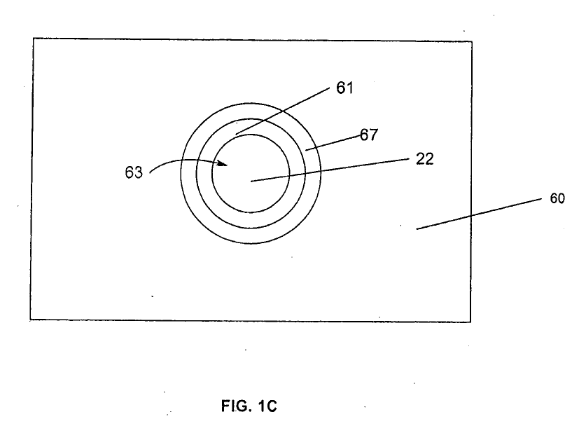

FIGS. 1A-C show a schematic example of a test kit assembly cross section. A plurality of layers 42 of an absorbent material and a membrane 22 are compressed between a cassette top 60 and a cassette bottom 62, which are represented in the drawin in an exploded view, for clarity.

As shown in FIG. 1A, a rapid test kit 100 comprises a cassette top 60 having an opening 63 and a cassette bottom 62. A wall 61 of port 63 may be angled or may be straight as shown. Additionally depicted is connection part 65, which may provide a snap or press fit, for example. A cellulose filter paper 22 may be loaded with one or more antigens. A plurality of absorbent layers 42 may be the same as the filter paper 22 or may be different. The absorbent layers 42 may have the same physical and chemical characteristics or may differ from each other, including length, absorbancy and thickness. In one example of the filter paper 22 and plurality of absorbant layers 42 have a dimension of 1 inch squares. The layers may be of uneven length, width and thickness. The plurality of absorbant layers 42 may be two or more depending on their thickness and the dimensions of the cavity formed by the top 60 and the bottom 62. Preferably, the top 60 and the bottom 62 compress the layers 42 to achieve intimate physical contact one to the other. In one example, the layers 42 are of a filter paper and include five to ten layers, depending on the characteristics of the filter paper and the cassette.

For example, a cassette top 60 may be press or snap fitted onto the cassette bottom 62. A central opening or port 63, through which plasma, serum, blood, saliva or other body fluids pass through the device, includes antigens for detecting antibodies. The antigen or antigens, may be loaded before testing either before or after assembly of the kit. In FIG. 1B, a wicking pad 24 replaces one or more absorbant layers 42 of a test kit 110.

In FIG. 1C, a top plan view of a diagnostic kit is illustrated. The cassette top 60 includes an angular wall 61 defining a port 63. The length of the wall 61 may be increased by a collar 67 extending above the top 60 and providing a greater volume within the port 63.

In one example 1 μl of an antigen or antigen mixture is added at a position T (i.e., a test position) of the flow through device and 1 μl of protein A (1 mg/ml) is added at a different position C (i.e., a control position) of the test device. Then, the test device is dried. For example, 6-8 hours of air drying is sufficient for drying most test kits. A test sample, such as blood, serum or plasma, may be tested for presence of an antibody using a staining buffer. In one example, the staining buffer is Protein A coupled to colloidal gold. For example, a staining buffer may be freeze-dried for later use and may be rehydrated using a buffer solution, such as 1× Dulbecco's Phosphate Buffer Saline (DPBS), for example.

For detection of antibodies specific to a given antigen, for example, 10 μl of serum, plasma, or whole blood of a test sample may be diluted with 150 μl of dilution buffer. In one example, the dilution buffer is ACK Lysis Buffer, Cat # 1683, obtained from Invitrogen. The now diluted sample is deposited into a port 63 of a test device and onto the reaction layer 22, which may be comprised of an antigen test spot on a cellulose filter paper. For a blood sample, it is advised to wait for about three minutes after the blood is added to the dilution buffer, or at least until the dilution buffer becomes uniformly a clear red. Once the diluted sample is absorbed, 150 μl of a staining buffer may be added. In one example, the staining buffer is Protein A coupled to colloidal gold. Once the staining buffer is absorbed, 200 μl of destaining buffer may be added. The destaining buffer may be Dulbecco's Phosphate Buffer (1×) Saline (DPBS), which is also an example of phosphate buffer solution, for example. Once the destaining buffer flushes the system, results may be read immediately, without further delay, resulting in a rapid test. When both test position T and control position C appear red, a test result is positive for the presence of antibodies indicative of a particular disease, such as HIV, for example. However, if only the control position C has a red dot, then the test result is negative for the presence of antibodies associated with the disease detected by the antigen. If no dot is visible or if the control position has no dot visible, then the test is invalid. The control dot C should always be visible, if the test is properly performed.

In one example of the process, a blood sample is diluted ten fold with a lysing buffer. Samples testing positive for a specific antibody have two red dots. In another example of the process, a silver enhancing buffer is used to improve contrast.

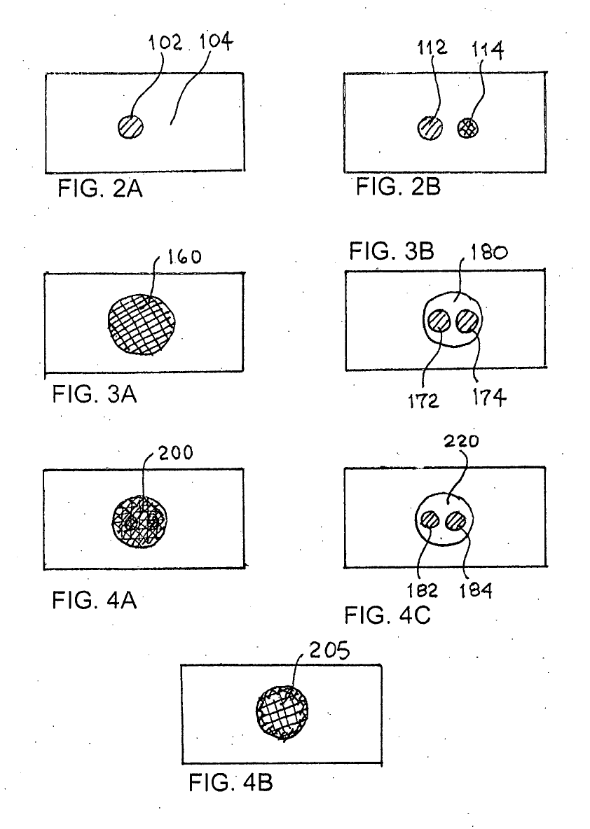

For example, as illustrated schematically in FIG. 2, a first C spot 102 of a first test device 120 and second C spot 112 of a second test device 140 serve as control spots, which help to confirm that the test device is functioning properly. A first T spot 104 of the first test device 120 and a second T spot 114 of a second device 140 are test spots for detecting the presence of a specific antibody or antibodies. None of the tests performed resulted in false negatives.

In the example of FIG. 2A, the first T spot 104 has no red spot, indicating the absence of any detectable level of antibodies in the particular test sample. In the example of FIG. 2B the T spot 114, shows a red spot in addition to control spot 112, positively indicating infection of the specimen with antibodies for HIV.

The following examples illustrate various types of antigens that may be used in a rapid test kit. An antibody or antibodies present in a sample may bind to the specific antigen. The examples are not intended to limit the type of antibody tested by the test kit, as any antibody that is capable of being tested in bodily fluid, such as blood, serum or plasma or other bodily fluids may be tested.

### Example 1

### Actinomyces

In one example, the antigen is Actinomyces. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 2

### Aerobacter Aerogens

In one example, the antigen is Aerobacter aerogens. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 3

### Bacillus

In one example, the antigen is Bacillus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 4

### Bacteroides

In one example, the antigen is Bacteroides. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 5

### Bartonella

In one example, the antigen is from the species Bartonella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 6

### Borrelia

In one example, the antigen is selected from a species of Borrelia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 7

### Brucella

In one example, the antigen is selected from a species of Brucella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 8

### Campylobacter

In one example, the antigen is selected from a species of Campylobacter or detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 9

### Chlamydia

In one example, the antigen is selected from a species of Chlamydia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 10

### Clostridium

In one example, the antigen is selected from a species of Clostridium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 11

### Corynebacterium

In one example, the antigen is selected from a species of Corynebacterium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 12

### H. pylori

In one example, the antigen is selected to be H. pylori. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 13

### Helicobacter

In one example, the antigen is selected to be Heliobacter. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 14

### Hemophilus influenzae

In one example, the antigen is selected to be Hemophilus influenzae. For detection of an antibody or antibodies specific to the antigen, 10 of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 15

### Klebsiella

In one example, the antigen is selected to be from a species of Klebsiella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 16

### Leptospira icterohemorrhagiae

In one example, the antigen is selected to be from a species of Leptospira icterohemorrhagiae. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 17

### Leishmania major

In one example, the antigen is selected to be from Leishmania major. For detection of an antibody or antibodies specific to the antigen, 10 μl of serum, plasma, or whole blood of the test sample is first diluted with 150 μl of dilution buffer. The 150 of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 18

### Leptospira

In one example, the antigen is selected to be from Leptospira. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 19

### Listeria

In one example, the antigen is selected to be from a species of Listeria. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 20

### Moraxella

In one example, the antigen is selected to be from a species of Moraxella. For detection of an antibody or antibodies specific to the antigen, 10 μl of serum, plasma, or whole blood of the test sample is first diluted with 150 μl of dilution buffer. The 150 of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 21

### Mycobacteria

In one example, the antigen is selected to be from a species of Mycobacteria. For detection of an antibody or antibodies specific to the antigen, 10 of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 22

### Neisseria

In one example, the antigen is selected to be from a species of Neisseria. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 23

### Pasteurella

In one example, the antigen is selected to be from a species of Pasteurella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 24

### Pneumococcus

In one example, the antigen is selected to be from a species of Pneumococcus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 25

### Rickettsia

In one example, the antigen is selected to be from a species of Rickettsia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 26

### Salmonella

In one example, the antigen is selected to be from a species of Salmonella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 27

### Shigella

In one example, the antigen is selected to be from a species of Shigella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 28

### Staphylococcus

In one example, the antigen is selected to be from a species of Staphylococcus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 29

### Streptococcus

In one example, the antigen is selected to be from a species of Streptococcus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 30

### Treponema pallidum

In one example, the antigen is selected to be from Treponema pallidum. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 31

### Yersina

In one example, the antigen is selected to be from Yersina. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 λl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 32

### Candida albicans

In one example, the antigen is selected to be from Candida albicans. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 33

### Absidia

In one example, the antigen is selected to be from Absidia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 34

### Acremonium

In one example, the antigen is selected to be from Acremonium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 35

### Alternaria

In one example, the antigen is selected to be from Alternaria. For detection Of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 36

### Basidiobolus

In one example, the antigen is selected to be from Basidiobolus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 37

### Blastomyces

In one example, the antigen is selected to be from Blastomyces. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 38

### Coccidioides

In one example, the antigen is selected to be from a species of Coccidioides. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 39

### Cryptococcus

In one example, the antigen is selected to be from a species of Cryptococcus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 40

### Curvalaria

In one example, the antigen is selected to be from a species of Curvalaria. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 41

### Epidermophyton

In one example, the antigen is selected to be from a species of Epidermophyton. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 42

### Exophiala

In one example, the antigen is selected to be from a species of Exophiala. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 43

### Geotrichum

In one example, the antigen is selected to be from a species of Geotrichum. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 44

### Histoplasma capsulatum

In one example, the antigen is selected to be from a species of Histoplasma capsulatum. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 45

### Madurella

In one example, the antigen is selected to be from a species of Madurella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 47

### Malassezia

In one example, the antigen is selected to be from Malassezia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 48

### Microsporum

In one example, the antigen is selected to be from Microsporum. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 49

### Moniliella

In one example, the antigen is selected to be from Moniliella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 50

### Mortierella

In one example, the antigen is selected to be from Mortierella. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 51

### Mucor

In one example, the antigen is selected to be from Mucor. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 52

### Phialemonium

In one example, the antigen is selected to be from Phialemonium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 52

### Phialophora

In one example, the antigen is selected to be from Phialophora. For detection of an antibody or antibodies specific to the antigen, 10 of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 53

### Prototheca

In one example, the antigen is selected to be from Prototheca. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 54

### Pseudallescheria

In one example, the antigen is selected to be from Pseudallescheria. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 55

### Pseudomicrodochium

In one example, the antigen is selected to be from Pseudomicrodochium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 56

### Pythium

In one example, the antigen is selected to be from a species of Phythium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 57

### Rhinosporidium

In one example, the antigen is selected to be from a species of Rhinosporidium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 58

### Rhizopus

In one example, the antigen is selected to be from a species of Rhizopus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 59

### Scolecobasidium

In one example, the antigen is selected to be from a species of Scolecobasidium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 60

### Sporothrix

In one example, the antigen is selected to be from a species of Sporothrix. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 61

### Stemphylium

In one example, the antigen is selected to be from a species of Stemphylium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 62

### Trichophyton

In one example, the antigen is selected to be from a species of Trichophyton. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 63

### Trichosporon

In one example, the antigen is selected to be from a species of Trichosporon. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 64

### Xylohypha

In one example, the antigen is selected to be from a species of Xylohypha. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 65

### Babesia

In one example, the antigen is selected to be from a species of Babesia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 66

### Balantidium

In one example, the antigen is selected to be from a species of Balantidium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 67

### Balantidium

In one example, the antigen is selected to be from a species of Balantidium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 68

### Besnoitia

In one example, the antigen is selected to be from a species of Besnoitia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 69

### Cryptosporidium

In one example, the antigen is selected to be from a species of Cryptosporidium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 70

### Eimeria

In one example, the antigen is selected to be from a species of Eimeria. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 71

### Encephalitozoon

In one example, the antigen is selected to be from a species of Encephalitozoon. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 72

### Entamoeba

In one example, the antigen is selected to be from a species of Entamoeba.

For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 73

### Giardia

In one example, the antigen is selected to be from a species of Giardia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 74

### Hammondia

In one example, the antigen is selected to be from a species of Hammondia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 75

### Hepatozoon

In one example, the antigen is selected to be from a species of Hepatozoon. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 76

### Isospora

In one example, the antigen is selected to be from a species of Isospora. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 77

### Leishmania

In one example, the antigen is selected to be from a species of Leishmania. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 78

### Microsporidia

In one example, the antigen is selected to be from a species of Microsporidia. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 79

### Neospora

In one example, the antigen is selected to be from a species of Neospora. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 80

### Pentatrichomonas

In one example, the antigen is selected to be from a species of Pentatrichomonas. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 81

### Plasmodium

In one example, the antigen is selected to be from a species of Plasmodium. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 82

### Pneumocystis

In one example, the antigen is selected to be from a species of Pneumocystis. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 83

### Sarcocystis

In one example, the antigen is selected to be from a species of Sarcocystis. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 84

### Theileria

In one example, the antigen is selected to be from a species of Theileria. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 85

### Toxoplasma

In one example, the antigen is selected to be from a species of Toxoplasma. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 86

### Trypanosoma

In one example, the antigen is selected to be from a species of Trypanosoma. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 87

### Schistosoma

In one example, the antigen is selected to be from a species of Schistosoma. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 88

### Schistosoma

In one example, the antigen is selected to be from a species of Schistosoma. For detection of an antibody or antibodies specific to the antigen, 10 of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 89

### Adenovirus

In one example, the antigen is selected to be from a species of an adenovirus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 90

### Coronavirus

In one example, the antigen is selected to be from a species of a coronavirus. The coronavirus antigen may be a SARS antigen, for example. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once_the destaining buffer flushes the system, results may be read immediately. When both test position T and control position C appear red, a test result is positive for the presence of antibodies indicative of a particular disease, the antibodies specific for the antigen.

### Example 91

### Cytomegalovirus

In one example, the antigen is selected to be from a species of cytomegalovirus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 92

### Dengue Virus

In one example, the antigen is selected to be from a species of a Dengue virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 93

### Ebola Virus

In one example, the antigen is selected to be from a species of an Ebola virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 94

### Epstein-Barr Virus

In one example, the antigen is selected to be from a species of an Epstein-Barr virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 95

### Measles Virus

In one example, the antigen is from a species of a measle virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 97

### Chickenpox Virus

In one example, the antigen is from a species of a chickenpox virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 98

### Enterovirus

In one example, the antigen is selected to be from a species of an enterovirus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 99

### Hepatitis A

In one example, the antigen is a Hepatitis A antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 100

### Hepatitis B

In one example, the antigen is a Hepatitis B antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 101

### Hepatitis C

In one example, the antigen is a Hepatitis C antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 102

### Herpes Simplex

In one example, the antigen is a Herpes simplex virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

### Example 103

### HIV Virus

In one example, the antigen is a HIV 1 antigen such as p24 for detecting HIV-1. The p24 antigen also works for detecting a HIV-2 antigen. In this example, the p24 antigen consists of SEQ. ID. NO. 15, as follows:

PIVQNIQGQMVHQAISPRTLNAWVKVVEEKAFSPEVIPMFSALSEGAT
PQDLNTMLNTVGGHQAAMQMLKETINEEAAEWDRVHPVHAGPIAPG
QMREPRGSDIAGTTSTLQEQIGWMTNNPPIPVGEIYKRWIILGLNKIVR
MYSPTSILDIRQGPKEPFRDYVDRFYKTLRAEQASQEVKNWMTETLLVQN
ANPDCKTILKALGPAATLEEMMTACQGVGGPGHKARVL

For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen.

### Example 104

### HIV Virus

In one example, the HIV antigen is a HIV-1 gp 41 partial protein, which consists of SEQ. ID. NO. 16, as follows:

SELYKYKVVKIEPLGVAPTKAKRRVVQREKRAVGIGALFLGFLGAAGST
MGAASMTLTVQARQLLSGIVQQQNNLLRAIEAQQHLLQLTVWGIKQLQ
ARILAVERYLKDQQLLGIWGCSGKLICTTAVPWNASWSNKSLEQIWNN
MTWMEWDREINNYTSLIHSLIEESQNQQEKNEQELLELDKWASLWNWF
NITNWLWYIKLFIMIVGGLVGLRIVFAVLSVVNRVRQGYSPLSFQTHLPI
PRGPDRPEGIEEEGGERDRDR

For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen. Particularly good results were obtained by using this partial protein of gp 41.

### Example 105

### HIV Virus

In one example, the antigen used to detect HIV infection is a gp41 peptide fragment which consists of SEQ. ID. NO. 14, as follows:

QLQARILAVERYLKDQQLLGIWGCSGKLICTTAVPWNAS

For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies indicative of a particular disease, the antibodies specific for the antigen. Particularly good results were obtained by using this partial protein of gp 41.

Additional examples of antigen preparation may also occur before testing for the presence of an antigen. In one example of antigen preparation, an antigen preparation for a diagnostic kit comprises at least two HIV antigens, such as gp41 and p24 in a 1:1 ratio. For example, gp41 at a concentration of 1.6 mg/ml and a p24 concentration of 1.47 mg/ml may be prepared to a final concentration of 0.8 mg/ml gp41 and 0.735 mg/ml p24.

In another example of antigen preparation, an HIV-1 antigen, an HIV-2 antigen or both are used. In this example, an antigen mixture may be prepared. Peptide antigens gp41 and gp36 are dissolved in distilled H₂O at concentration of 2 mg/ml each. A p24 purified protein is suspended at a concentration of 2 mg/ml in 20 mM carbonate buffer (pH of 9.6). An antigen cocktail is prepared at ratio of gp41:gp36:p24 at a molar ratio of 5:2:3. The prepared antigen cocktail is then distributed into aliquots and kept in −20° C. degree. The antigen cocktail is immobilized on a filter paper made of a cellulose having a substantial α-cellulose content. In one example, the cellulose content is 98%, for example. A cellulose filter having a particle retention size of 20-25 μM and an ash percentage of 0.06% is used, for example. A frozen antigen cocktail may be thawed before loading to the cellulose filter paper. One microliter (μl) of antigen (about 2 μg) is loaded on the filter paper and is air-dried and stored at room temperature before assembling the antigen loaded filter paper in a test device.

In another example of antigen preparation, an antigen cocktail is prepared using peptide antigens gp41 and gp36 dissolved in distilled H₂O at concentration of 2 mg/ml each. A p24 purified protein is suspended at a concentration of 2 mg/ml in 20 mM carbonate buffer (pH of 9.6). For example, an antigen cocktail is prepared at ratio of gp41:gp36:p24 of 5:2:3. The prepared antigen cocktail may be distributed into aliquots and kept in −20° C. degree. Frozen antigen cocktail may be thawed before loading onto a cellulose filter paper. One μl of antigen (about 2 μg) is loaded on a cellulose filter paper, which is air-dried and stored at room temperature before assembling the loaded filter paper in a test device.

In one example, two HIV-1 antigens are used. The antigens are expressed in bacteria and purified using standard molecular biology methods. They may be a HIV-1 p24 protein, as previously discussed, and a HIV-1 gp41, which may be either be the whole protein, partial protein or peptide fragment.

In one example, a homologous sequence exhibits more than 80% identity with an amino acid sequence of a gp41 peptide, for example.

### Example 106

### Influenza Virus

In one example, the antigen is an influenza viral antigen such as an influenza A, B or C antigen, for example. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 107

### Leukemia Virus

In one example, the antigen is from a leukemia virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 108

### Marburg Virus

In one example, the antigen is from a Marburg virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 109

### Mumps Virus

In one example, the antigen is a Mumps viral antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 110

### Papilloma Virus

In one example, the antigen is a papilloma virus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 111

### Paramyxovirus

In one example, the antigen is a species of paramyxovirus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 112

### Pestivirus

In one example, the antigen is a species of pestivirus. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 113

### Picorna

In one example, the antigen is a picorna viral antigen. In one specific example of a picorna virus antigens, the antigens may include a poliovirus capsid antigen, or a pox virus antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 114

### Rabies Virus

In one example, the antigen is a rabies viral antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 115

### Reovirus

In one example, the antigen is a reovirus antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 116

### Respiratory Syncytial Virus

In one example, the antigen is a respiratory syncytial viral antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 117

### Rubella

In one example, the antigen is a rubella viral antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 118

### Rotavirus

In one example, the antigen is a rotavirus antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 119

### Vaccinia

In one example, the antigen is a vaccinia viral antigen. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 120

### Human T-Lymphotropic Virus

In one example, the antigen is a human T-lymphotropic viral antigen. For detection of an antibody or antibodies specific to the antigen, 10 μl of serum, plasma; or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 121

### Prostate Cancer

In one example, the antigen is from a non-infectious disease, such as cancer. In a specific example of a cancer, the cancer is prostate cancer and the antigen is a prostate specific antigen (PSA). For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 122

### Alzheimer's Disease

In one example, the antigen is an A68 antigen, from Alzheimer's disease. For detection of an antibody or antibodies specific to the antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

### Example 123

### Combination of Viral and Bacterial Antigens

In one example, two or more antigens may be detected by the test kit. The two different antigens may be a viral and a bacterial antigen, for example. The bacterial antigen may be a Mycobacterium Tubercolis. The viral antigen may be a Hepatitis antigen or a HIV antigen, for example. For detection of each antigen, 10 μl of serum, plasma, or whole blood of the test sample is first diluted with 150 μl of dilution buffer. The 150 μl of the now diluted sample is then added to the center of the test device. For a blood sample, it is advised to wait for about three minutes or until a diluted sample is a clear red, before going on to the next step of loading the diluted sample.

Once the diluted sample is absorbed, 150 μl of a staining buffer is added. In one example, the staining buffer is Protein A coupled to colloidal gold. Once the staining buffer is absorbed, 200 μl of destaining buffer is added. The destaining buffer may be Dulbecco's Phosphate Buffer Saline (1×) (DPBS) solution, for example. Once the destaining buffer flushes the system, results may be read immediately. When both test position T and control position C appear red, a test result is positive for the presence of antibodies in a particular disease, the antibodies specific for the antigen.

Combinations that are tested using the test kit are not merely a combination of viral and bacterial antigens. In another example, a combination of two or more viral antigens may be tested. In another example, a combination of viral, parasital, bacterial and fungal antigens may be selected. In one example of type of combination of viral, parasital, bacterial and fungal antigen, a combination of fungal and viral infections may be tested. The combinations described herein are not limited to the specific examples disclosed.

In one example, a plurality of two or more test dots will be present, indicating that the test result is positive for the presence of antibodies for two or more particular diseases. Thus, two test dots testing for a combination of viral and bacterial antigens indicate that the person has antibodies for a viral disease and a bacterial disease.

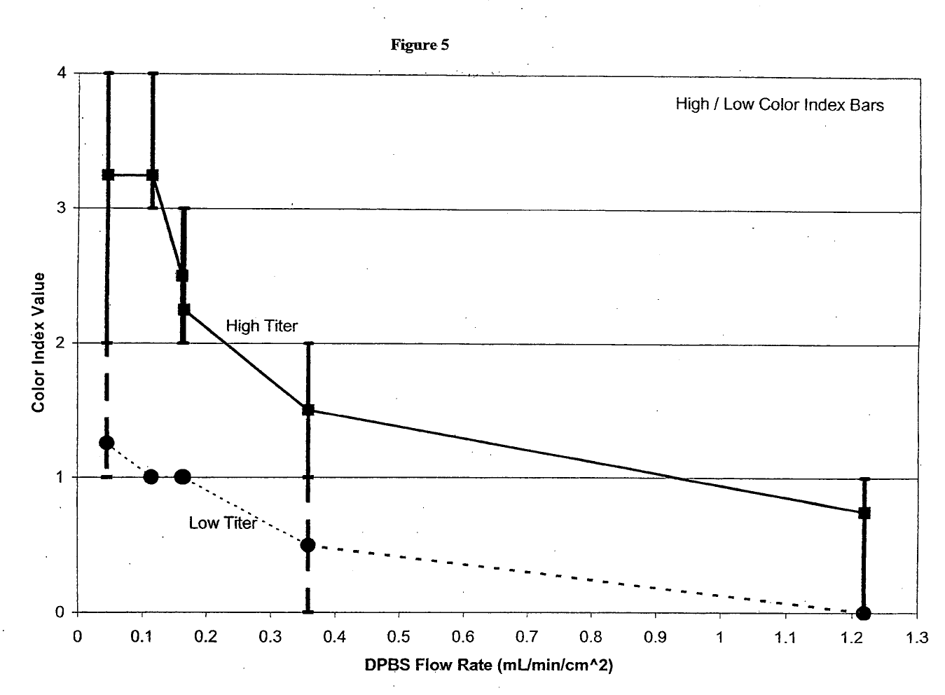

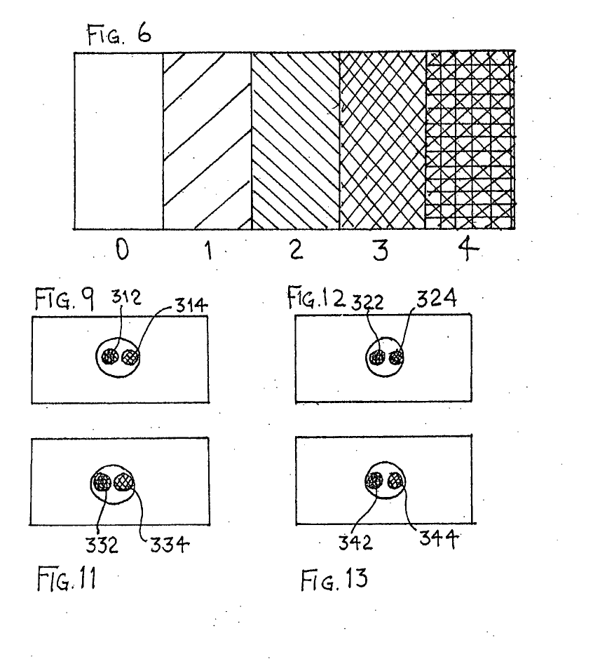

In the artists sketch of FIGS. 3A and 3B, results of a sample using a glass fiber membrane 160 are compared to a rapid test kit using one or more HIV antigens for detecting the presence of HIV in a sample of plasma. The glass fiber membrane 160 of FIG. 3A failed because plasma could not flow through. In comparison, the sample illustrated in FIG. 3B, using a cellulose filter paper 180 and otherwise similar in physical characteristics, successfully indicated a test positive for HIV with much better contrast than prior art test kits. The control spot 172 is apparent, and positive test spot 174 matches the color index value of the control spot 172. The characteristics of glass fiber membranes utilized for this test are described in Table 9. In FIGS. 4A-C, an illustration of a plasma sample tested using a nitrocellulose membrane 200 is compared with a sample tested using a cellulose filter paper 220. The nitrocellulose membrane as presented in the sketch of FIG. 4A fails, providing poor contrast and requiring a longer time to perform the test than for the rapid test kit of FIG. 4C, using cellulose reaction layer 220. A red residue of colloidal gold solution remains in the testing region of FIG. 4A that did not pass through the nitrocellulose reaction membrane. The nitrocellulose membrane 200, obtained from Bio-Rad Laboratories, had a pore size of 0.45 microns. Using a cellulose reaction layer 220, as illustrated in FIG. 4C, the control spot 182 is clearly evident, as is a positive test spot 184, which matches the color index value of the control spot. In FIG. 4B, a device membrane 205 uses a nitrocellulose mixed ester membrane having a pore size of 5 microns. Plasma fails to flow through, causing this membrane to fail also.

In FIG. 5, a flow rate of PBS is measured using a modified ASTM Standard for measuring flow rate through a 7 cm circle of filter paper folded in quarters and suspended in a ring. Then, several filter papers were used to make test kits using the same antigens and loading. Tests were performed using HIV positive samples, and the color intensity of test spots were determiend using the color index value chart of FIG. 6. FIG. 5 shows a graph of color index value versus DPBS flow rate. Measurements are shown for water and phosphate buffered saline, using qualitative filter paper and wet strengthened filter paper having various ratings for particle retention size. Data for FIG. 5 is found in Table 2. The error bars in FIG. 5 represent high and low data values. High titer samples are shown with squares and low titer with circles.

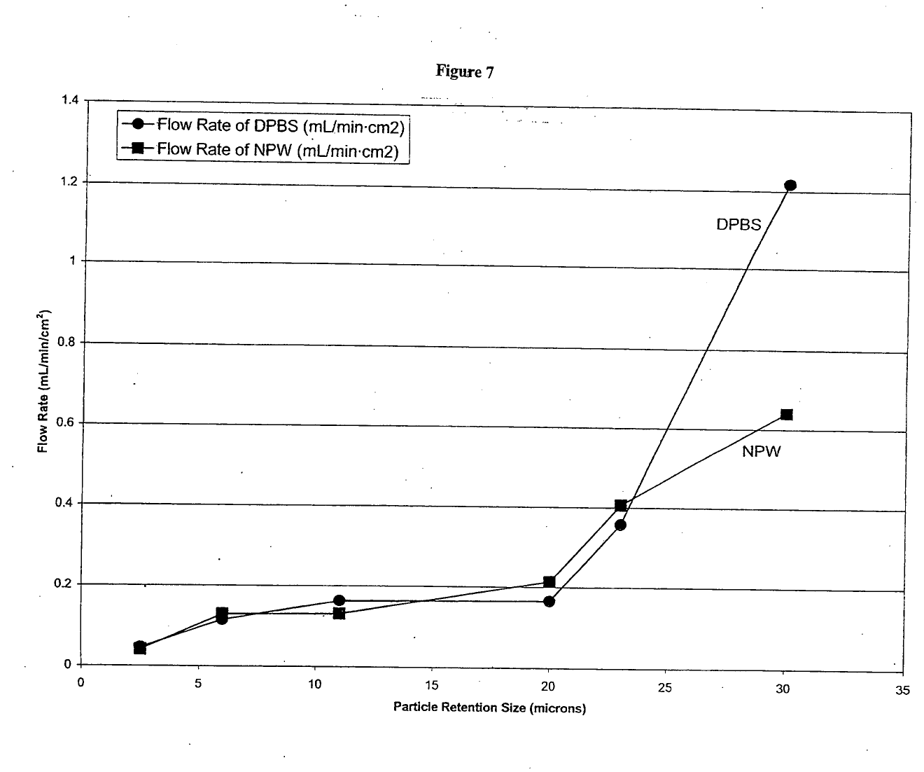

Flow rate is correlated with color index values. The low flow rates are more sensitive than higher flow rate membranes. In this example, six different types of Whatman™ filter paper were tested; each having particle retention sizes ranging from 2.5 microns to 30 microns. Surprisingly, as shown in FIG. 7, the flow rate showed a plateau region between about 6 to 20 microns with a flow rate of about 0.1 to 0.2 mL/min/cm². The plateau region corresponded to an optimal combination of sensitivity and flow rate for testing samples, whether based on blood, serum or plasma, in one example of an HIV antibody sensitive test kit.

A modified ASTM Standard measurement was used to determine the flow rate of each membrane. Filter paper was dimensioned to a 7 cm diameter circle. The paper was placed in filtering solution (both PBS and water were tested) for a time sufficient, for the paper to be completely soaked. Then the paper was placed flat in a funnel, except for edges, which were folded upwards. Then, 5 ml of filtering solution was added to the center of the funnel and time was measured using a stopwatch. When an amount of the filtering solution had passed through the filter, the time was recorded.

The flow rate, in units of mL/min/cm² is calculated in the following manner: V/T×60 s/1 min×1/cm², where V=volume in ml, T=time in seconds, s=seconds, min=minute, and surface area expressed as cm².

A filter paper of qualitative type had pore sizes of 2.5, 6, 11 and 20-25 microns (which we have graphed as 20 microns). A wet strengthened filter paper had a particle retention size of 23 and 30 microns. The flow rates in water for a filter paper with a particle retention range from 2.5 microns to 23 microns is in the range of about 0.04 mL/min/cm² to about 0.4 mL/min/cm²′ The flow rates in DPBS are also in the range of about 0.04 mL/min/cm² to 0.4 mL/min/cm². It is not clear that there is any statistical significances in the measured differences between water and PBS. The term “about”, as it is used with flow rates, takes into consideration experimental errors introduced in any measurement as is known to a person of ordinary skill in the art. The flow rates for nitrocellulose mixed ester membranes are much higher than those measured for cellulose filter paper, as measured in units of mL/min/cm² and shown in Table 1B, below.

For example, the contrast between data measured in Table 1A and reported in Table 1B are striking when comparing flow rates in water for a wet strengthened cellulose filter paper of 23 microns with a nitrocellulose mixed ester membrane with a pore size of 5 microns. The nitrocellulose mixed ester membrane had a flow rate several orders of magnitude greater. Previously, as illustrated by FIG. 4B, a sample using a nitrocellulose mixed ester membrane having a pore size of 5 microns failed. In addition, the flow rates for a paper-backed nitrocellulose having a pore size of 0.45 microns resulted in a much faster flow rate of 6 ml/min/cm², as reported by Chan et al., in paragraph [0171] of U.S Patent Publication No. 2004/0002063.

The nitrocellulose mixed ester membrane filters used were Magna™ Nitrocellulose mixed ester membrane filters, manufactured by GE Infrastructure Water and Process Technology. The pore size used in the example is 5 microns. The flow rate of nitrocellulose mixed ester membrane in water was measured in mL/min/cm² measured at 520 mmHg (10 psi), at 20 degrees Celsius. The air flow rate is measured in units of L/min/cm², of filtration area, measured at 520 mmHg (10 psi), at 20 degrees Celsius (68 degrees Fahrenheit). The Bubble Point pressure occurs at which air is first forced through pores of water-wet membrane.

Properties of cellulose filter papers is shown in Table 1C. Table 1C, obtained from a Whatman web site shows typical properties of cellulose filters tested, such as particle retention liquid, and airflow rate. Such properties may be used to select for a particular filter paper. Grades 1, 3, 4, 5, 113 and 114, as reported in Table 1C, were utilized in preparing test kits. Wet strengthened qualitative cellulose filters contain a small quantity of a chemically stable resin to give improved wet strength. For these tests, filter paper is cut down into circles with a diameter of 7 cm for flow rate measurements, and the filter papers were dimensioned to 1 inch by 1 inch squares for use in test kits.

Table 2 shows the respective color index values for each low titer and high titer sample tested at a respective particle retention size and flow rate. FIG. 5 shows mean color index and the color index bars show high and low values of the color index. Samples having a color index value of 1 are considered to be low titer samples, while samples having a color index value greater than 2 considered to be high titer samples. The cellulose filter paper with about a 1.2 mL/min/cm² flow rate failed on each low titer sample. At a flow rate of about 0.4 mL/min/cm², a rapid test kit successfully detected a high titer, HIV-positive sample, but the same test kit failed two times in four tests to detect the presence of a low titer, HIV-positive sample. Thus, for cellulose reaction layers, of flow rate of about 0.4 mL/min/cm² is an upper limit for application as an HIV screening test. Surprisingly, by adopting a color index scale for quantifying intensity of results, an example of a rapid test kit exhibited sufficient sensitivity and specificity to be used to distinguish between low and high titer HIV-positive samples.

The data in FIG. 5 show that the color index values roughly fall off exponentially with the flow rate of cellulose filter paper. Thus, it is believed that even higher flow rates would result in more failures. Table 2 describes the data for low and high titer samples. DPBS flow rates, particle retention sizes and results for test and control samples are shown.

FIG. 6 illustrates, schematically in a black and white line drawing, a color index for semi-quantitative determination of the sensitivity by measuring color index values. The background associated with 0 indicates that no contrast is visible between a test spot and the background. Anything darker than background is a 1, which is represented by light shading in FIG. 6. A value of 1 or greater is deemed a positive test result. A color index value greater than 2 corresponds to the high titer samples and is represented in FIG. 6 by darker shading. A higher contrast between background and the test spot is represented by cross hatching 3, and the highest contrast is represented by double cross hatching 4. This schematic representation relates to actual colors that are shown in the disclosure of U.S. patent application Ser. No. 12/008,861, which is incorporated by reference.

In FIG. 6 is an example of a color index chart, the scale runs from 0, which is negative for the presence of an antibody or antibodies specific for a given antigen, to a 4, which is the highest semi-quantitative value. An index value of 0 indicates that pink staining of the background may occur but does not indicate presence of a discernable dot. An index value of 1 is distinguishable from the background, but is not darker than the color represented by 1. An index value of 2 indicates a clearly visible dot darker than 1, but not darker than 2. A value of 3 is a highly intense dot darker than 2 but not darker than the reference provided at 3. A color index value of 4 is darker than the reference labelled 3. For example, comparison of plasma and blood samples obtained from the same donor sample are shown in FIG. 9, for example. Blood tests (a), (b), have control spots 312, 332 and test spots 314, 334 comparable in color index value to the control spots 322, 342 and test spots 324, 344 of plasma samples (c), (d). Both whole blood and plasma may use the same test kit with the same color index value chart.

Unless specified otherwise, comparisons with other test kits are made between commercial kits and examples using a cellulose filter paper having a flow rate of about 0.1 mL/min/cm². In FIG. 7, a graph of flow rate versus particle retention size is presented for the data shown in Table 2 previously. Increased pore size shows increased flow rate, but increased flow rate, has decreased assay sensitivity, as shown in FIG. 5. Preferably, the sensitivity yields results capable of distinguishing high and low titer, while also providing as rapid a test as possible.

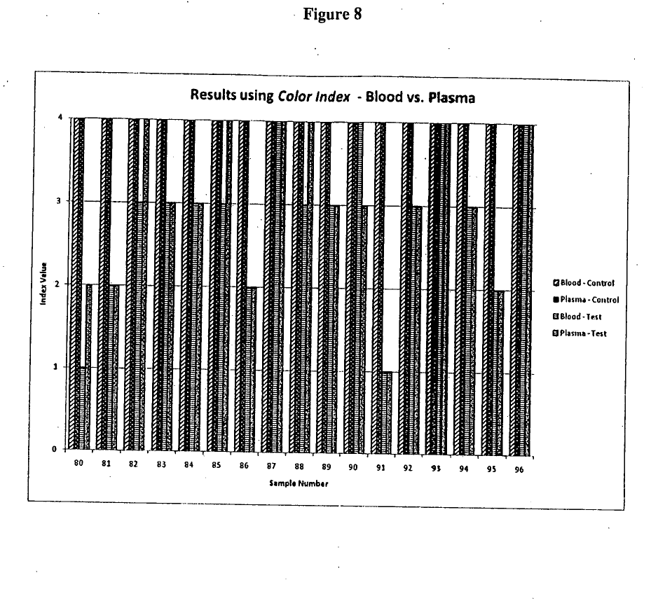

FIG. 8 shows a comparison of tests using samples of blood and plasma. The color index values are measured. The results for blood and plasma tests are remarkably similar which is very surprising and unexpected. Most tests kits cannot be used to test whole blood. As can be seen with other types of reaction membranes tested in the figures, all of the others are inoperative when used with the blood rather than plasma or serum. Use of whole blood allows testing to be conducted in the field where centrifuges are not easily available, and represents a substantial advantage over other test kits.

Table 3 shows data reported graphically in FIG. 8. Some samples were tested twice, while other samples were tested once. Table 3 compares data for samples using blood and samples using plasma, from the same source and using the same type of celulose reaction layer.

Plasma, serum and blood samples all have similar visual results. For example, a comparison of plasma and blood samples is shown in FIGS. 9, 11, 12 and 13. Examples using whole blood (FIGS. 9, 11) and blood plasma (FIGS. 12, 13) are schematically represented and tested positive for HIV. These images are represented by test sample 84, as reported in the tabulated data of Table 3. Positive tests spots for presence of HIV are indicated by test spots 314, 324, 334, 344 and control spots 312, 322, 332, 342. Test samples 260 and 262 were obtained from the same donor sample. One used blood while the other used plasma. Similarly, test samples 270 and 272 were obtained from the same donor sample, with one for blood and one for plasma. Both blood and plasma samples tested 3 on the color index scale.

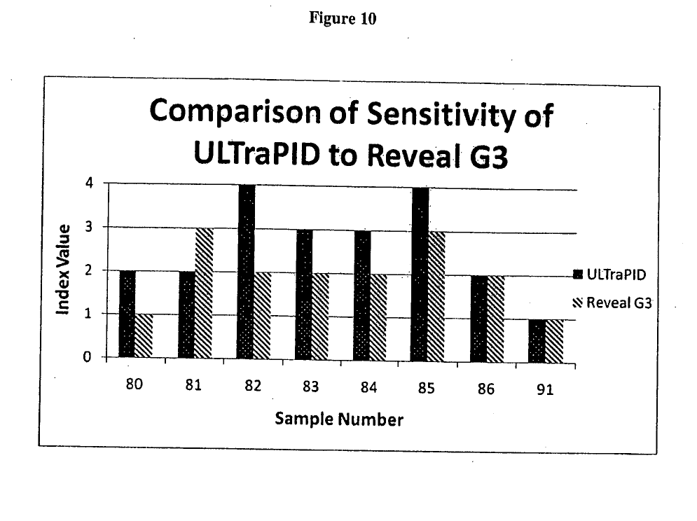

FIG. 10 shows a bar graph representing color index values for various samples using a rapid test kit with a flow rate of about 0.1 mL/min/cm² in DPBS and a commercial assay, using the Reveal® G3. Most of the plasma samples using the test kit had better contrast than plasma samples using MedMira® Reveal® G3 test kit.² In FIG. 11, some representative comparisons of a rapid test kit with a Reveal® G3 kit are shown. Rapid test kits having cellulose filter paper with a flow rate of about 0.1 mL/min/cm² in DPBS were tested. The procedure for using a rapid test kit includes adding 150 microliters of Phosphate Buffer Saline (PBS) solution is added to a freeze dried staining buffer. 10 microliters of plasma are diluted with 150 microliters of PBS solution. The kit is loaded with the diluted plasma, 150 microliters of staining buffer, and 200 microliters of PBS solution in succession. The test duration is less than two minutes, qualifying as a rapid test. For a rapid test kit, blood and serum, alternatively, may be used, in addition to plasma. This is not the case for other commercial test kits. ²MedMira® and Reveal® are registered trademarks of MedMira Laboratories, Inc., Toronto, Canada.

The Reveal® G3 kit used 3 drops of Universal Buffer added to the kit, followed by 1 drop of plasma. Then 3 drops of Universal Buffer are added to the kit. Then an instant gold cap was added on the kit, with 12 drops of Universal Buffer added through the cap. Optionally, an additional 3 drops of Universal Buffer may be added. The test duration is less than three minutes. The term “Universal Buffer” is used in the instructions for the Reveal® G3 kit. Test kit 400 tested HIV positive, which is the same result as the test kit 300 of Reveal® G3. Both kits tested 1 on the color index scale. The Reveal® G3 of FIG. 11(f) shows a G3 test kit 320 that tested patient sample BBI #10 as negative. Rapid test kit 420 for sample BBI #10 tested positive, having a color index of 1. Thus, the rapid test kit 420 indicated HIV-positive even though the Western blot showed indeterminate. Like BBI # 4 from test kit 440, and BBI #25 from sample 460, sample BBI #10, was from an Anti-HIV-1 PRB204 performance panel purchased from BBI Diagnostics, which had tested the panel on different kits. A comparison of BBI #10 with other competing kits showed that sample BBI #10 is positive using an Abbott Determine™ HIV-1/2. Other kits such as OraQuick® and Uni-Gole tested negative.³ A Western blot test of BBI #10 was indeterminate. BBI refers to screening assay PRB 204, which is shown in Tables 4 and 5. While Western blot is the gold standard, an indeterminate Western blot fails to identify either a positive or negative test result for HIV. ³ OraQuick® is a registered trademark of Orasure Technologies; Uni-Gold™ is a trademark of Trinity Biotech.

Test kit 440 containing sample BBI #4 tested HIV positive like test kit 340 using Reveal® G3. Rapid kit 440 tested 3 on the color index scale using cellulose reaction layer, while Reveal® kit 340 tested 1 on the same color index scale, showing better contrast for the cellulose test kit 440, making the test kit 440 easier to read. Other kits such as OraQuick® and Uni-Gold™ also tested positive. A Western blot panel data also resulted in a positive result. Accordingly, for positive test results, the rapid test kit example using cellulose filter paper correctly identified a test result positive for HIV

A Reveal® G3 kit 360 using sample BBI #25 tested negative. On the color index scale used by us, the kit 360 tested 1 on a color index scale. Test kit 460 tested negative and 0 on a color index scale. A third kit, one from Abbott, for the same sample BBI #25 showed a positive result. Both OraQuick® and Uni-Golf™ tested positive. The Western blot test was indeterminate. However, the test kit, unlike Reveal® G3, allows use of blood in addition to serum or plasma. Serum and plasma samples, require laboratory equipment and more time to prepare the samples than blood. The membrane used in the test kit also absorbs quickly. From a color index measurement, this test determined that the test kit sample 460 was low titer with a color index value of 1. Thus, a rapid test kit may be used for screening, and using a color index scale, may also serve as a qualitative assay of antibody titer.

Further results for all the BBI samples tested, and a comparison of the test kit with Western blot results and competing test kits are shown in Tables 4-6. For example, BBI #1 corresponds to PRB 204-01, BBI #2 corresponds to PRB 204-02, and so forth. Tables 7 and 8 report the band patterns for the Western blot tests from the BBI panel. The Western blot band patterns are shown in Table 5 for each of the samples in the assay.

The Reveal® G3 (a) kit has a nitrocellulose membrane; therefore, a test with blood using the Reveal® G3 kit failed, while a rapid test kit successfully found the sample to be negative for HIV. Blood did not flow through, but instead coagulates, in a Reveal® G3 test kit. The rapid test kit that successfully tested the blood contained filter paper with a flow rate of about 0.1 mL/min/cm² in DPBS. In addition, a glass fiber membrane was tested. Blood did not flow through the glass fiber membrane but instead coagulated on the surface. The glass fiber membrane that was used was a Whatman® GF/C. While Chan, in U.S Patent Publication No. 2004/0002063, taught the use of glass fiber membranes for use with blood samples, these tests clearly showed that using glass fiber membranes in test kits failed for tests using whole blood, without using the complex procedures of Chen. The Reveal® G3 test kit also cannot utilize blood samples, completely failing in that regard. Like the Reveal® G3 test kit, the test kit of Mahajan in U.S. Patent Publication No. 2004/0023210, ultilizes a nitrocellulose membrane, and also limits its use to serum and plasma. Thus, a rapid test kit is capable of better contrast using blood, plasma and serum, which is a significant and important improvement for a rapid test kit.

Nitrocellulose is well-known in the art for binding proteins, which is why it is routinely used in Western blots and other assays. However, none of these nitrocellulose assays use cellulose reaction membranes, and none are suitable as a rapid assay for use with whole blood. Alternatives to nitrocellulose are seldom considered for use in test kits. In the tests conducted with blood it is clear that nitrocellulose fails, while cellulose selected in an operative flow rate range, such as 0.04-0.4 mL/min/cm² works as well with blood as with plasma and serum. The added flexibility makes the test suitable for use as a field test. Surprisingly, there is no loss of sensitivity or specificity with the use of blood in some example test kits used for testing HIV-positive samples.

Table 6 shows characteristics of a glass fiber membrane and shows data for glass fiber membranes. For particle retention, the following is assumed: 2% initial penetration values using solid particulates dispersed in water. (Represents complete retention in normal laboratory analysis.). For flow rate, the following is assumed: Vacuum filtration of prefiltered water through 2 1/16 in. (5.5 cm) flat filter at 100 mmHg (1.9 psi). Water absorbance assumes that there is an equilibrium volume of water absorbed by filter.

In additional tests, example rapid kits are compared to Reveal® G3 kits using high and low titer samples of blood plasma. All test kits shown in the examples use a cellulose filter paper selected with a PBS flow rate of about 0.1 mL/min/cm², unless otherwise specified herein. Example test kits 500, 502, 504, 506, 508, 510 used specimens #80, #81, #82, #83, #84, and #91, respectively, and had better visual contrast than corresponding Reveal® kits 488, 490, 492, 494, 496 and 498. Color index values for samples are represented in Table 7. The data show that all the test kits, except for one, sample #81, had better visual contrast than Reveal® G3 kits for the same plasma samples tested.

### Genetic Probe Examples

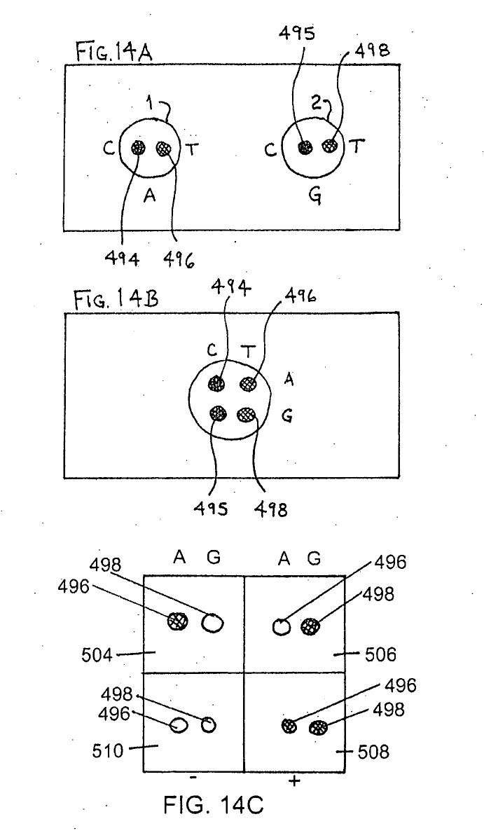

FIGS. 14A-C illustrate, schematically, examples of a test kit having both an antibody-based test spot 496 and a genetic probe test spot 498, in addition to the control test spots 494, 495, for example. In FIG. 14A a single test kit has two testing windows 1, 2, which contain test regions for an antibody test 1 and a genetic probe 2, respectively. In FIG. 14B a single test kit has both an antibody test region 496 and a genetic probe region 498 in a single test window. The kit in FIG. 14B may have a procedure that uses a single staining step or may have a sequence including a staining agent for the antibody test separate from application of the staining agent for the genetic probe. It is preferred for a point of care test to keep the number of steps and the complexity of steps to a minimum; therefore, it is preferred to combine the two staining agents, antibody and genetic probe, and to add them in a single step to a single window, such as illustrated in FIG. 14B, for example. For example, a staining buffer may have a viral-specific genetic probe coupled to a nanotube or a particle, such as a colloidal gold particle, gold nanoparticle, silver nanoparticle, carbon nanotube or the like. FIG. 14C graphically illustates four possible outcomes of a test kit combining both antibody and genetic probe test regions 496, 498, when it is assumed that the control spot is properly demonstrated. A positive antibody spot 496 and a negative genetic probe spot 498 produces a first result 504 indicating the presence of antibodies but having no indication of the virus. This HIV-negative indication would suggest innoculation or the presence of maternal antibodies. A second negative result 510 could be negative for both antibodies and RNA. Two possible positive results 506, 508 might be demonstrated with either a positive indication for the genetic probe test region 498. The presence or absence of antibodies may be a significant indication, leading to a different course of treatment or clinical monitoring regime, for example. In one example, a patient might indicate positive for the presence of the virus prior to indicating positive for the presence of antibodies to the virus, due to a delay in being able to detect the presence of antibodies in the blood, for example.

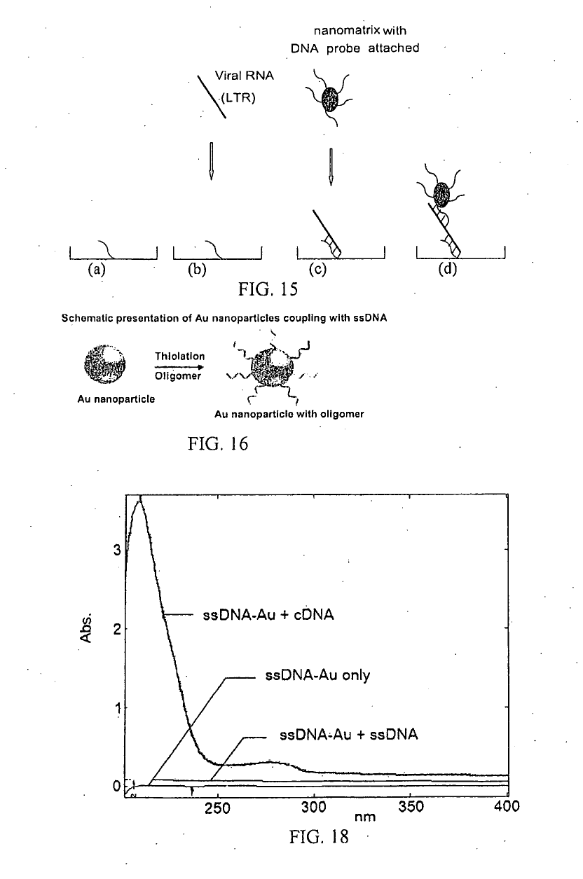

A probe for an RNA or DNA compatible sequence may be immobilized onto blotting paper or filter paper, which may be cellulose filter paper or nitrocellulose filter paper, for immobilizing RNA or DNA having the compatible sequence, as illustrated in FIG. 15 (a), for example. Preferably, cellulose filter paper is used for detecting the presence of a specific RNA, DNA or fragment thereof in a volume of bodily fluid passing through the filter paper, which allows for the passing of a significant volume through the surface of the filter paper in a short period. For example, a primer or a pair of primers may be used as a genetic probe with one primer being attached to a marker, such as a gold nanoparticle, and being included in the staining buffer, and another primer being immobilized on the paper in a spot or other indicator region of the paper to capture a specific RNA or DNA sequence. Each of the pair of primers are complementary to a specific RNA or DNA sequence, such as a viral RNA or single stranded DNA, for example.

By passing a bodily fluid containing the specific RNA or DNA sequence through the paper, the specific RNA or DNA is captured by the primer immobilized on the paper, as illustrated in FIGS. 15 (b) and (c), for example. Then, by passing the staining buffer through the same paper, the primer attached to a gold nanoparticle, or other particles or nanotubes, is immobilized on the specific RNA or DNA that is immobilized on the paper, as illustrated in FIG. 15(d), providing a visual contrast compared to a portion of the paper having no immobilized primer. Contrast may be enhanced by a chemical reaction, such as in photodevelopment, fluoroescence, such as under an ultraviolet light, or electrical properties, such as conductance, resistance or the luck. In one example, the marker, which may be a nanoparticle or nanotube, fluoresces or phosphoresces, such as when exposed to ultraviolet light, for example. A rinsing solution may be used to wash residual staining buffer from the paper to provide enhanced contrast between the spot and the background, because the rinsing solution removes staining buffer only from the background and not the immobilized RNA or DNA complexes captured on the test spot. Then, the contrast between the spot and the background may be analyzed to determine the presence of a sequence of RNA or DNA in the bodily fluid, and in some examples, a relative level or concentration of the sequence of RNA or DNA in the bodily fluid may be determined, either qualitatively or quantitatively. For example, the resulting contrast on the test kit may be compared to a plurality of known viral loads, such as concentrations of 5000, 10,000 and 15,000 viral copies per milliliter. The plurality of known viral loads may be used to provide a standard intensity of a marker region or of a contrast between a marker region and a background of the test kit. By comparing a test kit to the standard, the comparative concentration range of the viral load may be determined qualitatively or quantitatively.

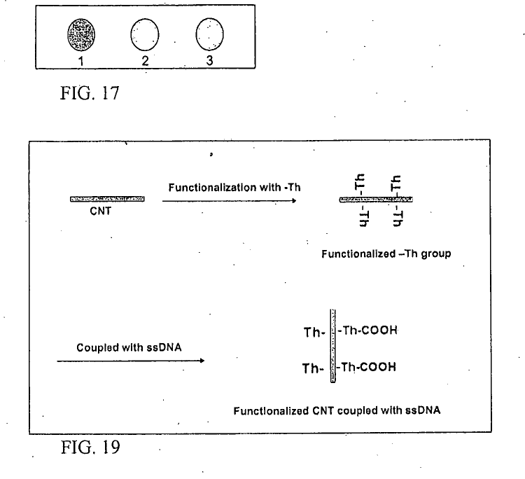

FIG. 16 schematically illustrates a gold nanoparticle before and after thiolization and functionalization with an oligomer, such as a single strand oligonucleotide. In FIG. 17, an LTR oligonucleotide is used as an example for detecting viral load using a complementary pair of primers. Primers may be derived from the LTR region of HIV-1 isolates, for example. Contrast is visible between the spot (1) and the background under ultraviolet light, because there is an interaction with ssDNA-Au-RNA and complementary DNA (cDNA) to immobilize the nanoparticles, which are gold in this example, to a spot on the filter paper, as illustrated schematically in FIG. 15. However spot (2) results from an interaction only between ssDNA-Au and ssDNA, and spot (3) results from ssDNA only, without genetic probe interactions. An absorbance (abs.) spectrum is shown in FIG. 18 that differentiates a sample of sDNA-gold nanoparticels -RNA with complementary DNA from both ssDNA-gold nanoparticles alone and ssDNA-gold nanoparticles with ssDNA, as a control. Contrast is good for a a sample of blood having HIV-1 present. Thus, the genetic probe is capable for use in detecting the presence of HIV using the complementary DNA as a target for binding a marker to the marker region of a test kit.

An example of a test using a flow-through rapid test kit detects viral RNA using an LTR-specific DNA genetic probe attached to gold nanoparticles. For example, preparation of DNA-gold nanoparticle complexes may proceed as described by Mirkin et al. using citrate-stabilized gold nanoparticles and thiol modified DNA oligomers. In one example, 5′-fluorescein, 3′-thiol labeled oligonucleotides may be used for determining surface coverages. A bifunctional DNA-gold nanoparticle conjugate may be prepared by adding a mixture containing the desired amount of oligonucleotides to an aqueous nanoparticle solution as reported by J. J. Storhoff, R. Elghanian, R. C. Mucic, C. A. Mirkin, R. L. Letsinger in the J. Am. Chem. Soc. (1998) vol. 120, pp. 1959-1964, for example.

Functionlized gold nanoparticles conjugated with a single strand oligonucleotide may be mixed with 0.5% of a 10% bovine serum albumin in phosphate buffer (BSA) solution (e.g. pH 7.4, BD), which may be dropped on a glass plate for spectral analysis using a ultraviolet spectrophotometer, for example. The ultraviolet spectrophotometer results in FIG. 18 are capable of distinguishing the plate with the functionlized gold nanoparticles conjugated with single strand oligonucleotide, without or without additional single stranded oligonucleotide, from a similar plate with a complementray oligonucleotide added by dropping 25 microliters of the complementary oligonucleotide on the functionalized gold nanoparticles conjugated with single strand oligonucleotide.

In one example, an HIV genetic probe is prepared using functionalized gold nanoparticles conjugated with a single strand oligonucleotide for detecing the presence of HIV. A single stranded DNA primer with a functionally identical sequence of HIV-1 LTR is synthesized and immobilized in a spot on the cellulose filter paper of a rapid test kit of the present invention. A bodily fluid, such as raw blood, blood plasma, urine, saliva, or the like, is added to a buffer solution or placed directly on the filter paper of a rapid test kit for a flow—through test. The HIV RNA (e.g. HIV-1 LTR) hybridizes with the DNA primer on the filter paper. Then, a staining buffer including the functionlized gold nanoparticles conjugated with a DNA probe is added to the filter paper, such that the functionalized gold nanoparticles conjugated with the DNA probe hybridize with the HIV RNA. The gold nanoparticles concentrate at the HIV RNA, providing a red-tinted contrast to the background. If necessary, a destaining buffer may be used to further rinse the staining buffer from the background, while having no effect on the gold nanoparticle complexes hybridized to the HIV RNA. In one example, a plurality of different DNA probes are selected to be complementary to a plurality of different regions of HIV RNA, such that one HIV RNA molecule has the possibility of binding several gold nanoparticles, improving contrast and senstivity of the rapid test kit for each type of HIV RNA detected by the test kit. An important advantage of using a DNA probe is that it is the HIV RNA, itself, that is detected; therefore, vaccinated individuals will be negative in a test using a DNA probe and the level or concentration of HIV in a volume of bodily fluid may be compared and analyzed, directly. A test kit detecting only antibodies for HIV could indicate a positive test for a vaccinated individual and could only be used to show the level of antibodies produced by the individual, not the level of the virus, itself. A rapid test kit testing for both antibodies in one spot and using a DNA probe for another spot may provide both detection of antibodies and either a qualitative or quantitative analysis of an HIV viral load in the bodily fluids of an individual.

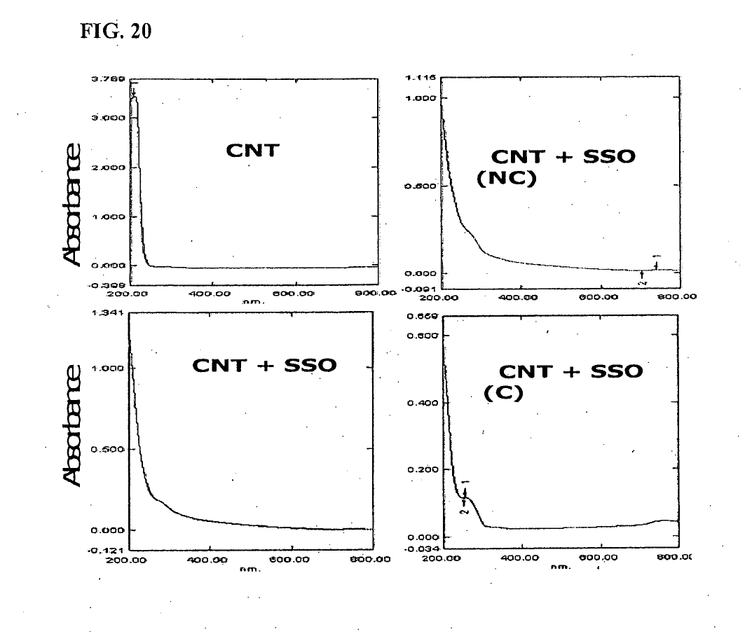

FIG. 19 illustrates schematically an example of coupling carbon nanotubes with an oligonucleotide. In this example, carbon nanotubes, such as single walled carbon nanotubes or herringbone carbon nanotubes, are dispersed in a dimethylfromamide under ultrasonic agitation. Two hours of agistation is adequate for a sample of 1 milligram of carbon nanotubes in 2 millileters of dimethylfomamide, for example. This provides a carbon nanotube suspension with a density of 5 milligrams per milliliter having a black color. Then, the carbon nanotubes in suspension are thiolated by thoroughly mixing 2 milliliters of a 1 mM solution of a thiol having a thiol group (positively charged), such as a mercaptan, into the carbon nanotube (negatively charged) suspension. Centrifugation at 18,000 rpm for 15 minutes is adequate to collect the thiol-functionalized (or thiolated) carbon nanotubes by removing the supernatant liquid. Distilled water may be used to wash the thiolated carbon nanotubes, at least three times in one example, to rinse away any of the unbound thiol molecules. A mass of 10 milligrams of single stranded DNA may be added to 1 milligram of thiolated carbon nanotubes in 1 millileter of PBS (pH 7.0) at 4 degrees centigrade for twelve hours, for example. Afterwards, the suspension is centrifuged and the ssDNA-sulfur-carbon nanotube complexes are collected by removing the supernatant. Again, the DNA functionlized carbon nanotubes may be rinsed thoroughly to remove all of the unbound molecules of ssDNA. As illustrated in FIG. 20, carbon nanotubes alone (A) have a different ultraviolet absorbance spectrum from carbon nantoubes conjugated with single strand oligonucleotides (B), which have a different absorbance spectrum compared to (C) non-complementary oligonucleotide (DNA) fragments and (D) complementary fragments of single strand oligonucliotides hybridized with carbon nanotubes conjugated with single strand oligonucleotides. Thus, a genetic probe using carbon nanotubes conjugated with ssDNA may be used to detect the presence of complementary DNA, qualitatively or quantitatively, for example.

In an example of a method of assay, single strand DNA (ssDNA) functionlized carbon nanotubes may be included in a staining buffer that is added to a rapid test kit of the present invention after a bodily fluid, with or without dilution in a buffer solution, is deposited on the filter paper of the test kit. The filter paper is prepared by including a spot with an immobilized genetic probe on the spot that is capable of immobilizing viral RNA or the DNA to be detected. Thus, the hybridizing of viral RNA or the complementary DNA with the ssDNA functionlized carbon nanotube binds the ssDNA functionalized carbon nanotube preferentially at the test spot of the rapid test kit, providing a contrast between the test spot and the background, for example.

The spectra in FIG. 20 were obtained by preparing a glass slide with ssDNA functionalized carbon nanotube dropped on the surface of the glass plate, pre-coated with bovine serum albumin (BSA). The ssDNA functionlized carbon nanotubes were allowed to dry overnight at room temperature (about 25 degrees centigrade). A fluorescein isothiocyanate (FITC) label was added to complimentary oligonucleotides, which were dropped on the ssDNA functionalized carbon nanotubes, which were heated to 60 degrees centigrade for 50 seconds. Afterwards the samples were washed and observed under a microscope, and hybridization is observed within 25 seconds. Atomic force microscopy was used to compare carbon nanotubes (A) before and (B) after ssDNA functionlization and after subsequent hybridization with (C) non-complementary DNA; and (D) hybridization with complementary DNA (cDNA), which shows that ssDNA functionalization and hybridization with cDNA takes place, when carbon nanotubes are used as a genetic probe (or marker). This provides a method for detection of DNA and RNA in a rapid test kit using bodily fluids or fluidized samples of DNA and RNA or fragments of DNA and RNA.

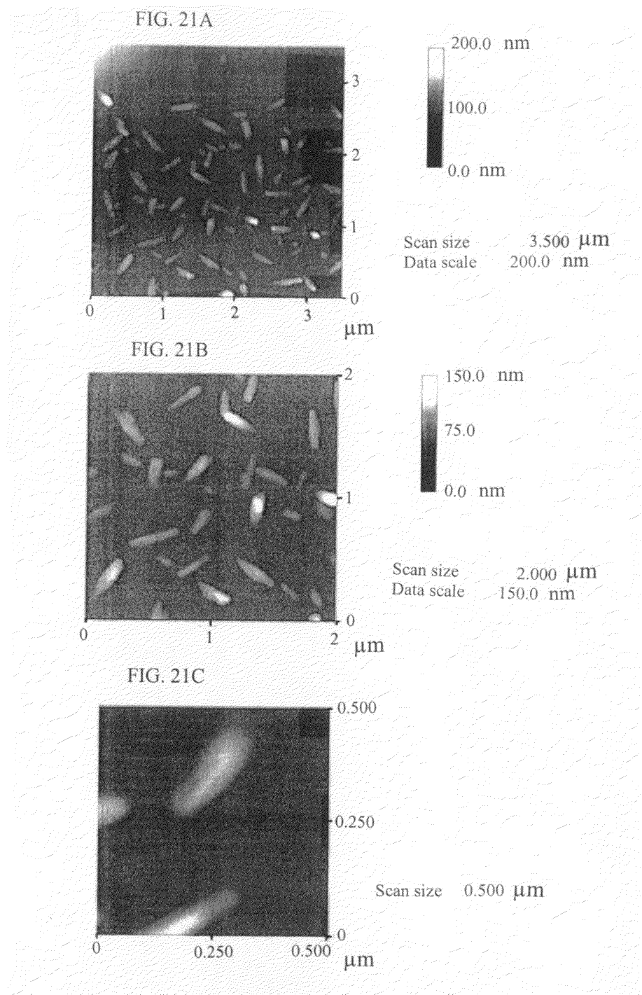

FIG. 21A-21C illustrate atomic force microscopy micrograph images of (A) carbon nanotubes (CNT), alone, without any ssDNA strand or fragment; (B) CNT functionalized by ssDNA; and (C) CNT functionalized by ssDNA and hybridization with a complementary DNA fragment, providing evidence of the hybridization process for preparing carbon nanotubes as a contrast marking agent for genetic probe test spots, for example. According to one example, any oligonucleotide having a complementary oligonucleotide may be hybridized with single wall carbon nanotubes according to the process disclosed.

In one example, a rapid test kit is capable of detecting samples of DNA, RNA or fragments thereof without the need of polymerase chain reactions to amplify the amount of DNA or RNA within a sample of bodily fluids. In another example, a small sample size may first be processed using a polymerase chain reaction technique to provide a sufficient concentration of DNA or RNA for detection by a rapid test kit. Preferably, no PCR is used, and the rapid test kit is capable of determining a qualitatitive comparison to a standard or quantitative viral load based on a measurable intensity, contrast or other physical property based on the concentration of particles, nanotubes or the like on a test region.

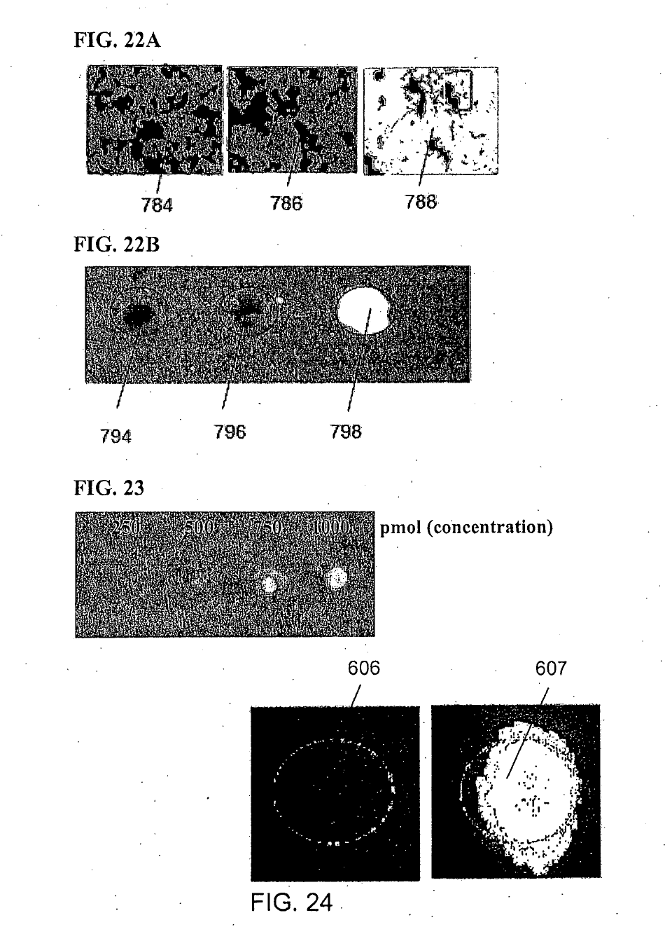

FIGS. 22A-B provide another example comparing non-complementary 786, 796 and complementary 788, 798 single strand oligonucleotides to a control 784, 794. The complementary oligonucleotide 788, 798 hybridizes the DNA, RNA or fragment of the DNA or RNA, resulting in fluorescence. Thus, the presence of complementary single strand oligonucleotides in a sample of blood is detectable by the hybridization. FIG. 23 illustrates a comparison of intensity with concentration of functionalized carbon nanotubes. A concentration of at least 750 pmol is preferred, but concentrations as low as 250 pmol are discernable.

In one example, a genetic probe is selected as a DNA primer for a viral RNA sequence, such as one of the viral RNA sequences for the viruses listed in the detailed disclosure. For example, one DNA primer may be coupled to a nanotube or nanoparticle, such as by thiolation and another DNA primer may be immobilized on a membrane, such as a cellulose filter membrane for one of the examples of rapid test kits using an antigen. In one example, both an antigen and a vrial RNA sequence may be detected to determine whether an individual has developed antibodies and a level or concentration of the viral load in the fluid sample volume tested. For example, a test kit may quickly determine if a regimen or treatment plan is not controlling an HIV infection, requiring immediate medical care or modification of a treatment plan.

Single strand oligonucleotides hybridization with gold nanoparticles was tested, also. Conjugates of gold nanoparticles and single strand oligonucleotides were prepared using citrate-stabilized gold nanoparticles and thiol-modified single strand oligonucleotides, as known in the art. For documenting surface coverage a 5′-fluorescein, 3′-thiol-labeled single strand oligonucleotide was used. Various single strand oligonucleotids may be selected having complementary oligonucleotides with oligonucleotide sequences to be detected, such as some of the following, with underling and double underline indicating oligonucleotide sequences for binding with complementary single strand oligonucleotides.

For example, a genetic probe is used in a point of care test. The point of care test used a rapid test kit that provides results of the test at room temperature. For example, an HIV test kit uses genetic probes for hybridizing one or more oligo nucleotides with HIV specific regions of the HIV RNA. For example, the LTR region of HIV has regions that are conserved among a wide variety of HIV strains, are unique to HIV (as compared to human DNA, for example), and are hybridizable at room temperature by an oligonucleotide probe. Some unique regions are identified in the following examples of HIV viral RNA sequences. Portions that are unique are identified below using a single underline.

HIV Unique Regions in Sequence Context of SEQ. ID. NO. 1:
GGTCTCTCTGGTTAGACCAGATCTGAGCCTGGGAGCTCTCTGGCTAACTAGGGAACCCACTGCTTAAGCC
TCAATAAAGCTTGCCTTGAGTGCTTCAAGTAGTGTGTGCCCGTCTGTTGTGTGACTCTGGTAACTAGAGA
TCCCTCAGACCCTTTTAGTCAGTGTGGAAAATCTCTAGCAGTGGCGCCCGAACAGGGACCTGAAAGCGAA
AGGGAAACCAGAGGAGCTCTCTCGACGCAGGACTCGGCTTGCTGAAGCGCGCACGGCAAGAGGCGAGGGG
CGGCGACTGGTGAGTACGCCAAAAATTTTGACTAGCGGAGGCTAGAAGGAGAGAGATGGGTGCGAGAGCG
TCAGTATTAAGCGGGGGAGAATTAGATCGATGGGAAAAAATTCGGTTAAGGCCAGGGGGAAAGAAAAAAT
ATAAATTAAAACATATAGTATGGGCAAGCAGGGAGCTAGAACGATTCGCAGTTAATCCTGGCCTGTTAGA
AACATCAGAAGGCTGTAGACAAATACTGGGACAGCTACAACCATCCCTTCAGACAGGATCAGAAGAACTT
AGATCATTATATAATACAGTAGCAACCCTCTATTGTGTGCATCAAAGGATAGAGATAAAAGACACCAAGG
AAGCTTTAGACAAGATAGAGGAAGAGCAAAACAAAAGTAAGAAAAAAGCACAGCAAGCAGCAGCTGACAC
AGGACACAGCAATCAGGTCAGCCAAAATTACCCTATAGTGCAGAACATCCAGGGGCAAATGGTACATCAG
GCCATATCACCTAGAACTTTAAATGCATGGGTAAAAGTAGTAGAAGAGAAGGCTTTCAGCCCAGAAGTGA
TACCCATGTTTTCAGCATTATCAGAAGGAGCCACCCCACAAGATTTAAACACCATGCTAAACACAGTGGG
GGGACATCAAGCAGCCATGCAAATGTTAAAAGAGACCATCAATGAGGAAGCTGCAGAATGGGATAGAGTG
CATCCAGTGCATGCAGGGCCTATTGCACCAGGCCAGATGAGAGAACCAAGGGGAAGTGACATAGCAGGAA
CTACTAGTACCCTTCAGGAACAAATAGGATGGATGACAAATAATCCACCTATCCCAGTAGGAGAAATTTA
TAAAAGATGGATAATCCTGGGATTAAATAAAATAGTAAGAATGTATAGCCCTACCAGCATTCTGGACATA
AGACAAGGACCAAAGGAACCCTTTAGAGACTATGTAGACCGGTTCTATAAAACTCTAAGAGCCGAGCAAG
CTTCACAGGAGGTAAAAAATTGGATGACAGAAACCTTGTTGGTCCAAAATGCGAACCCAGATTGTAAGAC
TATTTTAAAAGCATTGGGACCAGCGGCTACACTAGAAGAAATGATGACAGCATGTCAGGGAGTAGGAGGA
CCCGGCCATAAGGCAAGAGTTTTGGCTGAAGCAATGAGCCAAGTAACAAATTCAGCTACCATAATGATGC
AGAGAGGCAATTTTAGGAACCAAAGAAAGATTGTTAAGTGTTTCAATTGTGGCAAAGAAGGGCACACAGC
CAGAAATTGCAGGGCCCCTAGGAAAAAGGGCTGTTGGAAATGTGGAAAGGAAGGACACCAAATGAAAGAT
TGTACTGAGAGACAGGCTAATTTTTTAGGGAAGATCTGGCCTTCCTACAAGGGAAGGCCAGGGAATTTTC
TTCAGAGCAGACCAGAGCCAACAGCCCCACCAGAAGAGAGCTTCAGGTCTGGGGTAGAGACAACAACTCC
CCCTCAGAAGCAGGAGCCGATAGACAAGGAACTGTATCCTTTAACTTCCCTCAGGTCACTCTTTGGCAAC
GACCCCTCGTCACAATAAAGATAGGGGGGCAACTAAAGGAAGCTCTATTAGATACAGGAGCAGATGATAC
AGTATTAGAAGAAATGAGTTTGCCAGGAAGATGGAAACCAAAAATGATAGGGGGAATTGGAGGTTTTATC
AAAGTAAGACAGTATGATCAGATACTCATAGAAATCTGTGGACATAAAGCTATAGGTACAGTATTAGTAG
GACCTACACCTGTCAACATAATTGGAAGAAATCTGTTGACTCAGATTGGTTGCACTTTAAATTTTCCCAT
TAGCCCTATTGAGACTGTACCAGTAAAATTAAAGCCAGGAATGGATGGCCCAAAAGTTAAACAATGGCCA
TTGACAGAAGAAAAAATAAAAGCATTAGTAGAAATTTGTACAGAGATGGAAAAGGAAGGGAAAATTTCAA
AAATTGGGCCTGAAAATCCATACAATACTCCAGTATTTGCCATAAAGAAAAAAGACAGTACTAAATGGAG
AAAATTAGTAGATTTCAGAGAACTTAATAAGAGAACTCAAGACTTCTGGGAAGTTCAATTAGGAATACCA
CATCCCGCAGGGTTAAAAAAGAAAAAATCAGTAACAGTACTGGATGTGGGTGATGCATATTTTTCAGTTC
CCTTAGATGAAGACTTCAGGAAGTATACTGCATTTACCATACCTAGTATAAACAATGAGACACCAGGGAT
TAGATATCAGTACAATGTGCTTCCACAGGGATGGAAAGGATCACCAGCAATATTCCAAAGTAGCATGACA
AAAATCTTAGAGCCTTTTAGAAAACAAAATCCAGACATAGTTATCTATCAATACATGGATGATTTGTATG
TAGGATCTGACTTAGAAATAGGGCAGCATAGAACAAAAATAGAGGAGCTGAGACAACATCTGTTGAGGTG
GGGACTTACCACACCAGACAAAAAACATCAGAAAGAACCTCCATTCCTTTGGATGGGTTATGAACTCCAT
CCTGATAAATGGACAGTACAGCCTATAGTGCTGCCAGAAAAAGACAGCTGGACTGTCAATGACATACAGA
AGTTAGTGGGGAAATTGAATTGGGCAAGTCAGATTTACCCAGGGATTAAAGTAAGGCAATTATGTAAACT
CCTTAGAGGAACCAAAGCACTAACAGAAGTAATACCACTAACAGAAGAAGCAGAGCTAGAACTGGCAGAA
AACAGAGAGATTCTAAAAGAACCAGTACATGGAGTGTATTATGACCCATCAAAAGACTTAATAGCAGAAA
TACAGAAGCAGGGGCAAGGCCAATGGACATATCAAATTTATCAAGAGCCATTTAAAAATCTGAAAACAGG
AAAATATGCAAGAATGAGGGGTGCCCACACTAATGATGTAAAACAATTAACAGAGGCAGTGCAAAAAATA
ACCACAGAAAGCATAGTAATATGGGGAAAGACTCCTAAATTTAAACTGCCCATACAAAAGGAAACATGGG
AAACATGGTGGACAGAGTATTGGCAAGCCACCTGGATTCCTGAGTGGGAGTTTGTTAATACCCCTCCCTT
AGTGAAATTATGGTACCAGTTAGAGAAAGAACCCATAGTAGGAGCAGAAACCTTCTATGTAGATGGGGCA
GCTAACAGGGAGACTAAATTAGGAAAAGCAGGATATGTTACTAATAGAGGAAGACAAAAAGTTGTCACCC
TAACTGACACAACAAATCAGAAGACTGAGTTACAAGCAATTTATCTAGCTTTGCAGGATTCGGGATTAGA
AGTAAACATAGTAACAGACTCACAATATGCATTAGGAATCATTCAAGCACAACCAGATCAAAGTGAATCA
GAGTTAGTCAATCAAATAATAGAGCAGTTAATAAAAAAGGAAAAGGTCTATCTGGCATGGGTACCAGCAC
ACAAAGGAATTGGAGGAAATGAACAAGTAGATAAATTAGTCAGTGCTGGAATCAGGAAAGTACTATTTTT
AGATGGAATAGATAAGGCCCAAGATGAACATGAGAAATATCACAGTAATTGGAGAGCAATGGCTAGTGAT
TTTAACCTGCCACCTGTAGTAGCAAAAGAAATAGTAGCCAGCTGTGATAAATGTCAGCTAAAAGGAGAAG
CCATGCATGGACAAGTAGACTGTAGTCCAGGAATATGGCAACTAGATTGTACACATTTAGAAGGAAAAGT
TATCCTGGTAGCAGTTCATGTAGCCAGTGGATATATAGAAGCAGAAGTTATTCCAGCAGAAACAGGGCAG
GAAACAGCATATTTTCTTTTAAAATTAGCAGGAAGATGGCCAGTAAAAACAATACATACTGACAATGGCA
GCAATTTCACCGGTGCTACGGTTAGGGCCGCCTGTTGGTGGGCGGGAATCAAGCAGGAATTTGGAATTCC
CTACAATCCCCAAAGTCAAGGAGTAGTAGAATCTATGAATAAAGAATTAAAGAAAATTATAGGACAGGTA
AGAGATCAGGCTGAACATCTTAAGACAGCAGTACAAATGGCAGTATTCATCCACAATTTTAAAAGAAAAG
GGGGGATTGGGGGGTACAGTGCAGGGGAAAGAATAGTAGACATAATAGCAACAGACATACAAACTAAAGA
ATTACAAAAACAAATTACAAAAATTCAAAATTTTCGGGTTTATTACAGGGACAGCAGAAATCCACTTTGG
AAAGGACCAGCAAAGCTCCTCTGGAAAGGTGAAGGGGCAGTAGTAATACAAGATAATAGTGACATAAAAG
TAGTGCCAAGAAGAAAAGCAAAGATCATTAGGGATTATGGAAAACAGATGGCAGGTGATGATTGTGTGGC
AAGTAGACAGGATGAGGATTAGAACATGGAAAAGTTTAGTAAAACACCATATGTATGTTTCAGGGAAAGC
TAGGGGATGGTTTTATAGACATCACTATGAAAGCCCTCATCCAAGAATAAGTTCAGAAGTACACATCCCA
CTAGGGGATGCTAGATTGGTAATAACAACATATTGGGGTCTGCATACAGGAGAAAGAGACTGGCATTTGG
GTCAGGGAGTCTCCATAGAATGGAGGAAAAAGAGATATAGCACACAAGTAGACCCTGAACTAGCAGACCA
ACTAATTCATCTGTATTACTTTGACTGTTTTTCAGACTCTGCTATAAGAAAGGCCTTATTAGGACACATA
GTTAGCCCTAGGTGTGAATATCAAGCAGGACATAACAAGGTAGGATCTCTACAATACTTGGCACTAGCAG
CATTAATAACACCAAAAAAGATAAAGCCACCTTTGCCTAGTGTTACGAAACTGACAGAGGATAGATGGAA
CAAGCCCCAGAAGACCAAGGGCCACAGAGGGAGCCACACAATGAATGGACACTAGAGCTTTTAGAGGAGC
TTAAGAATGAAGCTGTTAGACATTTTCCTAGGATTTGGCTCCATGGCTTAGGGCAACATATCTATGAAAC
TTATGGGGATACTTGGGCAGGAGTGGAAGCCATAATAAGAATTCTGCAACAACTGCTGTTTATCCATTTT
CAGAATTGGGTGTCGACATAGCAGAATAGGCGTTACTCGACAGAGGAGAGCAAGAAATGGAGCCAGTAGA
TCCTAGACTAGAGCCCTGGAAGCATCCAGGAAGTCAGCCTAAAACTGCTTGTACCAATTGCTATTGTAAA
AAGTGTTGCTTTCATTGCCAAGTTTGTTTCATAACAAAAGCCTTAGGCATCTCCTATGGCAGGAAGAAGC
GGAGACAGCGACGAAGAGCTCATCAGAACAGTCAGACTCATCAAGCTTCTCTATCAAAGCAGTAAGTAGT
ACATGTAATGCAACCTATACCAATAGTAGCAATAGTAGCATTAGTAGTAGCAATAATAATAGCAATAGTT
GTGTGGTCCATAGTAATCATAGAATATAGGAAAATATTAAGACAAAGAAAAATAGACAGGTTAATTGATA
GACTAATAGAAAGAGCAGAAGACAGTGGCAATGAGAGTGAAGGAGAAATATCAGCACTTGTGGAGATGGG
GGTGGAGATGGGGCACCATGCTCCTTGGGATGTTGATGATCTGTAGTGCTACAGAAAAATTGTGGGTCAC
AGTCTATTATGGGGTACCTGTGTGGAAGGAAGCAACCACCACTCTATTTTGTGCATCAGATGCTAAAGCA
TATGATACAGAGGTACATAATGTTTGGGCCACACATGCCTGTGTACCCACAGACCCCAACCCACAAGAAG
TAGTATTGGTAAATGTGACAGAAAATTTTAACATGTGGAAAAATGACATGGTAGAACAGATGCATGAGGA
TATAATCAGTTTATGGGATCAAAGCCTAAAGCCATGTGTAAAATTAACCCCACTCTGTGTTAGTTTAAAG
TGCACTGATTTGAAGAATGATACTAATACCAATAGTAGTAGCGGGAGAATGATAATGGAGAAAGGAGAGA
TAAAAAACTGCTCTTTCAATATCAGCACAAGCATAAGAGGTAAGGTGCAGAAAGAATATGCATTTTTTTA
TAAACTTGATATAATACCAATAGATAATGATACTACCAGCTATAAGTTGACAAGTTGTAACACCTCAGTC
ATTACACAGGCCTGTCCAAAGGTATCCTTTGAGCCAATTCCCATACATTATTGTGCCCCGGCTGGTTTTG
CGATTCTAAAATGTAATAATAAGACGTTCAATGGAACAGGACCATGTACAAATGTCAGCACAGTACAATG
TACACATGGAATTAGGCCAGTAGTATCAACTCAACTGCTGTTAAATGGCAGTCTAGCAGAAGAAGAGGTA
GTAATTAGATCTGTCAATTTCACGGACAATGCTAAAACCATAATAGTACAGCTGAACACATCTGTAGAAA
TTAATTGTACAAGACCCAACAACAATACAAGAAAAAGAATCCGTATCCAGAGAGGACCAGGGAGAGCATT
TGTTACAATAGGAAAAATAGGAAATATGAGACAAGCACATTGTAACATTAGTAGAGCAAAATGGAATAAC
ACTTTAAAACAGATAGCTAGCAAATTAAGAGAACAATTTGGAAATAATAAAACAATAATCTTTAAGCAAT
CCTCAGGAGGGGACCCAGAAATTGTAACGCACAGTTTTAATTGTGGAGGGGAATTTTTCTACTGTAATTC
AACACAACTGTTTAATAGTACTTGGTTTAATAGTACTTGGAGTACTGAAGGGTCAAATAACACTGAAGGA
AGTGACACAATCACCCTCCCATGCAGAATAAAACAAATTATAAACATGTGGCAGAAAGTAGGAAAAGCAA
TGTATGCCCCTCCCATCAGTGGACAAATTAGATGTTCATCAAATATTACAGGGCTGCTATTAACAAGAGA
TGGTGGTAATAGCAACAATGAGTCCGAGATCTTCAGACCTGGAGGAGGAGATATGAGGGACAATTGGAGA
AGTGAATTATATAAATATAAAGTAGTAAAAATTGAACCATTAGGAGTAGCACCCACCAAGGCAAAGAGAA
GAGTGGTGCAGAGAGAAAAAAGAGCAGTGGGAATAGGAGCTTTGTTCCTTGGGTTCTTGGGAGCAGCAGG
AAGCACTATGGGCGGAGCCTCAATGACGCTGACGGTACAGGCCAGACAATTATTGTCTGGTATAGTGCAG
CAGCAGAACAATTTGCTGAGGGCTATTGAGGCGCAACAGCATCTGTTGCAACTCACAGTCTGGGGCATCA
AGCAGCTCCAGGCAAGAATCCTGGCTGTGGAAAGATACCTAAAGGATCAACAGCTCCTGGGGATTTGGGG
TTGCTCTGGAAAACTCATTTGCACCACTGCTGTGCCTTGGAATGCTAGTTGGAGTAATAAATCTCTGGAA
CAGATTTGGAATCACACGACCTGGATGGAGTGGGACAGAGAAATTAACAATTACACAAGCTTAATACACT
CCTTAATTGAAGAATCGCAAAACCAGCAAGAAAAGAATGAACAAGAATTATTGGAATTAGATAAATGGGC
AAGTTTGTGGAATTGGTTTAACATAACAAATTGGCTGTGGTATATAAAATTATTCATAATGATAGTAGGA
GGCTTGGTAGGTTTAAGAATAGTTTTTGCTGTACTTTCTATAGTGAATAGAGTTAGGCAGGGATATTCAC
CATTATCGTTTCAGACCCACCTCCCAACCCCGAGGGGACCCGACAGGCCCGAAGGAATAGAAGAAGAAGG
TGGAGAGAGAGACAGAGACAGATCCATTCGATTAGTGAACGGATCCTTGGCACTTATCTGGGACGATCTG
CGGAGCCTGTGCCTCTTCAGCTACCACCGCTTGAGAGACTTACTCTTGATTGTAACGAGGATTGTGGAAC
TTCTGGGACGCAGGGGGTGGGAAGCCCTCAAATATTGGTGGAATCTCCTACAGTATTGGAGTCAGGAACT
AAAGAATAGTGCTGTTAGCTTGCTCAATGCCACAGCCATAGCAGTAGCTGAGGGGACAGATAGGGTTATA
GAAGTAGTACAAGGAGCTTGTAGAGCTATTCGCCACATACCTAGAAGAATAAGACAGGGCTTGGAAAGGA
TTTTGCTATAAGATGGGTGGCAAGTGGTCAAAAAGTAGTGTGATTGGATGGCCTACTGTAAGGGAAAGAA
TGAGACGAGCTGAGCCAGCAGCAGATAGGGTGGGAGCAGCATCTCGAGACCTGGAAAAACATGGAGCAAT
CACAAGTAGCAATACAGCAGCTACCAATGCTGCTTGTGCCTGGCTAGAAGCACAAGAGGAGGAGGAGGTG
GGTTTTCCAGTCACACCTCAGGTACCTTTAAGACCAATGACTTACAAGGCAGCTGTAGATCTTAGCCACT
TTTTAAAAGAAAAGGGGGGACTGGAAGGGCTAATTCACTCCCAAAGAAGACAAGATATCCTTGATCTGTG
GATCTACCACACACAAGGCTACTTCCCTGATTAGCAGAACTACACACCAGGGCCAGGGGTCAGATATCCA
CTGACCTTTGGATGGTGCTACAAGCTAGTACCAGTTGAGCCAGATAAGATAGAAGAGGCCAATAAAGGAG
AGAACACCAGCTTGTTACACCCTGTGAGCCTGCATGGGATGGATGACCCGGAGAGAGAAGTGTTAGAGTG
GAGGTTTGACAGCCGCCTAGCATTTCATCACGTGGCCCGAGAGCTGCATCCGGAGTACTTCAAGAACTGC
TGACATCGAGCTTGCTACAAGGGACTTTCCGCTGGGGACTTTCCAGGGAGGCGTGGCCTGGGCGGGACTG
GGGAGTGGCGAGCCCTCAGATCCTGCATATAAGCAGCTGCTTTTTGCCTGTACTGGGTCTCTCTGGTTAG
ACCAGATCTGAGCCTGGGAGCTCTCTGGCTAACTAGGGAACCCACTGCTTAAGCCTCAATAAAGCTTGCC
TTGAGTGCTTC

A test kit usable at room temperature is most suitable for field and point of care testing. Ordinarily, hybridization of a genetic probe and viral RNA is not conducted at room temperature. The following portions of HIV RNA indicate in double underline certain portions identified as hybridizable at room temperature, for example.

HIV Unique Regions Identified for Point of Care Test for SEQ. ID. NO. 1:
GGTCTCTCTGGTTAGACCAGATCTGAGCCTGGGAGCTCTCTGGCTAACTAGGGAACCCACTGCTTAAGCC
TCAATAAAGCTTGCCTTGAGTGCTTCAAGTAGTGTGTGCCCGTCTGTTGTGTGACTCTGGTAACTAGAGA
TCCCTCAGACCCTTTTAGTCAGTGTGGAAAATCTCTAGCAGTGGCGCCCGAACAGGGACCTGAAAGCGAA
AGGGAAACCAGAGGAGCTCTCTCGACGCAGGACTCGGCTTGCTGAAGCGCGCACGGCAAGAGGCGAGGGG
CGGCGACTGGTGAGTACGCCAAAAATTTTGACTAGCGGAGGCTAGAAGGAGAGAGATGGGTGCGAGAGCG
TCAGTATTAAGCGGGGGAGAATTAGATCGATGGGAAAAAATTCGGTTAAGGCCAGGGGGAAAGAAAAAAT
ATAAATTAAAACATATAGTATGGGCAAGCAGGGAGCTAGAACGATTCGCAGTTAATCCTGGCCTGTTAGA
AACATCAGAAGGCTGTAGACAAATACTGGGACAGCTACAACCATCCCTTCAGACAGGATCAGAAGAACTT
AGATCATTATATAATACAGTAGCAACCCTCTATTGTGTGCATCAAAGGATAGAGATAAAAGACACCAAGG
AAGCTTTAGACAAGATAGAGGAAGAGCAAAACAAAAGTAAGAAAAAAGCACAGCAAGCAGCAGCTGACAC
AGGACACAGCAATCAGGTCAGCCAAAATTACCCTATAGTGCAGAACATCCAGGGGCAAATGGTACATCAG
GCCATATCACCTAGAACTTTAAATGCATGGGTAAAAGTAGTAGAAGAGAAGGCTTTCAGCCCAGAAGTGA
TACCCATGTTTTCAGCATTATCAGAAGGAGCCACCCCACAAGATTTAAACACCATGCTAAACACAGTGGG
GGGACATCAAGCAGCCATGCAAATGTTAAAAGAGACCATCAATGAGGAAGCTGCAGAATGGGATAGAGTG
CATCCAGTGCATGCAGGGCCTATTGCACCAGGCCAGATGAGAGAACCAAGGGGAAGTGACATAGCAGGAA
CTACTAGTACCCTTCAGGAACAAATAGGATGGATGACAAATAATCCACCTATCCCAGTAGGAGAAATTTA
TAAAAGATGGATAATCCTGGGATTAAATAAAATAGTAAGAATGTATAGCCCTACCAGCATTCTGGACATA
AGACAAGGACCAAAGGAACCCTTTAGAGACTATGTAGACCGGTTCTATAAAACTCTAAGAGCCGAGCAAG
CTTCACAGGAGGTAAAAAATTGGATGACAGAAACCTTGTTGGTCCAAAATGCGAACCCAGATTGTAAGAC
TATTTTAAAAGCATTGGGACCAGCGGCTACACTAGAAGAAATGATGACAGCATGTCAGGGAGTAGGAGGA
CCCGGCCATAAGGCAAGAGTTTTGGCTGAAGCAATGAGCCAAGTAACAAATTCAGCTACCATAATGATGC
AGAGAGGCAATTTTAGGAACCAAAGAAAGATTGTTAAGTGTTTCAATTGTGGCAAAGAAGGGCACACAGC
CAGAAATTGCAGGGCCCCTAGGAAAAAGGGCTGTTGGAAATGTGGAAAGGAAGGACACCAAATGAAAGAT
TGTACTGAGAGACAGGCTAATTTTTTAGGGAAGATCTGGCCTTCCTACAAGGGAAGGCCAGGGAATTTTC
TTCAGAGCAGACCAGAGCCAACAGCCCCACCAGAAGAGAGCTTCAGGTCTGGGGTAGAGACAACAACTCC
CCCTCAGAAGCAGGAGCCGATAGACAAGGAACTGTATCCTTTAACTTCCCTCAGGTCACTCTTTGGCAAC
GACCCCTCGTCACAATAAAGATAGGGGGGCAACTAAAGGAAGCTCTATTAGATACAGGAGCAGATGATAC
AGTATTAGAAGAAATGAGTTTGCCAGGAAGATGGAAACCAAAAATGATAGGGGGAATTGGAGGTTTTATC
AAAGTAAGACAGTATGATCAGATACTCATAGAAATCTGTGGACATAAAGCTATAGGTACAGTATTAGTAG
GACCTACACCTGTCAACATAATTGGAAGAAATCTGTTGACTCAGATTGGTTGCACTTTAAATTTTCCCAT
TAGCCCTATTGAGACTGTACCAGTAAAATTAAAGCCAGGAATGGATGGCCCAAAAGTTAAACAATGGCCA
TTGACAGAAGAAAAAATAAAAGCATTAGTAGAAATTTGTACAGAGATGGAAAAGGAAGGGAAAATTTCAA
AAATTGGGCCTGAAAATCCATACAATACTCCAGTATTTGCCATAAAGAAAAAAGACAGTACTAAATGGAG
AAAATTAGTAGATTTCAGAGAACTTAATAAGAGAACTCAAGACTTCTGGGAAGTTCAATTAGGAATACCA
CATCCCGCAGGGTTAAAAAAGAAAAAATCAGTAACAGTACTGGATGTGGGTGATGCATATTTTTCAGTTC
CCTTAGATGAAGACTTCAGGAAGTATACTGCATTTACCATACCTAGTATAAACAATGAGACACCAGGGAT
TAGATATCAGTACAATGTGCTTCCACAGGGATGGAAAGGATCACCAGCAATATTCCAAAGTAGCATGACA
AAAATCTTAGAGCCTTTTAGAAAACAAAATCCAGACATAGTTATCTATCAATACATGGATGATTTGTATG
TAGGATCTGACTTAGAAATAGGGCAGCATAGAACAAAAATAGAGGAGCTGAGACAACATCTGTTGAGGTG
GGGACTTACCACACCAGACAAAAAACATCAGAAAGAACCTCCATTCCTTTGGATGGGTTATGAACTCCAT
CCTGATAAATGGACAGTACAGCCTATAGTGCTGCCAGAAAAAGACAGCTGGACTGTCAATGACATACAGA
AGTTAGTGGGGAAATTGAATTGGGCAAGTCAGATTTACCCAGGGATTAAAGTAAGGCAATTATGTAAACT
CCTTAGAGGAACCAAAGCACTAACAGAAGTAATACCACTAACAGAAGAAGCAGAGCTAGAACTGGCAGAA
AACAGAGAGATTCTAAAAGAACCAGTACATGGAGTGTATTATGACCCATCAAAAGACTTAATAGCAGAAA
TACAGAAGCAGGGGCAAGGCCAATGGACATATCAAATTTATCAAGAGCCATTTAAAAATCTGAAAACAGG
AAAATATGCAAGAATGAGGGGTGCCCACACTAATGATGTAAAACAATTAACAGAGGCAGTGCAAAAAATA
ACCACAGAAAGCATAGTAATATGGGGAAAGACTCCTAAATTTAAACTGCCCATACAAAAGGAAACATGGG
AAACATGGTGGACAGAGTATTGGCAAGCCACCTGGATTCCTGAGTGGGAGTTTGTTAATACCCCTCCCTT
AGTGAAATTATGGTACCAGTTAGAGAAAGAACCCATAGTAGGAGCAGAAACCTTCTATGTAGATGGGGCA
GCTAACAGGGAGACTAAATTAGGAAAAGCAGGATATGTTACTAATAGAGGAAGACAAAAAGTTGTCACCC
TAACTGACACAACAAATCAGAAGACTGAGTTACAAGCAATTTATCTAGCTTTGCAGGATTCGGGATTAGA
AGTAAACATAGTAACAGACTCACAATATGCATTAGGAATCATTCAAGCACAACCAGATCAAAGTGAATCA
GAGTTAGTCAATCAAATAATAGAGCAGTTAATAAAAAAGGAAAAGGTCTATCTGGCATGGGTACCAGCAC
ACAAAGGAATTGGAGGAAATGAACAAGTAGATAAATTAGTCAGTGCTGGAATCAGGAAAGTACTATTTTT
AGATGGAATAGATAAGGCCCAAGATGAACATGAGAAATATCACAGTAATTGGAGAGCAATGGCTAGTGAT
TTTAACCTGCCACCTGTAGTAGCAAAAGAAATAGTAGCCAGCTGTGATAAATGTCAGCTAAAAGGAGAAG
CCATGCATGGACAAGTAGACTGTAGTCCAGGAATATGGCAACTAGATTGTACACATTTAGAAGGAAAAGT
TATCCTGGTAGCAGTTCATGTAGCCAGTGGATATATAGAAGCAGAAGTTATTCCAGCAGAAACAGGGCAG
GAAACAGCATATTTTCTTTTAAAATTAGCAGGAAGATGGCCAGTAAAAACAATACATACTGACAATGGCA
GCAATTTCACCGGTGCTACGGTTAGGGCCGCCTGTTGGTGGGCGGGAATCAAGCAGGAATTTGGAATTCC
CTACAATCCCCAAAGTCAAGGAGTAGTAGAATCTATGAATAAAGAATTAAAGAAAATTATAGGACAGGTA
AGAGATCAGGCTGAACATCTTAAGACAGCAGTACAAATGGCAGTATTCATCCACAATTTTAAAAGAAAAG
GGGGGATTGGGGGGTACAGTGCAGGGGAAAGAATAGTAGACATAATAGCAACAGACATACAAACTAAAGA
ATTACAAAAACAAATTACAAAAATTCAAAATTTTCGGGTTTATTACAGGGACAGCAGAAATCCACTTTGG
AAAGGACCAGCAAAGCTCCTCTGGAAAGGTGAAGGGGCAGTAGTAATACAAGATAATAGTGACATAAAAG
TAGTGCCAAGAAGAAAAGCAAAGATCATTAGGGATTATGGAAAACAGATGGCAGGTGATGATTGTGTGGC
AAGTAGACAGGATGAGGATTAGAACATGGAAAAGTTTAGTAAAACACCATATGTATGTTTCAGGGAAAGC
TAGGGGATGGTTTTATAGACATCACTATGAAAGCCCTCATCCAAGAATAAGTTCAGAAGTACACATCCCA
CTAGGGGATGCTAGATTGGTAATAACAACATATTGGGGTCTGCATACAGGAGAAAGAGACTGGCATTTGG
GTCAGGGAGTCTCCATAGAATGGAGGAAAAAGAGATATAGCACACAAGTAGACCCTGAACTAGCAGACCA
ACTAATTCATCTGTATTACTTTGACTGTTTTTCAGACTCTGCTATAAGAAAGGCCTTATTAGGACACATA
GTTAGCCCTAGGTGTGAATATCAAGCAGGACATAACAAGGTAGGATCTCTACAATACTTGGCACTAGCAG
CATTAATAACACCAAAAAAGATAAAGCCACCTTTGCCTAGTGTTACGAAACTGACAGAGGATAGATGGAA
CAAGCCCCAGAAGACCAAGGGCCACAGAGGGAGCCACACAATGAATGGACACTAGAGCTTTTAGAGGAGC
TTAAGAATGAAGCTGTTAGACATTTTCCTAGGATTTGGCTCCATGGCTTAGGGCAACATATCTATGAAAC
TTATGGGGATACTTGGGCAGGAGTGGAAGCCATAATAAGAATTCTGCAACAACTGCTGTTTATCCATTTT
CAGAATTGGGTGTCGACATAGCAGAATAGGCGTTACTCGACAGAGGAGAGCAAGAAATGGAGCCAGTAGA
TCCTAGACTAGAGCCCTGGAAGCATCCAGGAAGTCAGCCTAAAACTGCTTGTACCAATTGCTATTGTAAA
AAGTGTTGCTTTCATTGCCAAGTTTGTTTCATAACAAAAGCCTTAGGCATCTCCTATGGCAGGAAGAAGC
GGAGACAGCGACGAAGAGCTCATCAGAACAGTCAGACTCATCAAGCTTCTCTATCAAAGCAGTAAGTAGT
ACATGTAATGCAACCTATACCAATAGTAGCAATAGTAGCATTAGTAGTAGCAATAATAATAGCAATAGTT
GTGTGGTCCATAGTAATCATAGAATATAGGAAAATATTAAGACAAAGAAAAATAGACAGGTTAATTGATA
GACTAATAGAAAGAGCAGAAGACAGTGGCAATGAGAGTGAAGGAGAAATATCAGCACTTGTGGAGATGGG
GGTGGAGATGGGGCACCATGCTCCTTGGGATGTTGATGATCTGTAGTGCTACAGAAAAATTGTGGGTCAC
AGTCTATTATGGGGTACCTGTGTGGAAGGAAGCAACCACCACTCTATTTTGTGCATCAGATGCTAAAGCA
TATGATACAGAGGTACATAATGTTTGGGCCACACATGCCTGTGTACCCACAGACCCCAACCCACAAGAAG
TAGTATTGGTAAATGTGACAGAAAATTTTAACATGTGGAAAAATGACATGGTAGAACAGATGCATGAGGA
TATAATCAGTTTATGGGATCAAAGCCTAAAGCCATGTGTAAAATTAACCCCACTCTGTGTTAGTTTAAAG
TGCACTGATTTGAAGAATGATACTAATACCAATAGTAGTAGCGGGAGAATGATAATGGAGAAAGGAGAGA
TAAAAAACTGCTCTTTCAATATCAGCACAAGCATAAGAGGTAAGGTGCAGAAAGAATATGCATTTTTTTA
TAAACTTGATATAATACCAATAGATAATGATACTACCAGCTATAAGTTGACAAGTTGTAACACCTCAGTC
ATTACACAGGCCTGTCCAAAGGTATCCTTTGAGCCAATTCCCATACATTATTGTGCCCCGGCTGGTTTTG
CGATTCTAAAATGTAATAATAAGACGTTCAATGGAACAGGACCATGTACAAATGTCAGCACAGTACAATG
TACACATGGAATTAGGCCAGTAGTATCAACTCAACTGCTGTTAAATGGCAGTCTAGCAGAAGAAGAGGTA
GTAATTAGATCTGTCAATTTCACGGACAATGCTAAAACCATAATAGTACAGCTGAACACATCTGTAGAAA
TTAATTGTACAAGACCCAACAACAATACAAGAAAAAGAATCCGTATCCAGAGAGGACCAGGGAGAGCATT
TGTTACAATAGGAAAAATAGGAAATATGAGACAAGCACATTGTAACATTAGTAGAGCAAAATGGAATAAC
ACTTTAAAACAGATAGCTAGCAAATTAAGAGAACAATTTGGAAATAATAAAACAATAATCTTTAAGCAAT
CCTCAGGAGGGGACCCAGAAATTGTAACGCACAGTTTTAATTGTGGAGGGGAATTTTTCTACTGTAATTC
AACACAACTGTTTAATAGTACTTGGTTTAATAGTACTTGGAGTACTGAAGGGTCAAATAACACTGAAGGA
AGTGACACAATCACCCTCCCATGCAGAATAAAACAAATTATAAACATGTGGCAGAAAGTAGGAAAAGCAA
TGTATGCCCCTCCCATCAGTGGACAAATTAGATGTTCATCAAATATTACAGGGCTGCTATTAACAAGAGA
TGGTGGTAATAGCAACAATGAGTCCGAGATCTTCAGACCTGGAGGAGGAGATATGAGGGACAATTGGAGA
AGTGAATTATATAAATATAAAGTAGTAAAAATTGAACCATTAGGAGTAGCACCCACCAAGGCAAAGAGAA
GAGTGGTGCAGAGAGAAAAAAGAGCAGTGGGAATAGGAGCTTTGTTCCTTGGGTTCTTGGGAGCAGCAGG
AAGCACTATGGGCGCAGCCTCAATGACGCTGACGGTACAGGCCAGACAATTATTGTCTGGTATAGTGCAG
CAGCAGAACAATTTGCTGAGGGCTATTGAGGCGCAACAGCATCTGTTGCAACTCACAGTCTGGGGCATCA
AGCAGCTCCAGGCAAGAATCCTGGCTGTGGAAAGATACCTAAAGGATCAACAGCTCCTGGGGATTTGGGG
TTGCTCTGGAAAACTCATTTGCACCACTGCTGTGCCTTGGAATGCTAGTTGGAGTAATAAATCTCTGGAA
CAGATTTGGAATCACACGACCTGGATGGAGTGGGACAGAGAAATTAACAATTACACAAGCTTAATACACT
CCTTAATTGAAGAATCGCAAAACCAGCAAGAAAAGAATGAACAAGAATTATTGGAATTAGATAAATGGGC
AAGTTTGTGGAATTGGTTTAACATAACAAATTGGCTGTGGTATATAAAATTATTCATAATGATAGTAGGA
GGCTTGGTAGGTTTAAGAATAGTTTTTGCTGTACTTTCTATAGTGAATAGAGTTAGGCAGGGATATTCAC
CATTATCGTTTCAGACCCACCTCCCAACCCCGAGGGGACCCGACAGGCCCGAAGGAATAGAAGAAGAAGG
TGGAGAGAGAGACAGAGACAGATCCATTCGATTAGTGAACGGATCCTTGGCACTTATCTGGGACGATCTG
CGGAGCCTGTGCCTCTTCAGCTACCACCGCTTGAGAGACTTACTCTTGATTGTAACGAGGATTGTGGAAC
TTCTGGGACGCAGGGGGTGGGAAGCCCTCAAATATTGGTGGAATCTCCTACAGTATTGGAGTCAGGAACT
AAAGAATAGTGCTGTTAGCTTGCTCAATGCCACAGCCATAGCAGTAGCTGAGGGGACAGATAGGGTTATA
GAAGTAGTACAAGGAGCTTGTAGAGCTATTCGCCACATACCTAGAAGAATAAGACAGGGCTTGGAAAGGA
TTTTGCTATAAGATGGGTGGCAAGTGGTCAAAAAGTAGTGTGATTGGATGGCCTACTGTAAGGGAAAGAA
TGAGACGAGCTGAGCCAGCAGCAGATAGGGTGGGAGCAGCATCTCGAGACCTGGAAAAACATGGAGCAAT
CACAAGTAGCAATACAGCAGCTACCAATGCTGCTTGTGCCTGGCTAGAAGCACAAGAGGAGGAGGAGGTG
GGTTTTCCAGTCACACCTCAGGTACCTTTAAGACCAATGACTTACAAGGCAGCTGTAGATCTTAGCCACT
TTTTAAAAGAAAAGGGGGGACTGGAAGGGCTAATTCACTCCCAAAGAAGACAAGATATCCTTGATCTGTG
GATCTACCACACACAAGGCTACTTCCCTGATTAGCAGAACTACACACCAGGGCCAGGGGTCAGATATCCA
CTGACCTTTGGATGGTGCTACAAGCTAGTACCAGTTGAGCCAGATAAGATAGAAGAGGCCAATAAAGGAG
AGAACACCAGCTTGTTACACCCTGTGAGCCTGCATGGGATGGATGACCCGGAGAGAGAAGTGTTAGAGTG
GAGGTTTGACAGCCGCCTAGCATTTCATCACGTGGCCCGAGAGCTGCATCCGGAGTACTTCAAGAACTGC
TGACATCGAGCTTGCTACAAGGGACTTTCCGCTGGGGACTTTCCAGGGAGGCGTGGCCTGGGCGGGACTG
GGGAGTGGCGAGCCCTCAGATCCTGCATATAAGCAGCTGCTTTTTGCCTGTACTGGGTCTCTCTGGTTAG
ACCAGATCTGAGCCTGGGAGCTCTCTGGCTAACTAGGGAACCCACTGCTTAAGCCTCAATAAAGCTTGCC
TTGAGTGCTTC

Specific oligonucleotide sequences suitable for point of care test kits are provided in the following examples:

SEQ. ID. NO. 2, Sequence 1-44 (Before GAG region)
GGTCTCTCTGGTTAGACCAGATCTGAGCCTGGGAGCTCTCTGGC
SEQ. ID. NO. 3, Sequence 63-179 (Before GAG region)
CTTAAGCCTCAATAAAGCTTGCCTTGAGTGCTTCAAGTAGTGTGTGCCCGTCTGTTGTGTGACTCTGGTAACTAGAGA
TCCCTCAGACCCTTTTAGTCAGTGTGGAAAATCTCTAGC
SEQ. ID. NO. 4, Sequence 252-364 (Sequence before and within GAG region)
CTGAAGCGCGCACGGCAAGAGGCGAGGGGCGGCGACTGGTGAGTACGCCAAAAATTTTGACTAGCGGAGGCTAG
AAGGAGAGAGATGGGTGCGAGAGCGTCAGTATTAAGCGG
SEQ. ID. NO. 5, Sequence 448-539 (Sequence in the GAG region)
GCAGGGAGCTAGAACGATTCGCAGTTAATCCTGGCCTGTTAGAAACATCAGAAGGCTGTAGACAAATACTGGGACA
GCTACAACCATCCCTT
SEQ. ID. NO. 6, Sequence 762-929 (Sequence in the GAG region)
GTACATCAGGCCATATCACCTAGAACTTTAAATGCATGGGTAAAAGTAGTAGAAGAGAAGGCTTTCAGCCCAGAAG
TGATACCCATGTTTTCAGCATTATCAGAAGGAGCCACCCCACAAGATTTAAACACCATGCTAAACACAGTGGGGGGA
CATCAAGCAGCCATG
SEQ. ID. NO. 7, Sequence 951-1113 (Sequence in the GAG region)
AATGAGGAAGCTGCAGAATGGGATAGAGTGCATCCAGTGCATGCAGGGCCTATTGCACCAGGCCAGATGAGAGAA
CCAAGGGGAAGTGACATAGCAGGAACTACTAGTACCCTTCAGGAACAAATAGGATGGATGACAAATAATCCACCTA
TCCCAGTAGGAG
SEQ. ID. NO. 8, Sequence 1197-1255 (Sequence in the GAG-POL region)
GGACCAAAGGAACCCTTTAGAGACTATGTAGACCGGTTCTATAAAACTCTAAGAGCCGA
SEQ. ID. NO. 9, Sequence 1378-1422 (Sequence in the GAG-POL region)
CAGCATGTCAGGGAGTAGGAGGACCCGGCCATAAGGCAAGAGTTT
SEQ. ID. NO. 10, Sequence 2873-2930 (Sequence in the GAG-POL region)
TTAGTGGGGAAATTGAATTGGGCAAGTCAGATTTACCCAGGGATTAAAGTAAGGCAAT
SEQ. ID. NO. 11, Sequence 8464-8515 (Sequence in the NEF region)
GAGCAATCACAAGTAGCAATACAGCAGCTACCAATGCTGCTTGTGCCTGGCT
SEQ. ID. NO. 12, Sequence 8880-9001 (Sequence within and after NEF region)
GTGTTAGAGTGGAGGTTTGACAGCCGCCTAGCATTTCATCACGTGGCCCGAGAGCTGCATCCGGAGTACTTCAAGA
ACTGCTGACATCGAGCTTGCTACAAGGGACTTTCCGCTGGGGACTT
SEQ. ID. NO. 13, Sequence 9077-9129 (Sequence after NEF region)
CCTGTACTGGGTCTCTCTGGTTAGACCAGATCTGAGCCTGGGAGCTCTCTGGC

Some additional examples of unique portions of HIV viral RNA are identified in the following sequence of SEQ. ID. NO. 1 by single and double underline for use in hybridization and detection of HIV viral RNA with a genetic probe:

GGTCTCTCTGGTTAGACCAGATCTGAGCCTGGGAGCTCTCTGGCTAACTAGGGAACCCACTGCTTAAGCC
TCAATAAAGCTTGCCTTGAGTGCTTCAAGTAGTGTGTGCCCGTCTGTTGTGTGACTCTGGTAACTAGAGA
TCCCTCAGACCCTTTTAGTCAGTGTGGAAAATCTCTAGCAGTGGCGCCCGAACAGGGACCTGAAAGCGAA
AGGGAAACCAGAGGAGCTCTCTCGACGCAGGACTCGGCTTGCTGAAGCGCGCACGGCAAGAGGCGAGGGG
CGGCGACTGGTGAGTACGCCAAAAATTTTGACTAGCGGAGGCTAGAAGGAGAGAGATGGGTGCGAGAGCG
TCAGTATTAAGCGGGGGAGAATTAGATCGATGGGAAAAAATTCGGTTAAGGCCAGGGGGAAAGAAAAAAT
ATAAATTAAAACATATAGTATGGGCAAGCAGGGAGCTAGAACGATTCGCAGTTAATCCTGGCCTGTTAGA
AACATCAGAAGGCTGTAGACAAATACTGGGACAGCTACAACCATCCCTTCAGACAGGATCAGAAGAACTT
AGATCATTATATAATACAGTAGCAACCCTCTATTGTGTGCATCAAAGGATAGAGATAAAAGACACCAAGG
AAGCTTTAGACAAGATAGAGGAAGAGCAAAACAAAAGTAAGAAAAAAGCACAGCAAGCAGCAGCTGACAC
AGGACACAGCAATCAGGTCAGCCAAAATTACCCTATAGTGCAGAACATCCAGGGGCAAATGGTACATCAG
GCCATATCACCTAGAACTTTAAATGCATGGGTAAAAGTAGTAGAAGAGAAGGCTTTCAGCCCAGAAGTGA
TACCCATGTTTTCAGCATTATCAGAAGGAGCCACCCCACAAGATTTAAACACCATGCTAAACACAGTGGG
GGGACATCAAGCAGCCATGCAAATGTTAAAAGAGACCATCAATGAGGAAGCTGCAGAATGGGATAGAGTG
CATCCAGTGCATGCAGGGCCTATTGCACCAGGCCAGATGAGAGAACCAAGGGGAAGTGACATAGCAGGAA
CTACTAGTACCCTTCAGGAACAAATAGGATGGATGACAAATAATCCACCTATCCCAGTAGGAGAAATTTA
TAAAAGATGGATAATCCTGGGATTAAATAAAATAGTAAGAATGTATAGCCCTACCAGCATTCTGGACATA
AGACAAGGACCAAAGGAACCCTTTAGAGACTATGTAGACCGGTTCTATAAAACTCTAAGAGCCGAGCAAG
CTTCACAGGAGGTAAAAAATTGGATGACAGAAACCTTGTTGGTCCAAAATGCGAACCCAGATTGTAAGAC
TATTTTAAAAGCATTGGGACCAGCGGCTACACTAGAAGAAATGATGACAGCATGTCAGGGAGTAGGAGGA
CCCGGCCATAAGGCAAGAGTTTTGGCTGAAGCAATGAGCCAAGTAACAAATTCAGCTACCATAATGATGC
AGAGAGGCAATTTTAGGAACCAAAGAAAGATTGTTAAGTGTTTCAATTGTGGCAAAGAAGGGCACACAGC
CAGAAATTGCAGGGCCCCTAGGAAAAAGGGCTGTTGGAAATGTGGAAAGGAAGGACACCAAATGAAAGAT
TGTACTGAGAGACAGGCTAATTTTTTAGGGAAGATCTGGCCTTCCTACAAGGGAAGGCCAGGGAATTTTC
TTCAGAGCAGACCAGAGCCAACAGCCCCACCAGAAGAGAGCTTCAGGTCTGGGGTAGAGACAACAACTCC
CCCTCAGAAGCAGGAGCCGATAGACAAGGAACTGTATCCTTTAACTTCCCTCAGGTCACTCTTTGGCAAC
GACCCCTCGTCACAATAAAGATAGGGGGGCAACTAAAGGAAGCTCTATTAGATACAGGAGCAGATGATAC
AGTATTAGAAGAAATGAGTTTGCCAGGAAGATGGAAACCAAAAATGATAGGGGGAATTGGAGGTTTTATC
AAAGTAAGACAGTATGATCAGATACTCATAGAAATCTGTGGACATAAAGCTATAGGTACAGTATTAGTAG
GACCTACACCTGTCAACATAATTGGAAGAAATCTGTTGACTCAGATTGGTTGCACTTTAAATTTTCCCAT
TAGCCCTATTGAGACTGTACCAGTAAAATTAAAGCCAGGAATGGATGGCCCAAAAGTTAAACAATGGCCA
TTGACAGAAGAAAAAATAAAAGCATTAGTAGAAATTTGTACAGAGATGGAAAAGGAAGGGAAAATTTCAA
AAATTGGGCCTGAAAATCCATACAATACTCCAGTATTTGCCATAAAGAAAAAAGACAGTACTAAATGGAG
AAAATTAGTAGATTTCAGAGAACTTAATAAGAGAACTCAAGACTTCTGGGAAGTTCAATTAGGAATACCA
CATCCCGCAGGGTTAAAAAAGAAAAAATCAGTAACAGTACTGGATGTGGGTGATGCATATTTTTCAGTTC
CCTTAGATGAAGACTTCAGGAAGTATACTGCATTTACCATACCTAGTATAAACAATGAGACACCAGGGAT
TAGATATCAGTACAATGTGCTTCCACAGGGATGGAAAGGATCACCAGCAATATTCCAAAGTAGCATGACA
AAAATCTTAGAGCCTTTTAGAAAACAAAATCCAGACATAGTTATCTATCAATACATGGATGATTTGTATG
TAGGATCTGACTTAGAAATAGGGCAGCATAGAACAAAAATAGAGGAGCTGAGACAACATCTGTTGAGGTG
GGGACTTACCACACCAGACAAAAAACATCAGAAAGAACCTCCATTCCTTTGGATGGGTTATGAACTCCAT
CCTGATAAATGGACAGTACAGCCTATAGTGCTGCCAGAAAAAGACAGCTGGACTGTCAATGACATACAGA
AGTTAGTGGGGAAATTGAATTGGGCAAGTCAGATTTACCCAGGGATTAAAGTAAGGCAATTATGTAAACT
CCTTAGAGGAACCAAAGCACTAACAGAAGTAATACCACTAACAGAAGAAGCAGAGCTAGAACTGGCAGAA
AACAGAGAGATTCTAAAAGAACCAGTACATGGAGTGTATTATGACCCATCAAAAGACTTAATAGCAGAAA
TACAGAAGCAGGGGCAAGGCCAATGGACATATCAAATTTATCAAGAGCCATTTAAAAATCTGAAAACAGG
AAAATATGCAAGAATGAGGGGTGCCCACACTAATGATGTAAAACAATTAACAGAGGCAGTGCAAAAAATA
ACCACAGAAAGCATAGTAATATGGGGAAAGACTCCTAAATTTAAACTGCCCATACAAAAGGAAACATGGG
AAACATGGTGGACAGAGTATTGGCAAGCCACCTGGATTCCTGAGTGGGAGTTTGTTAATACCCCTCCCTT
AGTGAAATTATGGTACCAGTTAGAGAAAGAACCCATAGTAGGAGCAGAAACCTTCTATGTAGATGGGGCA
GCTAACAGGGAGACTAAATTAGGAAAAGCAGGATATGTTACTAATAGAGGAAGACAAAAAGTTGTCACCC
TAACTGACACAACAAATCAGAAGACTGAGTTACAAGCAATTTATCTAGCTTTGCAGGATTCGGGATTAGA
AGTAAACATAGTAACAGACTCACAATATGCATTAGGAATCATTCAAGCACAACCAGATCAAAGTGAATCA
GAGTTAGTCAATCAAATAATAGAGCAGTTAATAAAAAAGGAAAAGGTCTATCTGGCATGGGTACCAGCAC
ACAAAGGAATTGGAGGAAATGAACAAGTAGATAAATTAGTCAGTGCTGGAATCAGGAAAGTACTATTTTT
AGATGGAATAGATAAGGCCCAAGATGAACATGAGAAATATCACAGTAATTGGAGAGCAATGGCTAGTGAT
TTTAACCTGCCACCTGTAGTAGCAAAAGAAATAGTAGCCAGCTGTGATAAATGTCAGCTAAAAGGAGAAG
CCATGCATGGACAAGTAGACTGTAGTCCAGGAATATGGCAACTAGATTGTACACATTTAGAAGGAAAAGT
TATCCTGGTAGCAGTTCATGTAGCCAGTGGATATATAGAAGCAGAAGTTATTCCAGCAGAAACAGGGCAG
GAAACAGCATATTTTCTTTTAAAATTAGCAGGAAGATGGCCAGTAAAAACAATACATACTGACAATGGCA
GCAATTTCACCGGTGCTACGGTTAGGGCCGCCTGTTGGTGGGCGGGAATCAAGCAGGAATTTGGAATTCC
CTACAATCCCCAAAGTCAAGGAGTAGTAGAATCTATGAATAAAGAATTAAAGAAAATTATAGGACAGGTA
AGAGATCAGGCTGAACATCTTAAGACAGCAGTACAAATGGCAGTATTCATCCACAATTTTAAAAGAAAAG
GGGGGATTGGGGGGTACAGTGCAGGGGAAAGAATAGTAGACATAATAGCAACAGACATACAAACTAAAGA
ATTACAAAAACAAATTACAAAAATTCAAAATTTTCGGGTTTATTACAGGGACAGCAGAAATCCACTTTGG
AAAGGACCAGCAAAGCTCCTCTGGAAAGGTGAAGGGGCAGTAGTAATACAAGATAATAGTGACATAAAAG
TAGTGCCAAGAAGAAAAGCAAAGATCATTAGGGATTATGGAAAACAGATGGCAGGTGATGATTGTGTGGC
AAGTAGACAGGATGAGGATTAGAACATGGAAAAGTTTAGTAAAACACCATATGTATGTTTCAGGGAAAGC
TAGGGGATGGTTTTATAGACATCACTATGAAAGCCCTCATCCAAGAATAAGTTCAGAAGTACACATCCCA
CTAGGGGATGCTAGATTGGTAATAACAACATATTGGGGTCTGCATACAGGAGAAAGAGACTGGCATTTGG
GTCAGGGAGTCTCCATAGAATGGAGGAAAAAGAGATATAGCACACAAGTAGACCCTGAACTAGCAGACCA
ACTAATTCATCTGTATTACTTTGACTGTTTTTCAGACTCTGCTATAAGAAAGGCCTTATTAGGACACATA
GTTAGCCCTAGGTGTGAATATCAAGCAGGACATAACAAGGTAGGATCTCTACAATACTTGGCACTAGCAG
CATTAATAACACCAAAAAAGATAAAGCCACCTTTGCCTAGTGTTACGAAACTGACAGAGGATAGATGGAA
CAAGCCCCAGAAGACCAAGGGCCACAGAGGGAGCCACACAATGAATGGACACTAGAGCTTTTAGAGGAGC
TTAAGAATGAAGCTGTTAGACATTTTCCTAGGATTTGGCTCCATGGCTTAGGGCAACATATCTATGAAAC
TTATGGGGATACTTGGGCAGGAGTGGAAGCCATAATAAGAATTCTGCAACAACTGCTGTTTATCCATTTT
CAGAATTGGGTGTCGACATAGCAGAATAGGCGTTACTCGACAGAGGAGAGCAAGAAATGGAGCCAGTAGA
TCCTAGACTAGAGCCCTGGAAGCATCCAGGAAGTCAGCCTAAAACTGCTTGTACCAATTGCTATTGTAAA
AAGTGTTGCTTTCATTGCCAAGTTTGTTTCATAACAAAAGCCTTAGGCATCTCCTATGGCAGGAAGAAGC
GGAGACAGCGACGAAGAGCTCATCAGAACAGTCAGACTCATCAAGCTTCTCTATCAAAGCAGTAAGTAGT
ACATGTAATGCAACCTATACCAATAGTAGCAATAGTAGCATTAGTAGTAGCAATAATAATAGCAATAGTT
GTGTGGTCCATAGTAATCATAGAATATAGGAAAATATTAAGACAAAGAAAAATAGACAGGTTAATTGATA
GACTAATAGAAAGAGCAGAAGACAGTGGCAATGAGAGTGAAGGAGAAATATCAGCACTTGTGGAGATGGG
GGTGGAGATGGGGCACCATGCTCCTTGGGATGTTGATGATCTGTAGTGCTACAGAAAAATTGTGGGTCAC
AGTCTATTATGGGGTACCTGTGTGGAAGGAAGCAACCACCACTCTATTTTGTGCATCAGATGCTAAAGCA
TATGATACAGAGGTACATAATGTTTGGGCCACACATGCCTGTGTACCCACAGACCCCAACCCACAAGAAG
TAGTATTGGTAAATGTGACAGAAAATTTTAACATGTGGAAAAATGACATGGTAGAACAGATGCATGAGGA
TATAATCAGTTTATGGGATCAAAGCCTAAAGCCATGTGTAAAATTAACCCCACTCTGTGTTAGTTTAAAG
TGCACTGATTTGAAGAATGATACTAATACCAATAGTAGTAGCGGGAGAATGATAATGGAGAAAGGAGAGA
TAAAAAACTGCTCTTTCAATATCAGCACAAGCATAAGAGGTAAGGTGCAGAAAGAATATGCATTTTTTTA
TAAACTTGATATAATACCAATAGATAATGATACTACCAGCTATAAGTTGACAAGTTGTAACACCTCAGTC
ATTACACAGGCCTGTCCAAAGGTATCCTTTGAGCCAATTCCCATACATTATTGTGCCCCGGCTGGTTTTG
CGATTCTAAAATGTAATAATAAGACGTTCAATGGAACAGGACCATGTACAAATGTCAGCACAGTACAATG
TACACATGGAATTAGGCCAGTAGTATCAACTCAACTGCTGTTAAATGGCAGTCTAGCAGAAGAAGAGGTA
GTAATTAGATCTGTCAATTTCACGGACAATGCTAAAACCATAATAGTACAGCTGAACACATCTGTAGAAA
TTAATTGTACAAGACCCAACAACAATACAAGAAAAAGAATCCGTATCCAGAGAGGACCAGGGAGAGCATT
TGTTACAATAGGAAAAATAGGAAATATGAGACAAGCACATTGTAACATTAGTAGAGCAAAATGGAATAAC
ACTTTAAAACAGATAGCTAGCAAATTAAGAGAACAATTTGGAAATAATAAAACAATAATCTTTAAGCAAT
CCTCAGGAGGGGACCCAGAAATTGTAACGCACAGTTTTAATTGTGGAGGGGAATTTTTCTACTGTAATTC
AACACAACTGTTTAATAGTACTTGGTTTAATAGTACTTGGAGTACTGAAGGGTCAAATAACACTGAAGGA
AGTGACACAATCACCCTCCCATGCAGAATAAAACAAATTATAAACATGTGGCAGAAAGTAGGAAAAGCAA
TGTATGCCCCTCCCATCAGTGGACAAATTAGATGTTCATCAAATATTACAGGGCTGCTATTAACAAGAGA
TGGTGGTAATAGCAACAATGAGTCCGAGATCTTCAGACCTGGAGGAGGAGATATGAGGGACAATTGGAGA
AGTGAATTATATAAATATAAAGTAGTAAAAATTGAACCATTAGGAGTAGCACCCACCAAGGCAAAGAGAA
GAGTGGTGCAGAGAGAAAAAAGAGCAGTGGGAATAGGAGCTTTGTTCCTTGGGTTCTTGGGAGCAGCAGG
AAGCACTATGGGCGCAGCCTCAATGACGCTGACGGTACAGGCCAGACAATTATTGTCTGGTATAGTGCAG
CAGCAGAACAATTTGCTGAGGGCTATTGAGGCGCAACAGCATCTGTTGCAACTCACAGTCTGGGGCATCA
AGCAGCTCCAGGCAAGAATCCTGGCTGTGGAAAGATACCTAAAGGATCAACAGCTCCTGGGGATTTGGGG
TTGCTCTGGAAAACTCATTTGCACCACTGCTGTGCCTTGGAATGCTAGTTGGAGTAATAAATCTCTGGAA
CAGATTTGGAATCACACGACCTGGATGGAGTGGGACAGAGAAATTAACAATTACACAAGCTTAATACACT
CCTTAATTGAAGAATCGCAAAACCAGCAAGAAAAGAATGAACAAGAATTATTGGAATTAGATAAATGGGC
AAGTTTGTGGAATTGGTTTAACATAACAAATTGGCTGTGGTATATAAAATTATTCATAATGATAGTAGGA
GGCTTGGTAGGTTTAAGAATAGTTTTTGCTGTACTTTCTATAGTGAATAGAGTTAGGCAGGGATATTCAC
CATTATCGTTTCAGACCCACCTCCCAACCCCGAGGGGACCCGACAGGCCCGAAGGAATAGAAGAAGAAGG
TGGAGAGAGAGACAGAGACAGATCCATTCGATTAGTGAACGGATCCTTGGCACTTATCTGGGACGATCTG
CGGAGCCTGTGCCTCTTCAGCTACCACCGCTTGAGAGACTTACTCTTGATTGTAACGAGGATTGTGGAAC
TTCTGGGACGCAGGGGGTGGGAAGCCCTCAAATATTGGTGGAATCTCCTACAGTATTGGAGTCAGGAACT
AAAGAATAGTGCTGTTAGCTTGCTCAATGCCACAGCCATAGCAGTAGCTGAGGGGACAGATAGGGTTATA
GAAGTAGTACAAGGAGCTTGTAGAGCTATTCGCCACATACCTAGAAGAATAAGACAGGGCTTGGAAAGGA
TTTTGCTATAAGATGGGTGGCAAGTGGTCAAAAAGTAGTGTGATTGGATGGCCTACTGTAAGGGAAAGAA
TGAGACGAGCTGAGCCAGCAGCAGATAGGGTGGGAGCAGCATCTCGAGACCTGGAAAAACATGGAGCAAT
CACAAGTAGCAATACAGCAGCTACCAATGCTGCTTGTGCCTGGCTAGAAGCACAAGAGGAGGAGGAGGTG
GGTTTTCCAGTCACACCTCAGGTACCTTTAAGACCAATGACTTACAAGGCAGCTGTAGATCTTAGCCACT
TTTTAAAAGAAAAGGGGGGACTGGAAGGGCTAATTCACTCCCAAAGAAGACAAGATATCCTTGATCTGTG
GATCTACCACACACAAGGCTACTTCCCTGATTAGCAGAACTACACACCAGGGCCAGGGGTCAGATATCCA
CTGACCTTTGGATGGTGCTACAAGCTAGTACCAGTTGAGCCAGATAAGATAGAAGAGGCCAATAAAGGAG
AGAACACCAGCTTGTTACACCCTGTGAGCCTGCATGGGATGGATGACCCGGAGAGAGAAGTGTTAGAGTG
GAGGTTTGACAGCCGCCTAGCATTTCATCACGTGGCCCGAGAGCTGCATCCGGAGTACTTCAAGAACTGC
TGACATCGAGCTTGCTACAAGGGACTTTCCGCTGGGGACTTTCCAGGGAGGCGTGGCCTGGGCGGGACTG
GGGAGTGGCGAGCCCTCAGATCCTGCATATAAGCAGCTGCTTTTTGCCTGTACTGGGTCTCTCTGGTTAG
ACCAGATCTGAGCCTGGGAGCTCTCTGGCTAACTAGGGAACCCACTGCTTAAGCCTCAATAAAGCTTGCC
TTGAGTGCTTC

For quantitative visualization of the detection of gold nanoparticles, specimen were prepared on glass slides for absorption spectral analsysis. A selected single strand oligonucleotide was conjugated with gold nanparticles by adding a mixture containing the desired amount of single strand oligonucleotide to an aqueous nanoparticle suspension or solution containing a nanoparticle precursor. The oligonucleotide functionalized gold nanoparticles are mixed with 0.5% BSA and are deposited onto a glass pate for absorption spectral analysis. Alternatively, the oligonucleotide functionalized gold nanoparticles may be deposited onto a filter paper for hybridization with complementary oligonucleotides sequences to be detected. In the visualization of FIG. 18, both complementary and non-complementary single strand oligonucleotides were pipetted onto the regions of the glass plate where the oligonucleotide functionlized gold nanoparticles were previously deposited, then the glass slide was held at room temperature for 60 seconds before examining using an ultroviolet spectrophotometer for detecting the absorbtion spectra of light passing through the glass slide and the hybridized gold nanoparticles. In FIG. 18 hybridization with complementary DNA (ssDNA-Au+cDNA) is readily distinguishable from either non-complementary DNA (ssDNA-Au+ssDNA) or oligonucleotide functionalized gold nanoparticles only (ssDNA-Au only). This method of testing hybridization on a glass slide is a quick and efficient way of screeing specific complementary oligonucleotide sequences for hybridization with oligonucleotide-functionalized nanoparticles, such as gold.

In one example, a detection kit for viral RNA is prepared using a 33-mer oligonucleotide from the HIV-1 LTR sequence. The LTR sequence is relatively conserved among several HIV-1 strains, including a Glade C HIV 1084i and is not included in any vaccine constructs since none of them are live-attenuated vaccines. Thus, a test positive for this LTR sequence indicates the presence of live HIV-1 strains and does not test positive for antibodies or innoculants used in vaccines. Other gene sequences, such as env, or gag/pol, may be selected to detect viral RNA of HIV-1; however, some of these are included in vaccine constructs, which may cause a positive indication of the genetic probe, due to the presence of the vaccine constructs, especially if being used in vaccine clinical trials for detection of viral loading in a sample of bodily fluid, such as blood, serum, plasma or any other bodily fluid having a sufficient concentration of viral RNA for detection by the test kit. The presence of oligonucleotides used in vaccine constructs may not distinguish HIV-infected individuals from those who have been vaccinated with a candidate vaccine including the oligonucleotides selected for detection within the vaccine constructs. Thus, it is preferred to avoid oligonucleotides used in such vaccine constructs for a test to detect the presence of viral RNA from a live virus.

In one example of a process of preparing a test kit for detection of HIV, a single stranded oligonucleotide complementary to a 33-mer HIV-1 LTR RNA sequence is synthesized and tested for hybridization. First, it may be used to funtionalize carbon nanotubes or gold nanoparticles, such as by thiolating the oligonucleotide. Then, using absorbtion spectral analysis, hybridization with the specific sequence of HIV LTR RNA may be tested. A strong absorbtion spectra shows hybridization of the complementary oligonucleotide, while missing or very weak absorbtion spectra indicate that hybridization failed. One reason for failure is that the oligonucleotide is a non-complementary oligonucleotide. For example, FIGS. 22A and 22B illustrate regions 784, 794 before adding oligonucleotides coupled with single walled carbon nanotubes. Then, a complementary oligonucleotide may be immobilized on the matrix of a cellulose filter membrane, for example, such as the membrane used in an antibody test kit as illustrated in FIGS. 14A-14B. When an HIV-1-infected sample is added to the test cassette, such as by depositing bodily fluids (with or without a diluent), then the complementary oligonucleotide will hybridize with the RNA (HIV-1 LTR) from the infected sample and will become fixed on the matrix of the membrane.

Carbon nanotubes or gold nanoparticles may be functionalized by a second single-stranded oligonucleotide complementary with another region of HIV RNA. If added to the surface of the membrane of the test kit, the functionalized carbon nanotubes or gold nanoparticles hybridize with the HIV RNA, binding the carbon nantobues or gold nanoparticles to a test region on the membrane. When a sufficiently large number of nanotubes or nanoparticles accumulate at a test spot, a contrast between the background and the test spot will become apparent. FIG. 23 illustrates a screening test for determining the concentration of functionalized single wall carbon nanotubes for use in a rapid test kit. For example, a test spot may be visualized during such a test in less than five to ten minutes. Contrast may be enhanced by functionalizing the nanotubes or nanoparticles with a plurality of different complementary oligonucleotide probes targeting different regions of the HIV RNA sequence, such as different regions of the LTR sequence, for example. By limiting the binding of the HIV RNA to the membrane to specific sequences not found in vaccines, no contrast will be apparent, unless the virus is present in the bodily fluid tested. Nevertheless, one HIV RNA strand or sequence may bind multiple functionalized nanotubes or nanoparticles, if such nanotubes or nanoparticles are functionalized with a plurality of complementary oligonucleotides, improving contrast. In this way, the signal (i.e. contrast) is amplified without the need to amplify the concentration of RNA, such as by using a PCR technique, but only an HIV-infected sample will give a positive result. Therefore, the positive result in the viral RNA test will indicate a true HIV-1 infection, and samples collected from those individuals who are vaccinated with any candidate vaccine constructs will be negative in the viral RNA test.

In one example, the complementary oligonucleotides used for functionalizing a nanotube or nanoparticle are thiolated at the 5′-end and are mixed with gold nanoparticles. The complementary oligonucleotides may be complementary to a region of the HIV virus, such as the LTR region HIV RNA, for example. In one example, different types of nanoparticles or nanoparticles functionalized with different markers are added to specific complementary oligonucleotides. Then, the presence of a concentration of the specific type of nanoparticles or the markers associated with specific nanoparticles will indicate the presence of one type of complimentary oligonucleotide. Since one complimentary oligonucleotide may be selected to hybridize with a specific type or strain of HIV, a test kit is able to detect specific types and strains of HIV RNA present in a sample of a bodily fluid, for example. In one example, complementary oligonucleotides are selected that are common to many different strains of HIV and/or to both HIV-1 and HIV-2. Several different complementary oligonucleotides may be immobilized on a membrane, such as a cellulose filter paper, in one or more than one area, and each of the plurality of complementary oligonucleotides may be selected to immobilize one or more different HIV types or strains.

In one example, a protocol described in Glynow, K et al., Oligonucleotide-functionalized gold nanoparticles as probes in a dry-reagent strip biosensor for DNA analysis by hybridization, Anal. Chem., 75(16), (2003) p. 4155-60 is used to prepare a functionalized genetic probe using gold nanoparticles added to a stain used in an HIV antibody test kit. The test kit includes a single window, as illustrated in FIG. 14B, for example, having test spots for detecting the presence of both antibodies and HIV RNA. For example, the oligonucleotide probe may be synthesized and thiolated. Then, 0.9 nmol of the thiolated DNA probe is added to a 10 μl suspension of gold nanoparticles (about 1.5 pmol) at 4° C. for 24 hrs. A sodium chloride solution is added to the mixture to a concentration of 90 nmol/L, and is allowed to stand for another 24 hrs at 4° C. The oligonucleotide-functionalized gold nanoparticles may be centrifuged at 2800 g for 45 min and may be suspended in 600 μl of 30% sucrose with 45 nmol/L NaCl. In one example, the functionalized gold nanoparticle probes are lyophilized and stored at room temperature for prolonged periods.

In one method for detection of viral RNA, 50 μl of an HIV-infected blood is diluted in 150 μl hybridization buffer. The sample may be added to a preassembled test cassette having an LTR-complementary oligonucleotide immobilized on a test spot. After the blood sample is absorbed, 200 μl of the functionalized gold nanoparticles are added to the test well as a genetic probe. The genetic prove hybridizes the gold nanoparticles to complementary regions of the viral RNA. Then, 200 μl of washing buffer, such as a saline solution, is added to reduce the background color and to increase contrast between the background and the test spot. The entire viral RNA test can be performed in less than 5 to 10 minutes. Color of a test kit may remain stable for 48 hrs at room temperature.

### Optimization of the ULTraPID-RNA (Au) Platform

In one example, a 33-nt oligonucleotide from HIV 89.6 proviral clone is immobilized on the membrane test spot of an HIV test kit. This immobilized DNA primer is capable of hybridizing and fixing HIV LTR RNA on the test spot, because it is complementary to the HIV LTR region, including a Glade C HIV 1084i. Other DNA sequences complementary to the LTR may be used, alternatively or in addition to this oligonucleotide. In one example, the length of the oligonucleotide is optimized for efficiency in capturing HIV RNA having a complementary LTR region.

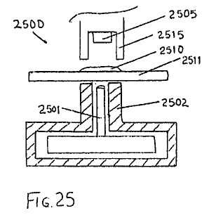

In one example, a fluorescent oligonucleotide (siGLO from Thermo Scientific Dharmacon, Colo.) was added directly to the membrane of the ULTraPID cassette, and the binding was insufficient to prevent the fluorescent marker from being washed off the membrane surface with 0.3 M NaCl, which is normally included in the gold nanoparticle staining buffer. Thus, direct application of the complementary oligonucleotide to the surface of the member is not adequate for immobilizing the oligonucleotide. In another example, a fluorescent oligonucleotide was first conjugated with chitosan nanoparticles, and the conjugate was applied to the surface of the membrane. FIG. 24 illustrates that the conjugate was stably immobilized on the membrane in this example. FIG. 24 has a green fluorescence 607 of the fluorescent oligonucleotide (converted to white in this image for purposes of illustration only), which contrasts with the uniformly dark (no fluorescence) background 606 of a sample prepared without first conjugating the fluorescent oligonucleotide with chitosan nanoparticles. The fluorescent oligonucleotide is rinsed from the cellulose filter paper in the background 606 image, while the oligonucleotide conjugated with chitosan and chitosan derivatives immobilizes the fluorescent oligonucleotide on a portion of the cellulose filter paper. It is believed that the chitosan forms a cationic polymer that binds to the matrix of the membrane when conjugated with an oligonucleotide. Thus, the chitosan helps to immobilize the complimentary oligonucleotides to the membrane used for testing for viral RNA or other RNA or DNA to be detected. With adequate binding to the membrane and immobilization of the target sequence of the RNA or DNA, the one or more complementary oligonucleotides functionalizing nanotubes or nanoparticles may be hybridized on the target RNA or DNA to be detetected, providing contrast between the genetic probe test spots (G) and the background of the cellulose filter paper. Then, the genetic probe may be used to detect the presence of a specific RNA or DNA sequence that binds to the complementary oligonucleotides immobilized on the filter paper and coupled with the nanotubes and/or nanoparticles, and may be used to distinguish viral DNA from the mere presence of antibodies for viral DNA, for example.

In other examples, bovine serum albumin (BSA) or streptavidin are used either alone or in combination with conjugation with chitosan nanoparticles to enhance the binding of the 33-nt oligonucleotide on a cellulose filter paper membrane, such as the membranes used in the examples of antibody test kits. In yet another example, a portion of the membrane is coated with streptavidin, and a biotinylated 33-nt oligonucleotide is applied to the streptavidin coated portion of the membrane.

In FIG. 25 a schematic of a detector 2500 is sketched that is capable of measuring light emitted by a detection region 2510 or transmitted through a detection region 2510 of a slide 2511. A light source 2501 is provided in a housing 2502, and a charge coupled device or other photodetector or spectral analyzer 2505 is provided with a shield 2515 or collimator to capture and analyze the light. Filters and optics may be provided as is known in the art for such detectors.

Alternative combinations and variations of the examples provided will become apparent based on this disclosure. It is not possible to provide specific examples for all of the many possible combinations and variations of the embodiments described, but such combinations and variations may be claims that eventually issue.

Table 1A shows measurements of flow rate for a given particle retention size:

| Particle Retention Size | Flow Rate of DPBS | Flow Rate of Water |
|---|---|---|
| (μm) | (mL/min/cm²) | (mL/min/cm²) |
| 2.5 | 0.045 | 0.040 |
| 6 | 0.114 | 0.128 |
| 11 | 0.162 | 0.130 |
| 20-25 | 0.165 | 0.214 |
| 23 | 0.358 | 0.406 |
| 30 | 1.218 | 0.641 |

Table 1B shows flow rate data for nitrocellulose mixed ester membranes with various pore sizes.

Typical Characteristics

Flowrates

| Pore Size | Water | Air | Bubble Point |
|---|---|---|---|
| 0.10 μm | 6.5 | 0.8 | 100 psi |
| 0.22 μm | 18.5 | 2 | 50 psi |
| 0.45 μm | 50 | 4 | 30 psi |
| 0.65 μm | 125 | 9 | 17 psi |
| 0.80 μm | 195 | 17 | 15 psi |
| 1.2 μm | 290 | 20 | 12 psi |
| 5.0 μm | 550 | 34 | 7 psi |

TABLE 1C
Typical Properties of Cellulose Filters

| | | | | | | | | |
|---|---|---|---|---|---|---|---|---|
| | Particle | | | Typical | Basis | | | Tensile |
| | Retention* | Air Flow Rate | | Thickness | Weight | Wet Burst | Dry Burst | M/D Dry |
| Grade | Liquid (μm) | (s/100 mL/in²) | Ash (%) | (μm) | (g/m²) | (psi) | (psi) | (N/15 mm) |

Qualitative

| | | | | | | | | |
|---|---|---|---|---|---|---|---|---|
| 1 | 11 | 10.5 | 0.06 | 180 | 88 | 0.3 | 16 | 39.1 |
| 2 | 8 | 21 | 0.06 | 190 | 103 | 0.7 | 16 | 44.6 |
| 3 | 6 | 26 | 0.06 | 390 | 187 | 0.5 | 28 | 72 |
| 4 | 20-25 | 3.7 | 0.06 | 205 | 96 | 0.7 | 10 | 28.4 |
| 5 | 2.5 | 94 | 0.06 | 200 | 98 | 0.4 | 21 | 55.6 |
| 6 | 3 | 35 | 0.2 | 180 | 105 | 0.3 | 15 | 39.1 |

General Purpose and Wet Strengthened Qualitative

| | | | | | | | | |
|---|---|---|---|---|---|---|---|---|
| 91 | 10 | 6.2 | N/A | 205 | 71 | 2 | 18 | 28 |
| 93 | 10 | 7 | N/A | 145 | 67 | 2.6 | 12 | 38 |
| 113 | 30 | 1.3 | N/A | 420 | 131 | 8 | 24 | 38.6 |
| 114 | 23 | 5.3 | N/A | 190 | 77 | 8.9 | 15 | 42.1 |

TABLE 2

| Low Titer | | | | | | | | | |
|---|---|---|---|---|---|---|---|---|---|
| DPBS Flow | Particle | | | | | | | | |
| Rate | Retention | Test | | | | Control | | | |
| (mL/min/.cm²) | Size (μm) | 1 | 2 | 3 | 4 | 1 | 2 | 3 | 4 |
| 0.045 | 2.5 | 1 | 2 | 1 | 1 | 4 | 4 | 3 | 2 |
| 0.114 | 6 | 1 | 1 | 1 | 1 | 3 | 4 | 3 | 3 |
| 0.162 | 11 | 1 | 1 | 1 | 1 | 3 | 3 | 2 | 2 |
| 0.165 | 20-25 | 1 | 1 | 1 | 1 | 2 | 3 | 2 | 2 |
| 0.358 | 23 | 0 | 0 | 1 | 1 | 1 | 1 | 2 | 2 |
| 1.218 | 30 | 0 | 0 | 0 | 0 | 1 | 1 | 0 | 1 |
| High Titer | | | | | | | | | |
| DPBS Flow | Particle | | | | | | | | |
| Rate | Retention | Test | | | | Control | | | |
| (mL/min./cm²) | Size (μm) | 1 | 2 | 3 | 4 | 1 | 2 | 3 | 4 |
| 0.045 | 2.5 | 4 | 4 | 3 | 2 | 4 | 4 | 4 | 4 |
| 0.114 | 6 | 3 | 4 | 3 | 3 | 4 | 4 | 4 | 4 |
| 0.162 | 11 | 3 | 3 | 2 | 2 | 4 | 4 | 4 | 4 |
| 0.165 | 20-25 | 2 | 3 | 2 | 2 | 4 | 3 | 4 | 4 |
| 0.358 | 23 | 1 | 1 | 2 | 2 | 4 | 4 | 4 | 4 |
| 1.218 | 30 | 1 | 1 | 0 | 1 | 1 | 1 | 1 | 1 |
| HIV Negative | | | | | | | | | |
| DPBS Flow | Particle | | | | | | | | |
| Rate | Retention | Test | | | | Control | | | |
| (mL/min./cm²) | Size (μm) | 1 | 2 | 3 | 4 | 1 | 2 | 3 | 4 |
| 0.114 | 6 | 0 | 0 | 0 | 0 | 4 | 4 | 4 | 4 |

TABLE 3

| | BLOOD | | | | PLASMA | | | |
|---|---|---|---|---|---|---|---|---|
| | Trial 1 | | Trial 2 | | Trial 1 | | Trial 2 | |
| Sample Number | C | T | C | T | C | T | C | T |
| 80 | 4 | 1 | 4 | 1 | 4 | 2 | 4 | 2 |
| 81 | 4 | 2 | 4 | 1 | 4 | 2 | 4 | 2 |
| 82 | 4 | 3 | 4 | 4 | 4 | 4 | 4 | 4 |
| 83 | 4 | 3 | 4 | 3 | 4 | 3 | 4 | 3 |
| 84 | 4 | 3 | 4 | 3 | 4 | 3 | 4 | 3 |
| 85 | 4 | 3 | 4 | 3 | 4 | 4 | 4 | 4 |
| 86 | 4 | 2 | — | — | 4 | 2 | — | — |
| 87 | 4 | 4 | — | — | 4 | 4 | — | — |
| 88 | 4 | 3 | — | — | 4 | 4 | — | — |
| 89 | 4 | 3 | — | — | 4 | 3 | — | — |
| 90 | 4 | 4 | — | — | 4 | 3 | — | — |
| 91 | 4 | 1 | — | — | 4 | 1 | — | — |
| 92 | 4 | 3 | — | — | 4 | 3 | — | — |
| 93 | 4 | 4 | — | — | 4 | 4 | — | — |
| 94 | 4 | 3 | — | — | 4 | 3 | — | — |
| 95 | 4 | 2 | — | — | 4 | 2 | — | — |
| 96 | 4 | 4 | — | — | 4 | 4 | — | — |

TABLE 4
Assay comparison for commercial kits and example test kits.

| | | | | | | Trinity | Example Test | |
|---|---|---|---|---|---|---|---|---|
| I.D. # | Western | Abbott | Murex | OraSure | MedMira | Uni- | Kits | |
| PRB204 | Blot | Determine ™ | SUDS | OraQuick ® | Reveal ® | Gold ™ | Result | CIV |
| 01 | − | − | − | − | − | − | No | No |
| | | | | | | | test | test |
| 02 | + | + | + | + | + | + | + | 4 |
| 03 | − | − | − | − | − | − | No | No |
| | | | | | | | test | test |
| 04 | + | + | + | + | + | + | + | 3 |
| 05 | + | + | + | + | + | + | + | 4 |
| 06 | + | + | + | + | + | + | + | 4 |
| 07 | + | + | + | + | + | + | + | 4 |
| 08 | + | + | + | + | + | + | + | 3 |
| 09 | − | − | − | − | − | − | No | No |
| | | | | | | | test | test |
| 010 | Ind. | + | − | − | − | − | + | 1 |
| 011 | + | + | + | + | + | + | + | 4 |
| 012 | + | + | + | + | + | + | + | 4 |
| 013 | Ind. | + | + | +/− | + | − | + | 2 |
| 014 | + | + | + | + | + | + | + | 4 |
| 015 | + | + | + | + | + | + | + | 4 |
| 016 | + | + | + | + | + | + | + | 3 |
| 017 | + | + | + | + | + | + | + | 3 |
| 018 | Ind. | + | − | +/− | + | − | + | 2 |
| 019 | + | + | + | + | + | − | + | 2 |
| 020 | + | + | + | + | + | + | + | 4 |
| 021 | + | + | + | + | + | − | + | 4 |
| 022 | + | + | + | + | + | − | + | 4 |
| 023 | − | − | − | − | − | − | No | No |
| | | | | | | | test | test |
| 024 | Ind. | + | − | − | − | − | − | 0 |
| 025 | Ind. | + | − | + | − | + | − | 0 |

TABLE 5
Assay comparison for commercial kits and example test kits
(band patterns)

| I.D. # PRB204 | RL37 - Band Pattern |
|---|---|
| 01 | No bands |
| 02 | 18, 24, 31, 41, 55, 51, 65, 120, 160 |
| 03 | No bands |
| 04 | 18, 24, 31, 41, 55, 51, 65, 120, 160 |
| 05 | 24, 31, 41, 51, 55, 65, 120, 160 |
| 06 | 18, 24, 31, 41, 55, 51, 65, 120, 160 |
| 07 | 18, 24, 31, 41, 55, 51, 65, 120, 160 |
| 08 | f18, 24, 31, 41, 51, 55, 120, 160 |
| 09 | No bands |
| 10 | 24, 51, 55, f160 |
| 11 | 18, 24, 31, 41, 55, 51, 65, 120, 160 |
| 12 | 18, 24, 31, 41, 55, 51, 65, 120, 160 |
| 13 | 24, f51, f55, f160 |
| 14 | 18, 24, 31, 41, 55, 51, 65, 120, 160 |
| 15 | 18, 24, 31, 41, 55, 51, 65, 120, 160 |
| 16 | 24, f41, 51, 55, 160 |
| 17 | 24, 51, 55, f120, 160 |
| 18 | f24, f51, f55, 160 |
| 19 | 24, f31, 51, 55, f120, 160 |
| 20 | 18, 24, 31, 41, 51, 55, 65, 120, 160 |
| 21 | 24, 51, 55, 160 |
| 22 | 24, 31, 41, 51, 55, 65, 120, 160 |
| 23 | No bands |
| 24 | 24, f51, f55, f160 |
| 25 | f51, f55 |

TABLE 6

| Whatman Grade | GF/C | |
|---|---|---|
| Particle Retention | 1.2 | μm |
| Flowrate | 10.5 | sec./100 mL |
| Thickness | 0.26 | mm |
| Weight | 53 | g/m² |
| Max. Temp. | 500° | C. (932° F.) |
| Water Absorption | 250 | mL/m² |

| | |
|---|---|
| Sterilization | Autoclavable |

TABLE 7

| Sample Number | Test Kit | MedMira Reveal ® G3 |
|---|---|---|
| 80 | 2 | 1 |
| 81 | 2 | 3 |
| 82 | 4 | 2 |
| 83 | 3 | 2 |
| 84 | 3 | 2 |
| 85 | 4 | 3 |
| 86 | 2 | 2 |
| 91 | 1 | 1 |

TABLE 8A

| | Draft Re- | | Logical | | Shared HIV | | Unique HIV | | |
|---|---|---|---|---|---|---|---|---|---|
| | arrangement | | Approach | | Regions | | Regions | | # nucleotides |
| COMPARISON | 45 | 62 | 45 | 62 | 45 | 62 | 1 | 44 | 44 |
| WITH HIV | | | | | | | | | |
| NC_001802 | | | | | | | | | |

HUMAN CHROMOSOME

| | | | | | | | | | | | |
|---|---|---|---|---|---|---|---|---|---|---|---|
| 409 | 447 | 2 | 182 | 209 | 182 | 209 | 232 | 251 | 213 | 231 | 19 |
| 5745 | 5774 | 11 | 196 | 212 | 182 | 212 | 365 | 376 | 252 | 364 | 113 |
| 5797 | 5848 | 5 | 232 | 251 | 232 | 251 | 388 | 447 | 377 | 387 | 11 |
| 7272 | 7301 | 4 | 365 | 376 | 365 | 376 | 540 | 587 | 448 | 539 | 92 |
| 407 | 436 | 4 | 388 | 420 | 388 | 420 | 617 | 628 | 588 | 616 | 29 |
| 6752 | 6791 | 2 | 407 | 420 | 388 | 420 | 637 | 711 | 629 | 636 | 8 |
| 7201 | 7230 | 11 | 407 | 425 | 407 | 425 | 736 | 761 | 712 | 735 | 24 |
| 5797 | 5848 | 5 | 408 | 436 | 407 | 436 | 930 | 950 | 762 | 929 | 168 |
| 3277 | 3308 | 3 | 409 | 439 | 408 | 439 | 1114 | 1134 | 951 | 1113 | 163 |
| 5902 | 5930 | 7 | 410 | 440 | 409 | 440 | 1142 | 1158 | 1135 | 1141 | 7 |
| 2479 | 2553 | 6 | 410 | 447 | 410 | 447 | 1180 | 1196 | 1159 | 1179 | 21 |
| 6293 | 6335 | 3 | 414 | 447 | 410 | 447 | 1256 | 1270 | 1197 | 1255 | 59 |
| 3365 | 3388 | 11 | 540 | 578 | 540 | 578 | 1277 | 1289 | 1271 | 1276 | 6 |
| 5669 | 5715 | 7 | 575 | 587 | 540 | 587 | 1293 | 1304 | 1290 | 1292 | 3 |
| 4225 | 4258 | 1 | 617 | 628 | 617 | 628 | 1326 | 1337 | 1305 | 1325 | 21 |
| 9002 | 9040 | X | 637 | 662 | 637 | 662 | 1358 | 1377 | 1338 | 1357 | 20 |
| 647 | 680 | X | 642 | 680 | 637 | 680 | 1423 | 1438 | 1378 | 1422 | 45 |
| 2479 | 2553 | 6 | 647 | 682 | 642 | 682 | 1464 | 1483 | 1439 | 1463 | 25 |
| 7937 | 7960 | 3 | 647 | 689 | 647 | 689 | 1491 | 1534 | 1484 | 1490 | 7 |
| 3277 | 3308 | 3 | 678 | 695 | 647 | 695 | 1546 | 1623 | 1535 | 1545 | 11 |

TABLE 8B

| | | | Draft Re- | | Logical | | Shared HIV | | Unique HIV | | |
|---|---|---|---|---|---|---|---|---|---|---|---|
| MOUSE CHROMOSOME | | | arrangement | | Approach | | Regions | | Regions | | # nucleotides |
| 2257 | 2342 | 2, 7, 8, 10, 12, 14, | 685 | 701 | 683 | 701 | 1668 | 1686 | 1660 | 1667 | 8 |
| | | 15, 16, X | | | | | | | | | |
| 7202 | 7242 | 14 | 700 | 711 | 685 | 711 | 1702 | 1730 | 1687 | 1701 | 15 |
| 3649 | 3682 | 10 | 736 | 761 | 736 | 761 | 1756 | 2828 | 1731 | 1755 | 25 |
| 6659 | 6691 | 10 | 930 | 941 | 930 | 941 | 2861 | 2872 | 2829 | 2860 | 32 |
| 182 | 212 | 10 | 930 | 950 | 930 | 950 | 2931 | 2944 | 2873 | 2930 | 58 |
| 6282 | 6368 | 5, 15 | 1114 | 1125 | 1114 | 1125 | 2954 | 6021 | 2945 | 2953 | 9 |
| 2985 | 3017 | 15 | 1119 | 1134 | 1114 | 1134 | 6033 | 6117 | 6022 | 6032 | 11 |
| 3910 | 3938 | 2 | 1142 | 1155 | 1142 | 1155 | 6124 | 6137 | 6118 | 6123 | 6 |
| 4517 | 4571 | 3, 8 | 1145 | 1158 | 1142 | 1158 | 6153 | 6197 | 6138 | 6152 | 15 |
| 7210 | 7242 | 1 | 1180 | 1196 | 1180 | 1196 | 6198 | 6237 | 6198 | 6197 | 0 |
| 3968 | 3994 | X | 1256 | 1270 | 1256 | 1270 | 6247 | 6267 | 6238 | 6246 | 9 |
| 5689 | 5748 | 3 | 1277 | 1289 | 1277 | 1289 | 6272 | 6368 | 6268 | 6271 | 4 |
| 6169 | 6197 | 2 | 1293 | 1304 | 1293 | 1304 | 6515 | 6534 | 6369 | 6514 | 146 |
| 1702 | 1730 | 14 | 1326 | 1337 | 1326 | 1337 | 6542 | 6556 | 6535 | 6541 | 7 |
| 1584 | 1614 | 11 | 1358 | 1377 | 1358 | 1377 | 6659 | 6691 | 6557 | 6658 | 102 |
| 5689 | 5825 | 3, 16 | 1423 | 1438 | 1423 | 1438 | 6717 | 6791 | 6692 | 6716 | 25 |
| 4895 | 4928 | 16 | 1464 | 1483 | 1464 | 1483 | 6795 | 6809 | 6792 | 6794 | 3 |
| 8516 | 8574 | 5, 12 | 1491 | 1502 | 1491 | 1502 | 6815 | 6855 | 6810 | 6814 | 5 |
| 1771 | 1797 | 12 | 1493 | 1515 | 1491 | 1515 | 6865 | 6876 | 6856 | 6864 | 9 |
| 7274 | 7330 | 17 | 1502 | 1534 | 1493 | 1534 | 6895 | 6912 | 6877 | 6894 | 18 |
| 4517 | 4542 | 3 | 1502 | 1534 | 1502 | 1534 | 6942 | 6968 | 6913 | 6941 | 29 |
| 7733 | 7762 | 13 | 1546 | 1564 | 1546 | 1564 | 7053 | 7101 | 6969 | 7052 | 84 |
| 4192 | 4270 | 4, 6 | 1557 | 1570 | 1546 | 1570 | 7108 | 7121 | 7102 | 7107 | 6 |
| 410 | 440 | 6 | 1561 | 1603 | 1557 | 1603 | 7169 | 7190 | 7122 | 7168 | 47 |
| 3019 | 3050 | 13 | 1574 | 1609 | 1561 | 1609 | 7197 | 7247 | 7191 | 7196 | 6 |
| 4234 | 4270 | 4 | 1584 | 1614 | 1574 | 1614 | 7254 | 7350 | 7248 | 7253 | 6 |
| 6722 | 6764 | | 1610 | 1623 | 1584 | 1623 | 7454 | 7474 | 7351 | 7453 | 103 |
| 3649 | 3685 | 10 | 1632 | 1656 | 1632 | 1656 | 7486 | 7501 | 7475 | 7485 | 11 |

TABLE 8B

| MOUSE | | | Draft Re- | | Logical | | Shared HIV | | Unique HIV | | |
|---|---|---|---|---|---|---|---|---|---|---|---|
| CHROMOSOME | | | arrangement | | Approach | | Regions | | Regions | | # nucleotides |
| 8389 | 8421 | 4 | 1642 | 1659 | 1632 | 1659 | 7559 | 7578 | 7502 | 7558 | 57 |
| 6045 | 6117 | 3, 11 | 1668 | 1686 | 1668 | 1686 | 7582 | 7600 | 7579 | 7581 | 3 |
| 7814 | 7836 | 9 | 1702 | 1729 | 1702 | 1729 | 7717 | 7766 | 7601 | 7716 | 116 |
| 6282 | 6368 | 5 | 1702 | 1730 | 1702 | 1730 | 7814 | 7836 | 7767 | 7813 | 47 |
| 8547 | 8574 | 5 | 1756 | 1773 | 1756 | 1773 | 7855 | 7872 | 7837 | 7854 | 18 |
| 7074 | 7101 | 4 | 1757 | 1784 | 1756 | 1784 | 7901 | 7915 | 7873 | 7900 | 28 |
| 5112 | 5137 | 1 | 1763 | 1786 | 1757 | 1786 | 7928 | 7960 | 7916 | 7927 | 12 |
| 4236 | 4261 | 3 | 1771 | 1790 | 1763 | 1790 | 8110 | 8121 | 7961 | 8109 | 149 |

TABLE 8C

| | | | Draft Re- | | Logical | | Shared HIV | | Unique HIV | | |
|---|---|---|---|---|---|---|---|---|---|---|---|
| Rat Chromosome | | | arrangement | | Approach | | Regions | | Regions | | # nucleotides |
| 5606 | 5823 | Rat Chr 1, 15, 18, | 1777 | 1842 | 1772 | 1842 | 8188 | 8202 | 8157 | 8187 | 31 |
| | | 19 | | | | | | | | | |
| 1702 | 1729 | Rat Chr 18 | 1825 | 1884 | 1777 | 1884 | 8334 | 8363 | 8203 | 8333 | 131 |
| 4193 | 4339 | Rat Chr 3, 5, 8, 10, | 1859 | 1887 | 1825 | 1887 | 8377 | 8421 | 8364 | 8376 | 13 |
| | | 11, 12, 13, 14, 15, | | | | | | | | | |
| | | 16, 17, X | | | | | | | | | |
| 7254 | 7313 | Rat Chr 5, 15 | 1873 | 1932 | 1859 | 1932 | 8449 | 8463 | 8422 | 8448 | 27 |
| 4060 | 4095 | Rat Chr 7 | 1902 | 1963 | 1873 | 1963 | 8516 | 8673 | 8464 | 8515 | 52 |
| 3277 | 3317 | Rat Chr 2 | 1945 | 2056 | 1902 | 2056 | 8684 | 8715 | 8674 | 8683 | 10 |
| 1502 | 1534 | Rat Chr 7 | 2035 | 2173 | 1945 | 2173 | 8745 | 8758 | 8716 | 8744 | 29 |
| 180 | 209 | Rat Chr 12, 20 | 2126 | 2184 | 2035 | 2184 | 8785 | 8804 | 8759 | 8784 | 26 |
| 1561 | 1603 | Rat Chr X | 2148 | 2186 | 2126 | 2186 | 8864 | 8879 | 8805 | 8863 | 59 |
| 5032 | 5066 | Rat Chr 6 | 2156 | 2197 | 2148 | 2197 | 9002 | 9040 | 8880 | 9001 | 122 |
| 2156 | 2198 | Rat Chr 5, X | 2170 | 2197 | 2156 | 2197 | 9057 | 9076 | 9041 | 9056 | 16 |
| 6033 | 6068 | Rat Chr 3 | 2171 | 2198 | 2170 | 2198 | 9130 | 9147 | 9077 | 9129 | 53 |
| 3573 | 3602 | Rat Chr 2 | 2178 | 2203 | 2171 | 2203 | | | | | |
| 2264 | 2342 | Rat Chr 1 | 2182 | 2220 | 2178 | 2220 | | | | | |
| 410 | 439 | Rat Chr 4, X | 2186 | 2239 | 2182 | 2239 | | | | | |
| 6272 | 6314 | Rat Chr 9 | 2192 | 2243 | 2186 | 2243 | | | | | |
| 8684 | 8715 | Rat Chr 7 | 2207 | 2243 | 2192 | 2243 | | | | | |
| 2224 | 2311 | Rat Chr 7 | 2224 | 2300 | 2207 | 2300 | | | | | |
| 2587 | 2620 | Rat Chr 4 | 2228 | 2311 | 2224 | 2311 | | | | | |
| 4724 | 4751 | Rat Chr 10 | 2231 | 2314 | 2228 | 2314 | | | | | |
| 3762 | 3797 | Rat Chr 10, 18, 19 | 2231 | 2322 | 2231 | 2322 | | | | | |
| 4289 | 4339 | Rat Chr 7 | 2257 | 2338 | 2231 | 2338 | | | | | |
| 2587 | 2620 | Rat Chr 4 | 2264 | 2342 | 2257 | 2342 | | | | | |
| 642 | 689 | Rat Chr 15 | 2285 | 2342 | 2264 | 2342 | | | | | |
| 7202 | 7229 | Rat Chr 15 | 2308 | 2468 | 2285 | 2468 | | | | | |
| 6198 | 6233 | Rat Chr 11 | 2312 | 2550 | 2308 | 2550 | | | | | |

TABLE 8C

| | | | | | |
|---|---|---|---|---|---|
| | Draft | Logical | Shared | Unique | |
| Rat Chromosome | Rearrangement | Approach | HIV Regions | HIV Regions | # nucleotides |

| | | | | | | |
|---|---|---|---|---|---|---|
| 2954 | 2994 | Rat Chr 10 | 2456 | 2553 | 2312 | 2553 |
| 3971 | 4016 | Rat Chr 9 | 2478 | 2553 | 2456 | 2553 |
| 1502 | 1534 | Rat Chr 7 | 2479 | 2553 | 2478 | 2553 |
| 8527 | 8564 | Rat Chr 5 | 2479 | 2560 | 2479 | 2560 |
| 6748 | 6787 | Rat Chr 4 | 2513 | 2571 | 2479 | 2571 |
| 1632 | 1659 | Rat Chr 4 | 2548 | 2598 | 2513 | 2598 |
| 6828 | 6855 | Rat Chr 4 | 2559 | 2615 | 2548 | 2615 |

TABLE 8D

| | | | | | |
|---|---|---|---|---|---|
| | Draft | Logical | | | |
| Chimp Chromosome | Rearrangement | Approach | Shared HIV Regions | Unique HIV Regions | # nucleotides |

| | | | | | | |
|---|---|---|---|---|---|---|
| 4800 | 4838 | Chimp Chr 13 | 2587 | 2620 | 2571 | 2620 |
| 5745 | 5822 | Chimp Chr 11, X | 2587 | 2620 | 2587 | 2620 |
| 7265 | 7306 | Chimp Chr 4, 11 | 2599 | 2621 | 2587 | 2621 |
| 407 | 447 | Chimp Chr 2B, 3, 4, | 2607 | 2632 | 2599 | 2632 |
| | | 13 | | | | |
| 637 | 682 | Chimp Chr 4 | 2609 | 2652 | 2607 | 2652 |
| 3277 | 3388 | Chimp Chr 3, 11 | 2623 | 2652 | 2609 | 2652 |
| 2478 | 2553 | Chimp Chr 2A, 6, 8 | 2638 | 2653 | 2623 | 2653 |
| 6815 | 6855 | Chimp Chr 5 | 2639 | 2655 | 2638 | 2655 |
| 7937 | 7960 | Chimp Chr 3 | 2643 | 2727 | 2639 | 2727 |
| 4174 | 4274 | Chimp Chr 1, 2B, 5, | 2715 | 2733 | 2643 | 2733 |
| | | 6, 7, 17, 19 | | | | |
| 6663 | 6689 | Chimp Chr 1 | 2716 | 2760 | 2715 | 2760 |
| 7720 | 7747 | Chimp Chr X | 2746 | 2773 | 2716 | 2773 |
| 7197 | 7247 | Chimp Chr 10, X | 2754 | 2776 | 2746 | 2776 |
| 7263 | 7303 | Chimp Chr 18 | 2756 | 2797 | 2754 | 2797 |
| 2148 | 2173 | Chimp Chr 18 | 2777 | 2827 | 2756 | 2827 |
| 3116 | 3167 | Chimp Chr 6, 9, 18 | 2816 | 2828 | 2777 | 2828 |
| 3514 | 3539 | Chimp Chr 18 | 2861 | 2872 | 2861 | 2872 |
| 1902 | 1932 | Chimp Chr 15 | 2931 | 2944 | 2931 | 2944 |
| 7315 | 7350 | Chimp Chr 14 | 2954 | 2980 | 2954 | 2980 |
| 2607 | 2632 | Chimp Chr 11, 14 | 2969 | 2986 | 2954 | 2986 |
| 6033 | 6063 | Chimp Chr 11 | 2975 | 2994 | 2969 | 2994 |
| 3724 | 3749 | Chimp Chr 10 | 2981 | 2995 | 2975 | 2995 |
| 4637 | 4664 | Chimp Chr 9 | 2985 | 3014 | 2981 | 3014 |
| 2623 | 2653 | Chimp Chr 8 | 3000 | 3017 | 2985 | 3017 |
| 3995 | 4027 | Chimp Chr 7 | 3006 | 3050 | 3000 | 3050 |
| 5902 | 5929 | Chimp Chr 7 | 3019 | 3081 | 3006 | 3081 |
| 6734 | 6766 | Chimp Chr 5 | 3070 | 3103 | 3019 | 3103 |
| 8516 | 8543 | Chimp Chr 4 | 3091 | 3115 | 3070 | 3115 |
| 540 | 578 | Chimp Chr 4 | 3101 | 3122 | 3091 | 3122 |
| 5701 | 5726 | Chimp Chr 2B | 3105 | 3144 | 3101 | 3144 |
| 1574 | 1609 | Chimp Chr 1 | 3116 | 3153 | 3105 | 3153 |

TABLE 8E

| | | | | | |
|---|---|---|---|---|---|
| | Draft | Logical | | | |
| VIRUS | Rearrangement | Approach | Shared HIV Regions | Unique HIV Regions | # nucleotides |

| | | | | | | |
|---|---|---|---|---|---|---|
| | | | 3135 | 3161 | 3132 | 3161 |
| 1859 | 1884 | HTLV-1 | 3139 | 3167 | 3135 | 3167 |
| 6302 | 6321 | HTLV-1 | 3145 | 3223 | 3139 | 3223 |
| 5815 | 5837 | HTLV-1 | 3206 | 3289 | 3145 | 3289 |
| 1763 | 1786 | HTLV-1 | 3244 | 3308 | 3206 | 3308 |
| 408 | 420 | HTLV-1 | 3277 | 3308 | 3244 | 3308 |
| 7928 | 7940 | HTLV-1 | 3277 | 3317 | 3277 | 3317 |
| 6124 | 6135 | HTLV-1 | 3277 | 3363 | 3277 | 3363 |
| 6660 | 6671 | HTLV-1 | 3277 | 3370 | 3277 | 3370 |
| 8188 | 8202 | HTLV-1 | 3277 | 3388 | 3277 | 3388 |
| 2638 | 2652 | HTLV-1 | 3358 | 3388 | 3277 | 3388 |
| 3139 | 3153 | HTLV-1 | 3365 | 3526 | 3358 | 3526 |
| 4478 | 4489 | HTLV-1 | 3505 | 3539 | 3365 | 3539 |
| 8527 | 8538 | HTLV-1 | 3514 | 3602 | 3505 | 3602 |
| 1546 | 1564 | HTLV-2 | 3573 | 3626 | 3514 | 3626 |
| 2639 | 2652 | HTLV-2 | 3612 | 3633 | 3573 | 3633 |
| 5413 | 5429 | HTLV-2 | 3619 | 3678 | 3612 | 3678 |
| 1142 | 1155 | HTLV-2 | 3648 | 3679 | 3619 | 3679 |
| 2513 | 2550 | HTLV-2 | 3649 | 3682 | 3648 | 3682 |
| 7928 | 7940 | HTLV-2 | 3649 | 3682 | 3649 | 3682 |
| 5395 | 5407 | HTLV-2 | 3663 | 3685 | 3649 | 3685 |
| 3891 | 3905 | HEPATITIS C | 3666 | 3685 | 3663 | 3685 |
| 5506 | 5519 | HEPATITIS C | 3673 | 3749 | 3666 | 3749 |
| 7261 | 7273 | HEPATITIS C | 3724 | 3788 | 3673 | 3788 |
| 3804 | 3815 | HEPATITIS B | 3762 | 3795 | 3724 | 3795 |
| 7589 | 7600 | HEPATITIS B | 3765 | 3797 | 3762 | 3797 |
| 617 | 628 | HEPATITIS B | 3784 | 3815 | 3765 | 3815 |
| 4216 | 4261 | INFLUENZA A | 3804 | 3892 | 3784 | 3892 |
| 3648 | 3682 | SAIMIRI | 3880 | 3905 | 3804 | 3905 |
| 7198 | 7223 | ENTEROVIRUS | 3891 | 3938 | 3880 | 3938 |

TABLE 8F

| | | | | | |
|---|---|---|---|---|---|
| | Draft | Logical | | | |
| Respiratory Synctial Virus (RSV) | Rearrangement | Approach | Shared HIV Regions | Unique HIV Regions | # nucleotides |

| | | | | | | |
|---|---|---|---|---|---|---|
| 4703 | 4732 | RSV | 3910 | 3953 | 3891 | 3953 |
| 7214 | 7231 | RSV | 3941 | 3994 | 3910 | 3994 |
| 1493 | 1515 | RSV | 3968 | 4016 | 3941 | 4016 |
| 3101 | 3115 | RSV | 3971 | 4027 | 3968 | 4027 |
| 6314 | 6337 | RSV | 3995 | 4085 | 3971 | 4085 |
| 8377 | 8393 | RSV | 4060 | 4086 | 3995 | 4086 |
| 2171 | 2186 | RSV | 4068 | 4095 | 4060 | 4095 |
| 5955 | 5967 | RSV | 4075 | 4098 | 4068 | 4098 |
| 3666 | 3678 | RSV | 4079 | 4221 | 4075 | 4221 |
| 4645 | 4657 | RSV | 4174 | 4257 | 4079 | 4257 |
| 2285 | 2300 | RSV | 4192 | 4258 | 4174 | 4258 |
| 7754 | 7766 | RSV | 4193 | 4261 | 4192 | 4261 |
| 5559 | 5571 | RSV | 4206 | 4261 | 4193 | 4261 |
| 3006 | 6021 | RSV | 4216 | 4270 | 4206 | 4270 |
| 1256 | 1270 | YELLOW FEVER | 4225 | 4270 | 4216 | 4270 |
| 2192 | 2220 | YELLOW FEVER | 4234 | 4274 | 4225 | 4274 |
| 4485 | 4502 | YELLOW FEVER | 4236 | 4277 | 4234 | 4277 |
| 647 | 662 | WEST NILE VIRUS | 4246 | 4336 | 4236 | 4336 |
| 1423 | 1438 | WEST NILE VIRUS | 4251 | 4339 | 4246 | 4339 |
| 5941 | 5956 | WEST NILE VIRUS | 4289 | 4339 | 4251 | 4339 |
| 4641 | 4653 | WEST NILE VIRUS | 4325 | 4478 | 4289 | 4478 |
| 7486 | 7501 | WEST NILE VIRUS | 4466 | 4489 | 4325 | 4489 |
| 6774 | 6786 | WEST NILE VIRUS | 4478 | 4502 | 4466 | 4502 |
| 6153 | 6165 | WEST NILE VIRUS | 4485 | 4542 | 4478 | 4542 |
| 5050 | 5062 | VENEZUELAN EQUINE ENCEPHALITIS | 4517 | 4550 | 4485 | 4550 |
| 7056 | 7068 | VENEZUELAN EQUINE ENCEPHALITIS | 4517 | 4551 | 4517 | 4551 |
| 5826 | 5843 | VENEZUELAN EQUINE ENCEPHALITIS | 4526 | 4571 | 4517 | 4571 |
| 3880 | 3892 | VENEZUELAN EQUINE ENCEPHALITIS | 4535 | 4579 | 4526 | 4579 |

TABLE 8G

| | | | | | |
|---|---|---|---|---|---|
| | Draft | Logical | | | |
| SARS CORONNAVIRUS | Rearrangement | Approach | Shared HIV Regions | Unique HIV Regions | # nucleotides |

| | | | | | | |
|---|---|---|---|---|---|---|
| 6329 | 6343 | | 4563 | 4624 | 4535 | 4624 |
| 5321 | 5342 | | 4605 | 4651 | 4563 | 4651 |
| 6247 | 6263 | | 4637 | 4653 | 4605 | 4653 |
| 1777 | 1790 | | 4639 | 4657 | 4637 | 4657 |
| 1610 | 1623 | | 4641 | 4664 | 4639 | 4664 |
| 4563 | 4579 | | 4645 | 4665 | 4641 | 4665 |
| 8745 | 8758 | | 4653 | 4690 | 4645 | 4690 |
| 5720 | 5736 | | 4675 | 4700 | 4653 | 4700 |
| 6124 | 6137 | | 4688 | 4723 | 4675 | 4723 |
| 2456 | 2468 | | 4703 | 4732 | 4688 | 4732 |
| 575 | 587 | | 4712 | 4750 | 4703 | 4750 |
| 3358 | 3370 | | 4724 | 4751 | 4712 | 4751 |
| 5941 | 5953 | | 4738 | 4838 | 4724 | 4838 |
| 7717 | 7729 | | 4800 | 4928 | 4738 | 4928 |
| 2186 | 2203 | | 4895 | 4943 | 4800 | 4943 |
| 7454 | 7466 | | 4925 | 4951 | 4895 | 4951 |
| 4653 | 4665 | | 4932 | 5059 | 4925 | 5059 |
| 3132 | 3144 | | 5032 | 5059 | 4932 | 5059 |
| 6252 | 6267 | | 5040 | 5059 | 5032 | 5059 |
| 3091 | 3103 | | 5042 | 5062 | 5040 | 5062 |
| 1772 | 1784 | | 5042 | 5066 | 5042 | 5066 |
| 1277 | 1289 | | 5050 | 5137 | 5042 | 5137 |
| 2643 | 2655 | | 5112 | 5175 | 5050 | 5175 |
| 7582 | 7594 | RUBELLA | 5164 | 5208 | 5112 | 5208 |
| 3619 | 3633 | RABIES | 5196 | 5311 | 5164 | 5311 |
| 2715 | 2727 | RABIES | 5292 | 5320 | 5196 | 5320 |

TABLE 8H

| | | | | | |
|---|---|---|---|---|---|
| | Draft | Logical | | | |
| RHINOVIRUS | Rearrangement | Approach | Shared HIV Regions | Unique HIV Regions | # nucleotides |

| | | | | | | |
|---|---|---|---|---|---|---|
| 2312 | 2338 | RHINOVIRUS | 5308 | 5342 | 5292 | 5342 |
| 8449 | 8462 | RHINOVIRUS | 5321 | 5407 | 5308 | 5407 |
| 6059 | 6077 | RHINOVIRUS | 5395 | 5429 | 5321 | 5429 |
| 45 | 62 | RHINOVIRUS | 5413 | 5519 | 5395 | 5519 |
| 9130 | 9147 | RHINOVIRUS | 5506 | 5525 | 5413 | 5525 |
| 4738 | 4750 | RHINOVIRUS | 5514 | 5571 | 5506 | 5571 |
| 5196 | 5208 | RHINOVIRUS | 5550 | 5573 | 5514 | 5573 |
| 2816 | 2827 | RHINOVIRUS | 5559 | 5715 | 5550 | 5715 |
| 8452 | 8463 | RHINOVIRUS | 5606 | 5726 | 5559 | 5726 |
| 3784 | 3795 | RHINOVIRUS | 5669 | 5736 | 5606 | 5736 |
| 930 | 941 | RHINOVIRUS | 5689 | 5748 | 5669 | 5748 |
| 8352 | 8363 | RHINOVIRUS | 5689 | 5774 | 5689 | 5774 |
| 2975 | 2986 | RHINOVIRUS | 5701 | 5779 | 5689 | 5779 |
| 365 | 376 | RHINOVIRUS | 5720 | 5785 | 5701 | 5785 |

TABLE 8J

| | | | | | |
|---|---|---|---|---|---|
| | Draft | Logical | | | |
| ADENOVIRUS | Rearrangement | Approach | Shared HIV Regions | Unique HIV Regions | # nucleotides |

| | | | | | | |
|---|---|---|---|---|---|---|
| 8525 | 8544 | ADENOVIRUS | 5745 | 5814 | 5720 | 5814 |
| 4068 | 4085 | ADENOVIRUS | 5745 | 5817 | 5745 | 5817 |
| 5895 | 5908 | ADENOVIRUS | 5766 | 5822 | 5745 | 5822 |
| 2931 | 2944 | ADENOVIRUS | 5773 | 5822 | 5766 | 5822 |
| 1756 | 1773 | ADENOVIRUS | 5781 | 5823 | 5773 | 5823 |
| 3663 | 3679 | ADENOVIRUS | 5797 | 5825 | 5781 | 5825 |

TABLE 8K

| | | | | | |
|---|---|---|---|---|---|
| | Draft | Logical | | | |
| HERPESVIRUS | Rearrangement | Approach | Shared HIV Regions | Unique HIV Regions | # nucleotides |

| | | | | | | |
|---|---|---|---|---|---|---|
| 5988 | 6013 | HERPESVIRUS 1 | 5797 | 5837 | 5797 | 5837 |
| 678 | 695 | HERPESVIRUS 2 | 5799 | 5840 | 5797 | 5840 |
| 8517 | 8541 | HERPESVIRUS 2 | 5803 | 5843 | 5799 | 5843 |
| 7169 | 7189 | HERPESVIRUS 3 | 5815 | 5846 | 5803 | 5846 |
| 7901 | 7915 | HERPESVIRUS 3 | 5824 | 5848 | 5815 | 5848 |
| 6795 | 6809 | HERPESVIRUS 3 | 5826 | 5848 | 5824 | 5848 |
| 5040 | 5059 | HERPESVIRUS 3 | 5834 | 5856 | 5826 | 5856 |
| 5550 | 5573 | HERPESVIRUS 4 | 5842 | 5897 | 5834 | 5897 |
| 7454 | 7474 | HERPESVIRUS 4 | 5886 | 5908 | 5842 | 5908 |
| 4206 | 4221 | HERPESVIRUS 4 | 5895 | 5929 | 5886 | 5929 |
| 5842 | 5856 | HERPESVIRUS 4 | 5902 | 5930 | 5895 | 5930 |
| 232 | 251 | HERPESVIRUS 4 | 5902 | 5953 | 5902 | 5953 |
| 8524 | 8541 | HERPESVIRUS 4 | 5941 | 5956 | 5902 | 5956 |
| 4605 | 4624 | HERPESVIRUS 4 | 5941 | 5967 | 5941 | 5967 |
| 5799 | 5814 | HERPESVIRUS 5 | 5955 | 5984 | 5941 | 5984 |
| 3765 | 3788 | HERPESVIRUS 5 | 5973 | 6013 | 5955 | 6013 |
| 5042 | 5059 | HERPESVIRUS 5 | 5988 | 6021 | 5973 | 6021 |
| 2308 | 2322 | HERPESVIRUS 5 | 6033 | 6063 | 6033 | 6063 |
| 5292 | 5311 | HERPESVIRUS 5 | 6033 | 6068 | 6033 | 6068 |
| 8133 | 8147 | HERPESVIRUS 5 | 6045 | 6077 | 6033 | 6077 |
| 5042 | 5059 | HERPESVIRUS 5 | 6059 | 6117 | 6045 | 6117 |
| 5803 | 5817 | HERPESVIRUS 5 | 6124 | 6135 | 6124 | 6135 |
| 2599 | 2615 | HERPESVIRUS 6A | 6153 | 6165 | 6153 | 6165 |
| 1119 | 1134 | HERPESVIRUS 6A | 6165 | 6182 | 6153 | 6182 |
| 4675 | 4690 | HERPESVIRUS 6A | 6169 | 6197 | 6165 | 6197 |
| 3135 | 3155 | HERPESVIRUS 6A | 6198 | 6233 | 6198 | 6233 |
| 3105 | 3122 | HERPESVIRUS 6A | 6223 | 6237 | 6198 | 6237 |
| 8785 | 8804 | HERPESVIRUS 6A | 6247 | 6263 | 6247 | 6263 |
| 2571 | 2598 | HERPESVIRUS 6A | 6252 | 6267 | 6247 | 6267 |
| 1642 | 1656 | HERPESVIRUS 6A | 6272 | 6314 | 6272 | 6314 |
| 930 | 950 | HERPESVIRUS 7 | 6282 | 6321 | 6272 | 6321 |
| 2746 | 2760 | HERPESVIRUS 7 | 6282 | 6335 | 6282 | 6335 |
| 7855 | 7872 | HERPESVIRUS 7 | 6293 | 6337 | 6282 | 6337 |
| 1668 | 1686 | HERPESVIRUS 7 | 6302 | 6343 | 6293 | 6343 |
| 2170 | 2184 | HERPESVIRUS 7 | 6314 | 6368 | 6302 | 6368 |
| 4526 | 4550 | HERPESVIRUS 7 | 6329 | 6368 | 6314 | 6368 |
| 6165 | 6182 | HERPESVIRUS 7 | 6515 | 6534 | 6515 | 6534 |
| 6515 | 6534 | HERPESVIRUS 7 | 6542 | 6556 | 6542 | 6556 |
| 6223 | 6237 | HERPESVIRUS 7 | 6659 | 6671 | 6659 | 6671 |
| 6829 | 6844 | HERPESVIRUS 8 | 6660 | 6689 | 6659 | 6689 |
| 683 | 701 | HERPESVIRUS 8 | 6663 | 6691 | 6660 | 6691 |
| 1464 | 1483 | HERPESVIRUS 8 | 6717 | 6729 | 6717 | 6729 |
| 3206 | 3223 | HERPESVIRUS 8 | 6722 | 6764 | 6717 | 6764 |
| 8659 | 8673 | HERPESVIRUS 8 | 6734 | 6766 | 6722 | 6766 |
| 3000 | 3014 | HERPESVIRUS 8 | 6748 | 6786 | 6734 | 6786 |
| 6895 | 6912 | HERPESVIRUS 8 | 6774 | 6791 | 6748 | 6791 |
| 2981 | 2995 | HERPESVIRUS 8 | 6795 | 6809 | 6795 | 6809 |
| 8334 | 8353 | HERPESVIRUS 8 | 6815 | 6844 | 6815 | 6844 |
| 2178 | 2197 | HERPESVIRUS 8 | 6828 | 6855 | 6815 | 6855 |

TABLE 8 L

| | | | | | |
|---|---|---|---|---|---|
| | Draft | Logical | | | |
| PAPILLOMAVIRUS | Rearrangement | Approach | Shared HIV Regions | Unique HIV Regions | # nucleotides |

| | | | | | | |
|---|---|---|---|---|---|---|
| 1945 | 1963 | | 6865 | 6876 | 6865 | 6876 |
| 6717 | 6729 | | 6895 | 6912 | 6895 | 6912 |
| 4079 | 4098 | | 6942 | 6963 | 6942 | 6963 |
| 9062 | 9076 | | 6957 | 6968 | 6942 | 6968 |
| 2861 | 2872 | | 7053 | 7068 | 7053 | 7068 |
| 1491 | 1502 | | 7056 | 7078 | 7053 | 7078 |
| 2228 | 2239 | | 7074 | 7095 | 7056 | 7095 |
| 5164 | 5175 | | 7084 | 7101 | 7074 | 7101 |
| 7084 | 7095 | | 7108 | 7121 | 7108 | 7121 |
| 6542 | 6556 | | 7169 | 7189 | 7169 | 7189 |
| 1326 | 1337 | | 7179 | 7190 | 7169 | 7190 |
| 4535 | 4551 | | 7197 | 7223 | 7197 | 7223 |
| 3145 | 3161 | | 7198 | 7229 | 7197 | 7229 |
| 6957 | 6968 | | 7201 | 7230 | 7198 | 7230 |
| 6942 | 6963 | | 7202 | 7231 | 7201 | 7231 |
| 1293 | 1304 | ADENO | 7202 | 7242 | 7202 | 7242 |
| 700 | 711 | BOCAVIRUS | 7210 | 7242 | 7202 | 7242 |
| 1180 | 1196 | BOCAVIRUS | 7214 | 7247 | 7210 | 7247 |

TABLE 8 M

| | | | | | |
|---|---|---|---|---|---|
| Human | Draft | Logical | | | |
| Erythrovirus | Rearrangement | Approach | Shared HIV Regions | Unique HIV Regions | # nucleotides |

| | | | | | |
|---|---|---|---|---|---|
| 1145 | 1158 | 7261 | 7301 | 7261 | 7301 |
| 2231 | 2243 | 7263 | 7303 | 7261 | 7303 |
| 736 | 761 | 7265 | 7306 | 7263 | 7306 |
| 2182 | 2197 | 7272 | 7310 | 7265 | 7310 |
| 2231 | 2243 | 7274 | 7313 | 7272 | 7313 |
| 4246 | 4257 | 7292 | 7330 | 7274 | 7330 |
| 2969 | 2980 | 7315 | 7350 | 7292 | 7350 |
| 4325 | 4336 | 7454 | 7466 | 7454 | 7466 |
| 4075 | 4086 | 7454 | 7474 | 7454 | 7474 |
| 414 | 425 | 7486 | 7501 | 7486 | 7501 |
| 3612 | 3626 | 7559 | 7571 | 7559 | 7571 |
| 5886 | 5897 | 7564 | 7578 | 7559 | 7578 |
| 6865 | 6876 | 7582 | 7594 | 7582 | 7594 |
| 4712 | 4723 | 7589 | 7600 | 7582 | 7600 |
| 8145 | 8156 | 7717 | 7729 | 7717 | 7729 |

TABLE 8 N

| | | | | | |
|---|---|---|---|---|---|
| | Draft | Logical | | | |
| PARVOVIRUS | Rearrangement | Approach | Shared HIV Regions | Unique HIV Regions | # nucleotides |

| | | | | | |
|---|---|---|---|---|---|
| 8528 | 8541 | 7721 | 7747 | 7721 | 7747 |
| 4251 | 4277 | 7733 | 7762 | 7721 | 7762 |
| 2777 | 2797 | 7754 | 7766 | 7733 | 7766 |
| 196 | 207 | 7814 | 7836 | 7814 | 7836 |
| 5514 | 5525 | 7855 | 7872 | 7855 | 7872 |
| 5973 | 5984 | 7901 | 7915 | 7901 | 7915 |
| 4932 | 4943 | 7928 | 7940 | 7928 | 7940 |
| 1873 | 1887 | 7928 | 7940 | 7928 | 7940 |
| 685 | 696 | 7937 | 7960 | 7928 | 7960 |
| 7179 | 7190 | 7937 | 7960 | 7937 | 7960 |
| 3070 | 3081 | 8110 | 8121 | 8110 | 8121 |
| 8110 | 8121 | 8133 | 8147 | 8133 | 8147 |
| 1114 | 1125 | 8145 | 8156 | 8133 | 8156 |

TABLE 8 P

| | | | | | |
|---|---|---|---|---|---|
| | Draft | Logical | | | |
| DENGUE | Rearrangement | Approach | Shared HIV Regions | Unique HIV Regions | # nucleotides |

| | | | | | | |
|---|---|---|---|---|---|---|
| 7053 | 7078 | DENGUE-2 | 8334 | 8353 | 8334 | 8353 |
| 7564 | 7578 | DENGUE-2 | 8352 | 8363 | 8334 | 8363 |
| 7721 | 7735 | DENGUE-2 | 8377 | 8393 | 8377 | 8393 |
| 2754 | 2776 | DENGUE-2 | 8389 | 8421 | 8377 | 8421 |
| 4925 | 4951 | DENGUE-2 | 8449 | 8462 | 8449 | 8462 |
| 5824 | 5840 | DENGUE-2 | 8452 | 8463 | 8449 | 8463 |
| 4639 | 4651 | DENGUE-2 | 8516 | 8538 | 8516 | 8538 |
| 2559 | 2571 | DENGUE-2 | 8516 | 8541 | 8516 | 8541 |
| 3673 | 3685 | DENGUE-2 | 8517 | 8541 | 8516 | 8541 |
| 5781 | 5822 | DENGUE-2 | 8524 | 8541 | 8517 | 8541 |
| 1557 | 1570 | DENGUE-1 | 8525 | 8543 | 8524 | 8543 |
| 5834 | 5846 | DENGUE-1 | 8527 | 8544 | 8525 | 8544 |
| 5308 | 5320 | DENGUE-1 | 8527 | 8564 | 8527 | 8564 |
| 4688 | 4700 | DENGUE-1 | 8528 | 8574 | 8527 | 8574 |
| 2609 | 2621 | DENGUE-1 | 8547 | 8574 | 8528 | 8574 |
| 8864 | 8879 | DENGUE-1 | 8659 | 8673 | 8659 | 8673 |
| 2756 | 2773 | DENGUE-1 | 8684 | 8715 | 8684 | 8715 |
| 3505 | 3526 | DENGUE-3 | 8745 | 8758 | 8745 | 8758 |
| 7108 | 7121 | DENGUE-3 | 8785 | 8804 | 8785 | 8804 |
| 388 | 420 | DENGUE-3 | 8864 | 8879 | 8864 | 8879 |
| 3277 | 3289 | DENGUE-3 | 9002 | 9040 | 9002 | 9040 |
| 4466 | 4478 | DENGUE-3 | 9057 | 9073 | 9057 | 9073 |
| 7559 | 7571 | DENGUE-3 | 9062 | 9076 | 9057 | 9076 |
| 3941 | 3953 | DENGUE-3 | 9130 | 9147 | 9130 | 9147 |

1. A rapid test kit for detection of a DNA, an RNA or a fragment of a DNA or an RNA, the kit comprising:
a membrane;
at least one genetic probe immobilized on a test portion of the membrane such that, when a fluid containing the DNA, RNA or the fragment of the DNA or the RNA is directly filtered through the membrane, the at least one genetic probe immobilizes the DNA, RNA or the fragment of the DNA or the RNA; and
a staining agent selected such that, if the DNA, RNA or the fragment of the DNA or the RNA is present at a detectable level, the staining agent is immobilized preferentially on the test portion of the membrane such that a contrast is observable between the test portion and a background portion of the membrane.

2. The rapid test kit of claim 1, further comprising:
a destaining buffer selected to remove at least a portion of any non-specific background staining unrelated to binding between the at least one genetic probe, the DNA, RNA or the fragment of the DNA or the RNA, and the staining agent, such that the contrast is observable, if the DNA, RNA or the fragment of the DNA or the RNA is present at a detectable level.

3. The rapid test kit of claim 1, wherein the membrane is selected such that the membrane has a measured flow rate of a phosphate buffered saline from about 0.04 to about 0.4 mL/min/cm², using a modified ASTM standard flow rate measurement

4. The rapid test kit of claim 3, wherein the membrane is selected such that the membrane has a measured flow rate in a range from about 0.04 mL/min/cm² to about 0.2 mL/min/cm².

5. The rapid test kit of claim 4, wherein the measured flow rate of the membrane is in a range of at least 0.1 mL/min/cm² 0.2 mL/min/cm².

6. The rapid test kit of claim 1, wherein the staining agent comprises an oligonucleotide-functionalized nanoparticle or nanotube having an oligonucliotide capable of hybridizing at room temperature with the DNA, RNA or the fragment of the DNA or the RNA to be detected by the rapid test kit.

7. The rapid test kit of claim 6, wherein the at least one genetic probe includes a complimentary oligonucleotide for hybridization with a specific region of the DNA, RNA or the fragment of the DNA or the RNA.

8. The rapid test kit of claim 7, wherein the complimentary oligonucleotide is conjugated with a chitosan or a chitosan derivative such that the complimentary oligonucleotide is immobilized on the membrane.

9. The rapid test kit of claim 8, wherein the oligonucleotide-functionalized nanoparticle or nanotube comprises a gold nanoparticle functionalized by a thiolated oligonucleotide complementary to a different portion of the DNA, the RNA or the fragment of the DNA or the RNA than the portion of the DNA, the RNA or the fragment of the DNA or the RNA hybridized by the complimentary oligonucleotide immobilized on the membrane.

10. The rapid test kit of claim 9, wherein the thiolated oligonucleotide is a primer selected to hybridize a viral RNA selected from the group consisting of an HIV virus, a Hepatitis B virus, a Hepatitis C virus, a SARS virus and combinations thereof.

11. The rapid test kit of claim 10, wherein the primer is selected to hybridize the viral RNA of the HIV virus.

12. The rapid test kit of claim 11, wherein the complementary oligonucleotide immobilized on the membrane is selected to hybridize a region of the viral RNA of the HIV within the LTR sequence of the viral RNA of the HIV virus.

13. The rapid test kit of claim 6, wherein the staining agent comprises carbon nanotubes functionalized by oligonucleotides complementary with a portion of the DNA, the RNA, or the fragment of the DNA or the RNA.

14. The rapid test kit of claim 13, wherein the specific complementary DNA or viral RNA is a viral RNA selected from the group of viral RNA's consisting of an HIV virus, a Hepatitis B virus, a Hepatitis C virus, a SARS virus and combinations thereof.

15. The rapid test kit of claim 1, wherein the at least one genetic probe includes a 33-nt oligonucleotide from HIV 89.6 proviral clone.

16. The rapid test kit of claim 1, wherein the at least one genetic probe is conjugated with a chitosan or a chitosan derivative.

17. A method of using the rapid test kit of claim 1, comprising: detecting a detectable level of viral load in a sample volume of fluid.

18. The method of claim 17, further comprising: conjugating the at least one genetic probe with a chitosan or a chitosan derivative to form a conjugate; and immobilizing the conjugate on a test region of the membrane.

19. The method of claim 17, further comprising: illuminating the membrane with ultraviolet light to increase the contrast between the test portion and a background portion of the membrane.

20. The method of claim 17, further comprising reporting the level or concentration of the viral load in the sample volume of fluid.

21. The method of claim 20, wherein the step of reporting includes comparing the contrast or intensity of at least a portion of the test portion of the membrane to a standard.

22. The method of claim 17, wherein the step of detecting includes depositing the staining agent on the membrane such that a genetic probe in the staining agent binds selectively to the DNA, RNA or the fragment of the DNA or the RNA immobilized by the at least one genetic probe on the test portion of the membrane.

23. A test kit for detection of a DNA, an RNA or a fragment of a DNA or an RNA and at least one of the antibodies associated with the presence of the DNA or the RNA in a subject, the kit comprising:
a cellulose filter paper having a detection surface and an opposite surface, the cellulose filter paper selected from cellulose filter papers having a measured flow rate of a phosphate buffered saline from about 0.04 to about 0.4 mL/min/cm², using a modified ASTM standard flow rate measurement;
at least one genetic probe immobilized on a first test portion of the cellulose filter paper such that, when a fluid containing the DNA, RNA or the fragment of the DNA or the RNA is directly filtered through the cellulose filter paper, the at least one genetic probe immobilizes the DNA, RNA or the fragment of the DNA or the RNA;
at least one antibody detecting probe immobilized on a second test portion of the cellulose filter paper such that, when a fluid containing the antibody is directly filtered through the cellulose filter paper, the at least one antibody detecting probe immobilizes the antibody on the filter paper; and
at least one staining agent comprising an oligonucleotide coupled with a nanotube or a particle is selected such that, if the DNA, RNA or the fragment of the DNA or the RNA is present at a detectable level, the at least one staining agent is immobilized preferentially on the first test portion of the cellulose filter paper such that a contrast is observable between the first test portion and a background portion of the cellulose filter paper and if the antibody is present at a detectable level, then the at least one staining agent is immobilized preferentially on the second test portion of the cellulose filter paper such that a contrast is observable between the second test portion and the background portion of the cellulose filter paper.

24. The test kit of claim 23, wherein the at least one genetic probe is selected to distinguish the presence of the DNA, RNA or the fragment of the DNA or the RNA from the antibodies produced by administering a vaccine.

25. A test kit for detection of an antibody, the kit comprising:
a cellulose filter paper having a detection surface and an opposite surface, the cellulose filter paper selected from cellulose filter papers having a measured flow rate of a phosphate buffered saline from about 0.04 to about 0.4 mL/min/cm², using a modified ASTM standard flow rate measurement;
at least one antibody detecting probe immobilized on a test portion of the cellulose filter paper such that, when a fluid containing the antibody is directly filtered through the cellulose filter paper, the at least one antibody detecting probe immobilizes the antibody on the filter paper; and
at least one staining agent selected such that, if the antibody is present at a detectable level, the at least one staining agent is immobilized preferentially on the test portion of the cellulose filter paper such that a contrast is observable between the test portion and the background portion of the cellulose filter paper.

26. The test kit of claim 25, wherein the at least one antibody detecting probe or the at least one staining agent includes a gp41 peptide fragment comprising SEQ. ID. NO. 14.

27. A test for a disease, comprising:
at least one genetic probe deposited on a detection region of a glass slide such that, when a fluid containing DNA, RNA or a fragment of the DNA or the RNA is directly deposited on the detection region of the glass slide, the DNA, RNA or a fragment of the DNA or the RNA is hybridized by the at least one genetic probe;
a suspension of nanotubes or particles functionalized by a complementary oligonucleotide such that, when the suspension is directly deposited on the detection region of the glass slide, the complimentary oligonucleotide hybridizes the DNA, RNA or the fragment of the DNA or the RNA; and
a detector for detecting the emission of a light from the detection region or the absorbtion of a light by the detection region of the glass slide.

28. The test of claim 27, wherein the suspension includes carbon nanotubes functionalized by the complementary oligonucleotide.

29. The test of claim 28, wherein the detector includes an ultraviolet light source; and the detector detects the level of emissions of fluorescent or phosphorescent light emitted from the detection region such that the viral load of DNA, RNA or the fragment of the DNA or the RNA in a tested sample is capable of being determined quantitatively.

30. The test of claim 29, wherein the detector measures the fluorescence from the detection region.

31. The test of claim 30, wherein the detector reports an output associated with a viral load.

32. The test of claim 27, wherein the suspension includes gold nanoparticles.

33. The test of claim 32, wherein the detector measures absorbtion of a light through the detection region.

34. The test of claim 27, wherein the genetic probe includes a 33-nt oligonucleotide from HIV 89.6 proviral clone.

35. A test kit for detection of an antibody, the kit comprising:
a cellulose filter paper having a detection surface and an opposite surface, the cellulose filter paper selected from cellulose filter papers having a measured flow rate of a phosphate buffered saline from about 0.04 to about 0.4 mL/min/cm², using a modified ASTM standard flow rate measurement;
at least one antibody detecting probe immobilized on a test portion of the cellulose filter paper such that, when a fluid containing the antibody is directly filtered through the cellulose filter paper, the at least one antibody detecting probe immobilizes the antibody on the filter paper; and
at least one staining agent selected such that, if the antibody is present at a detectable level, the at least one staining agent is immobilized preferentially on the test portion of the cellulose filter paper such that a contrast is observable between the test portion and the background portion of the cellulose filter paper.

36. The test kit of claim 35, wherein the at least one antibody detecting probe or the at least one staining agent includes a gp41 peptide fragment.

37. The test kit of claim 36, wherein the gp41 peptide fragment consists of SEQ. ID. NO. 14.

38. The test kit of claim 37, wherein the at least one staining buffer includes a Protein A coupled to colloidal gold.

39. The test kit of claim 35, further comprising:
a genetic probe immobilized on another portion of the cellulose filter paper; and
the at least one staining agent includes a complementary-oligonucleotide-functionalized nanotube or oligonucleotide functionalized particle such that, when a fluid containing DNA, RNA or a fragment of the DNA or the RNA is directly filtered through the cellulose filter paper, the at least one genetic probe immobilizes the DNA, RNA or the fragment of the DNA or the RNA, and the staining agent hybridizes with the DNA, RNA or the fragment of the DNA or the RNA, if the genetic probe and the staining agent include an oligonucleotide complementary to the DNA, RNA or the fragment of the DNA or the RNA, providing contrast between the another portion of the cellulose filter paper and a background portion of the cellulose filter paper.

40. The test kit of claim 39, wherein the genetic probe is complimentary oligonucleotide hybridizing the DNA, RNA or the fragment of the DNA or the RNA.

41. The test kit of claim 40, wherein the complimentary oligonucleotide hybridizes a portion of the LTR sequence of the HIV-1 virus.

42. The test kit of claim 41, wherein the at least one staining agent comprises a plurality of thiolated oligonucleotides coupled with gold nanoparticles selected such that the plurality of thiolated oligonucleotides hybridize a plurality of portions of the RNA of the HIV-1 virus.

43. A method comprising:
administrating a vaccine; and
using the test kit of claims 1, 23, 25, 35, the test of claim 27, or a combination thereof, wherein the step of using includes evaluating the vaccine safety or effectiveness.
